(19) United States
(12) Patent Application Publication
(10) Pub. No.: US 2023/0383288 A1
(43) Pub. Date: Nov. 30, 2023

(54) SYSTEMS, METHODS, AND COMPOSITIONS FOR RNA-GUIDED RNA-TARGETING CRISPR EFFECTORS

(71) Applicants: Massachusetts Institute of Technology, Cambridge, MA (US); The University of Tokyo, Tokyo (JP)

(72) Inventors: Omar Osama Abudayyeh, Cambridge, MA (US); Jonathan S. Gootenberg, Cambridge, MA (US); Wenyuan Zhou, Cambridge, MA (US); Hiroshi Nishimasu, Tokyo (JP); Kazuki Kato, Tokyo (JP); Cian Franz Schmitt-Ulms, Cambridge, MA (US)

(21) Appl. No.: 18/322,675

(22) Filed: May 24, 2023

Related U.S. Application Data
(60) Provisional application No. 63/365,281, filed on May 25, 2022.

(51) Int. Cl. C12N 15/11; C12N 9/22; C12N 15/86

(52) U.S. Cl. CPC C12N 15/11; C12N 9/22; C12N 15/86; C12N 2310/20

(57) ABSTRACT

This disclosure provides systems, methods, and compositions for RNA-guided RNA-targeting CRISPR effectors for the treatment of diseases, and for use as diagnostics. In addition, nucleotide deaminase functionalized CRISPR systems for RNA editing RNA knockdown, viral resistance, splicing modulation, RNA tracking, translation modulation, and epi-transcriptomic modifications are disclosed.

### CROSS REFERENCE TO RELATED APPLICATION

This application claims the benefit of U.S. Provisional Patent Application Ser. No. 63/365,281, filed May 25, 2022. The entire contents of the above-referenced patent application is incorporated by reference in its entirety herein.

### STATEMENT REGARDING FEDERALLY SPONSORED RESEARCH OR DEVELOPMENT

This invention was made with government support under AI149694 and HG011857 awarded by National Institutes of Health. The government has certain rights in the invention.

### SEQUENCE LISTING

The instant application contains a Sequence Listing which has been submitted electronically in .xml format and is hereby incorporated by reference in its entirety. The .xml copy, created on May 18, 2023, is named 739749083474-034 and is 341,432 bytes in size.

### TECHNOLOGY FIELD

The subject matter disclosed herein is generally related to systems, methods, and compositions for RNA-guided RNA-targeting CRISPR effectors for the treatment of diseases and diagnostics.

### BACKGROUND

RNA-targeting tools are important for studying RNA biology, for engineering genes, and for developing RNA therapeutics, among others. These tools can regulate intracellular and intercellular target-gene function and expression as well as manipulate specific target-genomic information. Few RNA-targeting tools have been developed, and those that have can present challenges. For instance, some have weak activity in mammalian cells, and some have collateral effects, which can be toxic in certain cell types. In some circumstances, the size of the RNA-targeting tools can be a barrier to their use. Delivery of the RNA-targeting tools can also be difficult. Thus, there remains a need for effective tools for RNA-targeting tools and their delivery.

### SUMMARY

The present disclosure provides compositions and systems for RNA-guided RNA-targeting CRISPR effectors for the treatment of diseases and diagnostics.

In one aspect, provided herein is a polypeptide comprising an amino acid sequence at least 85% identical to the amino acid sequence of any one of SEQ ID NOs: 1-4. The amino acid sequence of the polypeptide comprises at least one amino acid modification or mutation relative to the amino acid sequence of SEQ ID NO: 1-4.

In some embodiments, the amino acid sequence of the polypeptide is at least 85%, at least 90%, at least 95%, or at least 99% identical to the amino acid sequence of SEQ ID NO: 1-4.

In some embodiments, the amino acid sequence of the polypeptide comprises the amino acid sequence of SEQ ID NO: 1-4.

In some embodiments, the at least one amino acid modification or mutation comprises: removing an amino acid; adding an amino acid; replacing an amino acid with no charge with an amino acid with a positive charge; or replacing an amino acid with a negative charge with an amino acid with a positive charge.

In some embodiments, the amino acid without charge is selected from the group consisting of serine, threonine, asparagine, glutamine, cysteine, glycine, proline, alanine, valine, isoleucine, leucine, methionine, phenylalanine, tyrosine, and tryptophan.

In some embodiments, the amino acid with a negative charge is selected from the group consisting of aspartic acid and glutamic acid.

In some embodiments, the amino acid with a positive charge is selected from the group consisting of arginine, histidine, and lysine.

In some embodiments, the amino acid sequence of the polypeptide comprises 1, 2, 3, or 4 amino acid modifications or mutations.

In some embodiments, the amino acid sequence of the polypeptide comprises an alanine at a position corresponding to position 43 of SEQ ID NO: 1; an alanine at a position corresponding to position 55 of SEQ ID NO: 55; and/or an alanine at a position corresponding to position 152 of SEQ ID NO: 1.

In some embodiments, the polypeptide comprises a deletion of one or more amino acid residues at positions 979 through 1293 of SEQ ID NO: 1; at positions 1007 through 1220 of SEQ ID NO: 1; and/or at positions 1146 through 1211 of SEQ ID NO: 1.

In another aspect, provided herein is a composition that cleaves an RNA target comprising a guide RNA that specifically hybridizes to the RNA target and a polypeptide.

In some embodiments, the guide RNA comprises a mismatch distance that is about 20-65% of the length of the guide.

In some embodiments, the guide RNA has a sequence with a length of from about 20 to about 53 nucleotides (nt), optionally from about 25 to about 53 nt, more optionally from about 29 to about 53 nt, or optionally from about 40 to about 50 nt.

In some embodiments, the guide RNA is a pre-crRNA.

In some embodiments, the guide RNA is a mature crRNA.

In some embodiments, the RNA target is a single-strand RNA (ssRNA).

In some embodiments, the RNA target is in a cell.

In some embodiments, the cell is a prokaryotic cell.

In some embodiments, the cell is a eukaryotic cell.

In some embodiments, the eukaryotic cell is a mammalian cell.

In some embodiments, the mammalian cell is a human cell.

In some embodiments, the guide RNA comprises a mismatch that is about 20 to about 30 nucleotides from a non-pairing C of the guide RNA.

In another aspect, provided herein is a nucleic acid molecule encoding a polypeptide.

In some embodiments, the nucleic acid molecule encodes the guide RNA.

In some embodiments, the nucleic acid molecule further comprises a nucleic acid molecule that encodes the guide RNA.

In another aspect, provided herein is a vector comprising the nucleic acid molecule.

In some embodiments, the vector is a viral vector.

In some embodiments, the viral vector is a lenti-associated viral vector, baculo-associated viral vector, or adeno-associated viral vector.

In some embodiments, the viral vector is derived from a virus selected from the group consisting of Myoviridae, Siphoviridae, Podoviridae, Corticoviridae, Lipothrixviridae, Poxviridae, Iridoviridae, Adenoviridae, Polyomaviridae, Papillomaviridae, Mimiviridae, Pandoravirusa, Salterprovirusa, Inoviridae, Microviridae, Parvoviridae, Circoviridae, Hepadnaviridae, Caulimoviridae, Retroviridae, Cystoviridae, Reoviridae, Bimaviridae, Totiviridae, Partitiviridae, Filoviridae, Orthomyxoviridae, Deltavirusa, Leviviridae, Picomaviridae, Mamaviridae, Secoviridae, Potyviridae, Caliciviridae, Hepeviridae, Astroviridae, Nodaviridae, Tetraviridae, Luteoviridae, Tombusviridae, Coronaviridae, Arteriviridae, Flaviviridae, Togaviridae, Virgaviridae, Bromoviridae, Tymoviridae, Alphaflexiviridae, Sobemovirusa, Idaeovirusa, and Herpesviridae.

In another aspect, provided herein is a cell comprising a polypeptide, a composition, a nucleic acid molecule, and/or a vector.

In some embodiments, the cell is a prokaryotic cell.

In some embodiments, the cell is a eukaryotic cell.

In some embodiments, the eukaryotic cell is a mammalian cell.

In some embodiments, the mammalian cell is a human cell.

In another aspect, provided herein is a method of cleaving an RNA target in a cell comprising providing to the cell a polypeptide, a composition, a nucleic acid molecule, and/or a vector.

In another aspect, provided herein is a method of stabilizing an RNA target in a cell comprising providing to the cell a polypeptide, a composition, a nucleic acid molecule, and/or the vector.

In another aspect, provided herein is a method of affecting translation of an RNA target in a cell comprising providing to a polypeptide, a composition, a nucleic acid molecule, and/or a vector.

In some embodiments, the RNA target is an ssRNA.

In another aspect, provided herein is a method of treating a genetically inherited disease in a subject in need thereof comprising administering to the subject an effective amount of a polypeptide, a composition, a nucleic acid molecule, and/or a vector, wherein the genetically inherited disease involves a guanosine to adenosine change in a genome of the subject.

In some embodiments, the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; cancer; Uveitis; SCA1; SCA2; FUS-Amyotrophic Lateral Sclerosis (ALS); MAPT-Frontotemporal Dementia (FTD); Myotonic Dystrophy Type 1 (DM1); Diabetic Retinopathy (DR/DME); Oculopharyngeal Muscular Dystrophy (OPMD); SCA8; C9ORF72-Amyotrophic Lateral Sclerosis (ALS); SOD1-Amyotrophic Lateral Sclerosis (ALS); Spinal Cord Injury (targets: mTOR, PTEN, KLF6/7, SOX11, KCC2, and growth factors); SCA6; SCA3 (Machado-Joseph Disease); Multiple system Atrophy (MSA); Treatment-resistant Hypertension; Myotonic Dystrophy Type 2 (DM2); Fragile X-associated Tremor Ataxia Syndrome (FXTAS); West Syndrome with ARX Mutation; Age-related Macular Degeneration (AMD)/Geographic Atrophy (GA); C9ORF72-Frontotemporal Dementia (FTD); Facioscapulohuneral Muscular Dystrophy (FSHD); Fragile X Syndrome (FXS); Huntington's Disease; Glaucoma; Acromegaly; Achromatopsia (total color blindness); Ullrich congenital muscular dystrophy; Hereditary myopathy with lactic acidosis; X-linked spondyloepiphyseal dysplasia tarda; Neuropathic pain (Target: CPEB); Persistent Inflammation and injury pain (Target: PABP); Neuropathic pain (Target: miR-30c-5p); Neuropathic pain (Target: miR-195); Friedreich's Ataxia; Uncontrolled gout; Inflammatory pain (Target: Nav1.7 and Nav1.8); Choroideremia; Focal epilepsy; Alpha-1 Antitrypsin deficiency (AATD); Androgen Insensitivity Syndrome; Opioid-induced hyperalgesia (Target: Raf-1); Neurofibromatosis type 1; Stargardt's Disease; Dravet Syndrome; Retinitis Pigmentosa; and Parkinson's Disease.

In another aspect, provided herein is a method of treating a genetically inherited disease in a subject in need thereof comprising administering to the subject an effective amount of a polypeptide, a composition, a nucleic acid molecule, and/or a vector, wherein the genetically inherited disease is a pre-termination disease.

In another aspect, provided herein is a method of altering splicing of a pre-mRNA in a cell comprising administering to the cell an effective amount of a polypeptide, a composition, a nucleic acid molecule, and/or a vector.

In another aspect, provided herein is a method of changing microRNA targets in a subject in need thereof comprising administering to the subject an effective amount of a polypeptide, a composition, a nucleic acid molecule, and/or a vector.

In another aspect, provided herein is a method of increasing RNA stability in a cell comprising administering to the cell an effective amount of a polypeptide, a composition, a nucleic acid molecule, and/or a vector.

In another aspect, provided herein is a method of modulating translation in a cell comprising administering to the cell an effective amount of a polypeptide, a composition of, a nucleic acid molecule, and/or a vector.

In another aspect, provided herein is a method of detecting a bacterium or derivative thereof in a sample, the method comprising: adding to the sample an effective amount of a polypeptide, a composition, a nucleic acid molecule, and/or a vector; and detecting a reporter specific to the bacterium or derivative thereof.

In another aspect, provided herein is a method of detecting a virus or derivative thereof in a sample, the method comprising: adding to the sample an effective amount of a polypeptide, a composition, a nucleic acid molecule, and/or a vector; and detecting a reporter specific to the virus or derivative thereof.

These and other aspects of the applicants' teaching are set forth herein.

### BRIEF DESCRIPTION OF THE DRAWINGS

Aspects, features, benefits and advantages of the embodiments described herein will be apparent with regard to the following description, appended claims, and accompanying drawings where:

FIG. 1A is a schematic diagram of a domain structure of Cas7-11;

FIG. 1B is a schematic diagram of nucleotide sequences of a crRNA (SEQ ID NO: 8) and its target RNA (SEQ ID NO: 5), wherein a pre-crRNA processing site and target RNA cleavage sites are indicated by cyan, and yellow and green triangles, respectively, and a crRNA and target RNA contain a 5′ GG for in vitro transcription;

FIG. 1C a ribbon representation of an overall structure of a Cas7-11-crRNA-target RNA complex, wherein zinc ions bound to the Cas7.1-Cas7.4 domains are shown as orange spheres, the disordered regions are indicated as dotted lines, and the disordered L1 and L2 linkers are not shown for clarity;

FIG. 1D is a surface representation of a Cas7-11-crRNA-target RNA complex;

FIG. 2A is a ribbon representation of a Cas7.1 domain;

FIG. 2B is a ribbon representation of a Cas7.2 domain;

FIG. 2C is a ribbon representation of a Cas7.3 domain;

FIG. 2D is a ribbon representation of a Cas7.4 domain;

FIG. 2E is a ribbon representation of a Cas11 domain;

FIG. 2F is a ribbon representation of an INS domain;

FIG. 3A is a schematic representation of crRNA and target RNA structures;

FIG. 3B is a schematic representation of interactions between Cas7-11 and bound nucleic acids, wherein Cas7-11 residues that interact with nucleic acids through their main chains are shown in parentheses, and a pre-crRNA processing site and target RNA cleavage sites are indicated by cyan, and yellow and green triangles, respectively;

FIG. 4A is a surface representation of a crRNA 5′ tag region, wherein a pre-crRNA processing site is indicated by a cyan triangle, and pGp is guanosine-3′,5′-diphosphate;

FIG. 4B is a ribbon representation of a C(−1)-G(−4) region in a crRNA 5′ tag;

FIG. 4C is a ribbon representation of a C(−6) region in a crRNA 5′ tag;

FIG. 4D is a ribbon representation of a A(−7)-U(−9) region in a crRNA 5′ tag;

FIG. 4E is a ribbon representation of a G(−10)-U(−14) region in a crRNA 5′ tag;

FIG. 4F is a ribbon representation of a pGp molecule, wherein densities for the pGp and crRNA molecules are shown as gray meshes, and a pre-crRNA processing site is indicated by a cyan triangle;

FIG. 4G is a schematic representation and an image of an electrophoresis gel for an in vitro pre-crRNA processing by WT Cas7-11 and Cas7-11 mutants, wherein target RNA cleavage activities were measured using a mature crRNA containing a 14-nt 5′ tag with a 5′ GG, or a pre-crRNA containing a 23-nt 5′ tag with a 5′ GG TBC;

FIG. 4H is a schematic representation and an image of an electrophoresis gel for an in vitro target RNA cleavage by WT Cas7-11 and Cas7-11 mutants;

FIG. 5A is a surface representation of a guide-target duplex by Cas7.4/INS/CTE domains, wherein target RNA cleavage sites (sites 1 and 2) are indicated by yellow and green triangles;

FIG. 5B is a ribbon representation of a guide-target duplex by Cas7.4/INS/CTE domains, wherein target RNA cleavage sites (sites 1 and 2) are indicated by yellow and green triangles;

FIG. 5C is a surface representation of a guide-target duplex by Cas7.2-Cas7.4/Cas11 domains, wherein target RNA cleavage sites (sites 1 and 2) are indicated by yellow and green triangles;

FIG. 5D is a ribbon representation of a guide-target duplex by Cas7.2-Cas7.4/Cas11 domains, wherein target RNA cleavage sites (sites 1 and 2) are indicated by yellow and green triangles;

FIG. 5E is a schematic representation and an image of an electrophoresis gel for an in vitro target RNA cleavage by a WT Cas7-11 using mismatch-containing crRNAs;

FIG. 6A are ribbon representations of Cas7-11 variants;

FIG. 6B are images of an electrophoresis gel for an in vitro target RNA cleavage by truncated Cas7-11 variants;

FIG. 6C is a diagram showing the knockdown of Gluc mRNA by truncated Cas7-11 variants in HEK293FT cells, wherein data are normalized to non-targeting controls and shown as mean±s.e.m. (n=3);

FIG. 6D is a diagram showing the knockdown of endogenous mRNA transcripts by truncated Cas7-11 variants in HEK293FT cells, wherein data are normalized to non-targeting controls and shown as mean±s.e.m. (n=3);

FIG. 6E are schematic representations of an AAV design;

FIG. 6F is a schematic representation an AAV experimental;

FIG. 6G is a diagram showing the knockdown of Gluc mRNA by truncated Cas7-11 variants in HEK293FT cells via AAV delivery, wherein data are normalized to non-targeting controls and shown as mean±s.e.m. (n=3);

FIG. 7A are schematic representations of a structure of a type III-E Cas7-11 complex, wherein guide-target duplexes are shown on the left of the complexes;

FIG. 7B are schematic representations of a structure of a type III-A Csm complex, wherein guide-target duplexes are shown on the left of the complexes;

FIG. 8A is a schematic representation of a single-particle cryo-EM image processing workflow;

FIG. 8B is a graph showing a Fourier shell correlation curve calculated between half-maps in a 3D reconstruction;

FIG. 8C is a graph showing a Fourier shell correlation curve calculated between a refined model and a density map;

FIG. 8D is a surface representation of a local resolution of a density map;

FIG. 9A are ribbon representations of Cas7.2/Cas7.3 domains of Cas7-11 and Csm3 subunits of Csm complex (PDB ID: 6IFY);

FIG. 9B are ribbon representations of Cas11 domain of Cas7-11 and Csm2 subunit of Csm complex (PDB ID: 6IFY);

FIG. 9C are ribbon representations of INS domain of Cas7-11 and Bacillus subtilis cold shock protein (CSP) (PDB ID: 1CSP), wherein the INS domain contains the two five-stranded β-barrels similar to cold shock proteins;

FIG. 10A (SEQ ID NO:110, 111, 112, 113) shows multiple sequence alignments of Cas7-11 orthologs wherein the alignments were prepared using Clustal Omega (http://www.ebi.ac.uk/Tools/msa/clustalo) and ESPript3 (http://espript.ibcp.fr/ESPript/ESPript), and key residues are marked triangles,

FIG. 10B (SEQ ID NO:110, 111, 112, 113) shows multiple sequence alignments of Cas7-11 orthologs wherein the alignments were prepared using Clustal Omega (http://www.ebi.ac.uk/Tools/msa/clustalo) and ESPript3 (http://espript.ibcp.fr/ESPript/ESPript), and key residues are marked triangles;

FIG. 10C (SEQ ID NO:110, 111, 112, 113) shows multiple sequence alignments of Cas7-11 orthologs wherein the alignments were prepared using Clustal Omega (http://www.ebi.ac.uk/Tools/msa/clustalo) and ESPript3 (http://espript.ibcp.fr/ESPript/ESPript), and key residues are marked triangles;

FIG. 11A is a surface representation of Cas7.1/Cas7.3 domain, wherein the crRNA and target RNA are omitted for clarity;

FIG. 11B is a surface representation of Cas7.2/Cas7.4/INS domain, wherein the crRNA and target RNA are omitted for clarity;

FIG. 11C is a surface representation of Cas11/CTE domain, wherein the crRNA and target RNA are omitted for clarity;

FIG. 11D is a surface representation of Cas11/L4 domain, wherein the crRNA and target RNA are omitted for clarity;

FIG. 11E is a schematic representation of Cas7-11 domains;

FIG. 12 is a density map (unsharpened, FSC-weighted) for bound RNA molecules (stereo view);

FIG. 13A is a ribbon representation of a guide-target duplex by the Cas7.2/Cas7.3/Cas11 domains of type III-E Cas7-11, wherein target RNA cleavage sites (sites 1 and 2) are indicated by yellow and green triangles, respectively;

FIG. 13B is a ribbon representation of a guide-target duplex by the Csm1/Csm2/Csm3 subunits of type III-A Csm (stereoview), wherein target RNA cleavage sites (sites 1 and 2) are indicated by yellow and green triangles, respectively;

FIG. 13C shows the superposition of the Cas7-11 and Csm complexes (stereo view);

FIG. 14 shows a schematic representation and an image of an electrophoresis gel for the pr-crRNA processing by DiCas7-11 mutants;

FIG. 15 shows a schematic representation and an image of an electrophoresis gel for the target ssRNA cleavage by pre-mature and mature crRNA with DiCas7-11 mutants;

FIG. 16 shows a schematic representation and images of electrophoresis gels for the target ssRNA cleavage by mismatched crRNA guides and DiCas7-11;

FIG. 17 shows images of electrophoresis gels for the target ssRNA cleavage by mismatched crRNA guides and DiCas7-11 mutants;

FIG. 18A shows images of electrophoresis gels for the target ssRNA cleavage by mismatched crRNA guides and mismatched target;

FIG. 18B shows images of electrophoresis gels for the target ssRNA cleavage by mismatched crRNA guides and mismatched target;

FIG. 19A is a schematic representation of the target ssRNA cleavage by DiCas7-11 processing mutants and truncated DiCas7-11;

FIG. 19B shows images of electrophoresis gels for the target ssRNA cleavage by DiCas7-11 processing mutants and truncated DiCas7-11;

FIG. 20A shows a schematic representation of the knockdown of Gluc mRNA in HEK293FT cells by truncated DiCas7-11;

FIG. 20B shows a diagram for the knockdown of Gluc mRNA in HEK293FT cells by truncated DiCas7-11;

FIG. 21A shows a diagram for the knockdown of endogenous mRNA in HEK293FT cells by truncated DiCas7-11 for PPIB transcript;

FIG. 21B shows a diagram for the knockdown of endogenous mRNA in HEK293FT cells by truncated DiCas7-11 for MALAT1 transcript;

FIG. 21C shows a diagram for the knockdown of endogenous mRNA in HEK293FT cells by truncated DiCas7-11 for transcript;

FIG. 22A shows A diagram for the knockdown of Gluc mRNA in HEK293FT cells by truncated DiCas7-11 packaged in AAV8 vector;

FIG. 22B shows a diagram for the knockdown of Gluc mRNA in HEK293FT cells by truncated DiCas7-11 packaged in AAV8 vector;

FIG. 23 shows a schematic representation and an intensity graph for the knockdown of Gluc mRNA in HEK293FT cells by DiCas7-11 and mismatched crRNA guides;

FIG. 24 shows a ribbon representation of DiCas7-11 and the location of residue mutations;

FIG. 25 shows a diagram of single mutant g-luciferase knockdown;

FIG. 26 shows a diagram of single mutant endogenous MALAT1 knockdown;

FIG. 27 shows a diagram of single mutant endogenous PPIB knockdown;

FIG. 28 shows a diagram of single and double mutant G-luciferase knockdown;

FIG. 29 shows a diagram of saturation mutagenesis for D1530 residue; and

FIG. 30 shows a diagram of single, double, triple, and quadruple mutants G-luciferase knockdown.

### DETAILED DESCRIPTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes I X, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an,” and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +1-5% or less, +/−1% or less, +1-0.5% or less, and +/−0.1% or less of and from the specified value, insofar such variations are appropriate to perform in the disclosed disclosure. It is to be understood that the value to which the modifier “about” or “approximately” refers is itself also specifically, and preferably, disclosed.

Overview

The embodiments disclosed herein provide (non-naturally occurring or engineered) constructs, compositions, systems, and methods for site-directed RNA editing of RNA molecules. For example, the present disclosure provides (non-naturally occurring or engineered) methods for inhibiting intra and inter-cellular signaling pathways by modification of post-translational modification sites on select target RNA molecules. In certain embodiments, the present disclosure provides (non-naturally occurring or engineered) methods for inhibiting intracellular phosphorylation of serine, threonine and tyrosine residues by editing the genetic codon of these amino acids by means of site-directed RNA editing or RNA molecules. Embodiments disclosed herein further provide methods of inhibiting pathological activation of cell signaling mediated by post-translational modifications, such as phosphorylation, which are involved in many diseases, including cancer, immunodeficiency, infectious diseases, inflammatory disorders and neurodegenerative disorders. The RNA-editing modification may be aimed at a single post-translational modification site of a single gene and can also be multiplexed by targeting multiple sites on the same or different genes to increase efficacy. These approaches may be further combined with other treatments such as radiation, chemotherapy, targeted therapy based on antibodies or small molecules, and immunotherapy, which may have a synergistic effect.

The embodiments disclosed herein provide (non-naturally occurring or engineered) systems, constructs, and methods for targeted base editing. In general, the systems disclosed herein comprise a targeting component and a base editing component. The targeting component may function to specifically target the base editing component to a target nucleotide sequence in which one or more nucleotides are to be edited. The base editing component may then catalyze a chemical reaction to convert a first nucleotide in the target sequence to a second nucleotide. For example, the base editor may catalyze conversion of an adenine such that it is read as guanine by a cell's transcription or translation machinery, or vice versa. Likewise, the base editing component may catalyze conversion of cytidine to an uracil, or vice versa. In certain example embodiments, the base editor may be derived by starting with a known base editor, such as an adenine deaminase or cytidine deaminase, and modified using methods such as directed evolution to derive new functionalities. Directed evolution techniques are known in the art and may include those described in WO 2015/184016 “High-Throughput Assembly of Genetic Permutations.”

Compositions and Systems

The present disclosure provides (non-naturally occurring or engineered) systems for editing a nucleic acid such as a gene or a product thereof (e.g., the encoded RNA or protein).

In some embodiments, the systems may be an engineered, non-naturally occurring system suitable for modifying post-translational modification sites on proteins encoded by a target nucleic acid sequence. In certain cases, the target nucleic acid sequence is RNA, e.g., mRNA or a fragment thereof. In certain cases, the target nucleic acid sequence is DNA, e.g., a gene or a fragment thereof. In general, the system may comprise one or more of a catalytic inactive (dead) Cas protein (e.g., dead Cas7-11), a nucleotide deaminase protein or catalytic domain thereof, and a guide molecule. In certain examples, the nucleotide deaminase protein may be an adenosine deaminase. In certain examples, the nucleotide deaminase protein may be a cytidine deaminase. The guide sequence may be designed to have a degree of complementarity with a target sequence at one or more codons comprising an adenine or cytidine and that is post-translationally modified.

CRISPR-Cas

Some embodiments disclosed herein are directed to CRISPR-Cas (clustered regularly interspaced short palindromic repeats associated proteins) systems. In the conflict between bacterial hosts and their associated viruses, CRISPR-Cas systems provide an adaptive defense mechanism that utilizes programmed immune memory. CRISPR-Cas systems provide their defense through three stages: adaptation, the integration of short nucleic acid sequences into the CRISPR array that serves as memory of past infections; expression, the transcription of the CRISPR array into a pre-crRNA (CRISPR RNA) transcript and processing of the pre-crRNA into functional crRNA species targeting foreign nucleic acids; and interference, the programming of CRISPR effectors by crRNA to cleave nucleic acid of foreign threats. Across all CRISPR-Cas systems, these fundamental stages display enormous variation, including the identity of the target nucleic acid (either RNA, DNA, or both) and the diverse domains and proteins involved in the effector ribonucleoprotein complex of the system.

CRISPR-Cas systems can be broadly split into two classes based on the architecture of the effector modules involved in pre-crRNA processing and interference. Class 1 systems have multi-subunit effector complexes composed of many proteins, whereas Class 2 systems rely on single-effector proteins with multi-domain capabilities for crRNA binding and interference; Class 2 effectors often provide pre-crRNA processing activity as well. Class 1 systems contain 3 types (type I, III, and IV) and 33 subtypes, including the RNA and DNA targeting type III-systems. Class 2 CRISPR families encompass 3 types (type II, V, and VI) and 17 subtypes of systems, including the RNA-guided Dnases Cas9 and Cas12 and the RNA-guided Rnase Cas13. Continual sequencing of novel bacterial genomes and metagenomes uncovers new diversity of CRISPR-Cas systems and their evolutionary relationships, necessitating experimental work that reveals the function of these systems and develops them into new tools.

Among the currently known CRISPR-Cas systems, only the type III and type VI systems have been demonstrated to bind and target RNA, and these two systems have substantially different properties, the most distinguishing being their membership in Class 1 and Class 2, respectively. Characterized subtypes of type III, which span type III-A, B, and C systems, target both RNA and DNA species through an effector complex containing multiple Cas7 (Csm3/5 or Cmr1/4/6) RNA nuclease units in association with a single Cas10 (Csm1 or Cmr2) DNA nuclease. The RNA nuclease activity of Cas7 is mediated through acidic residues in the repeat-associated mysterious proteins (RAMP) domains, which cut at stereotyped intervals in the guide:target duplex. Type III systems also have a target restriction and cannot efficiently target protospacers in vivo if there is extended homology between the 5′ “tag” of the crRNA and the “anti-tag” 3′ of the protospacer in the target, although this binding does not block RNA cleavage in vitro. In type III systems, pre-crRNA processing is carried out by either host factors or the associated Cas6 family protein, which can physically complex with the effector machinery.

In contrast to type III systems, type VI systems contain a single CRISPR effector Cas13 that can only effect RNA interference, mediated through basic catalytic residues of dual HEPN domains. This interference requires a protospacer flanking sequence (PFS), although the influence of the PFS varies between orthologs and families. Importantly, the RNA cleavage activity of Cas13, once triggered by crRNA:target duplex formation, is indiscriminate, and activated Cas13 enzymes will cleave other RNA species in vitro, in bacterial hosts, and mammalian cells. This activity, termed the collateral effect, has been applied to CRISPR-based nucleic acid detection technologies. In addition to the RNA interference activity, the Cas13 family members contain pre-crRNA processing activity. Just as single-effector DNA targeting systems have given rise to numerous genome editing applications, Cas13 family members have been applied to a suite of RNA-targeting technologies in both bacterial and eukaryotic cells, including RNA knockdown, RNA editing, RNA tracking, epitranscriptome editing, translational upregulation, epi-transcriptomic reading and writing via N6-Methyladenosine, and isoform modulation.

The novel type III-E system was recently identified from genomes of 8 bacterial species and is characterized as a fusion of several Cas7 proteins and a putative Cas11 (Csm2)-like small subunit. The domain composition suggests the fusion of multiple type III effector module domains involved in crRNA binding into a single protein effector that is predicted to process pre-crRNA given its homology with Cas5 (Csm4) and conserved aspartates. The lack of other putative effector nucleases in these CRISPR loci raise the additional possibility that this fusion protein is capable of crRNA-directed RNA cleavage. If so, this system would blur the distinction of Class 1 and Class 2 systems, as it would have domains homologous to other Class 1 systems and possess a single effector module characteristic of Class 2 systems. Beyond the single effector module present in all subtype III-E loci, a majority of type III-E family members contain a putative ancillary gene with a CHAT domain, which is a caspase family protease associated with programmed cell death (PCD), suggesting involvement of PCD-mediated antiviral strategies, as has been observed with type III and VI systems.

Type III-E system associated effector is a programmable Rnase. This system can provide defense against RNA phage and be programmed to target exogenous mRNA species when expressed heterologously in bacteria. Orthologs of Cas7-11 are capable of both processing of pre-crRNA and crRNA-directed cleavage of RNA targets and determine catalytic residues underlying programmed RNA cleavage. A direct evolutionary path of Cas7-11 can be traced from individual Cas7 and Cas11 effector proteins of subtype III-D1 variant, through an intermediate, a partially fused effector Cas7×3 of the subtype III-D2 variant, to the singe-effector architecture of subtype III-E that is so far unique among the Class 1 CRISPR-Cas systems. Cas7-11 most likely originated from two type III-D variants. Three Cas7 domains (domains 3, 4 and 5) are derived from subtype III-D2 that contains a the Cas7×3 effector protein along with Cas10 and another Cas7-like domain fused to a Cas5-like domain. The origin of the N-terminal Cas7 and putative Cas11 domain of Cas7-11 is most likely derived from a III-D1 variant, where both genes are stand-alone.

Cas7-11 differs from Cas13, in terms of both domain organization and activity. Cas13 RNA cleavage is enacted by dual HEPN domains with basic catalytic residues, and this cleavage, once triggered, is indiscriminate. In contrast, Cas7-11 utilizes at least two of four Cas7-like domains with acidic catalytic residues to generate stereotyped cleavage at the target binding site in cis. Furthermore, Cas13 targeting is restricted by the requirement for a PFS, which Cas7-11 does not require, and the DR of Cas7-11-associated crRNA is substantially shorter. Because of these unique features, Cas7-11 may have distinct advantages for RNA targeting and transcriptome engineering biotechnology applications.

Regulation of interference by accessory proteins has been observed in both type III and type VI systems, and other proteins in the D. ishimotonii type III-E locus can regulate activity of DiCas7-11a. Notably, TPR-CHAT had a strong inhibitory effect on DiCas7-11a phage interference, raising the possibility that unrestricted DiCas7-11a activity could be detrimental for the host. Alternatively, as TPR-CHAT is a caspase family protease associated with programmed cell death (PCD), it is possible that TPR-CHAT is activated by DiCas7-11a and leads to host death, which could mimic death due to phage in these assays. TPR-CHAT caspase activity could be activated by DiCas7-11a and cause PCD through general proteolysis, analogous to PCD triggered by Cas13 collateral activity.

Similar to Class 2 CRISPR effectors such as Cas9, Cas12, and Cas13, Cas7-11 is highly active in mammalian cells, with substantial knockdown activity on both reporter and endogenous transcripts. Moreover, via inactivation of active sites through mutagenesis, the catalytically inactive dCas7-11 enzyme can be used to recruit ADAR2DD for efficient site-specific A-to-I editing on transcripts. These applications establish Cas7-11 as the basis for an RNA-targeting toolbox that has several benefits compared to Cas13, including the lack of sequence preferences and collateral activity, the latter of which has been shown to induce toxicity in certain cell types. A Cas7-11 toolbox may serve as the basis for multiple RNA technologies, including RNA knockdown, RNA editing, translation modulation, RNA recruitment, RNA tracking, splicing control, RNA stabilization, and potentially even diagnostics.

AD-Functionalized CRISPR Systems

In some embodiments, the systems may be AD-functionalized CRISPR system. The term “AD-functionalized CRISPR system” as used here refers to a nucleic acid targeting and editing system comprising (a) a CRISPR-Cas protein, more particularly a Cas7-11 protein which is catalytically active or inactive; (b) a guide molecule which comprises a guide sequence; and (c) an adenosine deaminase (AD) protein or catalytic domain thereof; wherein the adenosine deaminase protein or catalytic domain thereof is covalently or non-covalently linked to the CRISPR-Cas protein or the guide molecule or is adapted to link thereto after delivery; wherein the guide sequence is substantially complementary to the target sequence but comprises a non-pairing C corresponding to the A being targeted for deamination, resulting in an A-C mismatch in an RNA duplex formed by the guide sequence and the target sequence. In some embodiments, the CRISPR-Cas protein and/or the adenosine deaminase comprise one or more heterologous nuclear export signal(s) (NES(s)) or nuclear localization signal(s)(NLS(s)). For application in eukaryotic cells, the CRISPR-Cas protein and/or the adenosine deaminase can be NES-tagged or NLS-tagged.

One skilled in the art would appreciate that the components (a), (b) and (c) can be delivered to the cell as a ribonucleoprotein complex. The ribonucleoprotein complex can be delivered via one or more lipid nanoparticles. One skilled in the art would appreciate that the components (a), (b) and (c) can be delivered to the cell as one or more RNA molecules, such as one or more guide RNAs and one or more mRNA molecules encoding the CRISPR-Cas protein, the adenosine deaminase protein, and optionally the adaptor protein. The RNA molecules can be delivered via one or more lipid nanoparticles. One skilled in the art would appreciate that the components (a), (b) and (c) can be delivered to the cell as one or more DNA molecules. The one or more DNA molecules can be comprised within one or more vectors such as viral vectors (e.g., AAV). The one or more DNA molecules can comprise one or more regulatory elements operably configured to express the CRISPR-Cas protein, the guide molecule, and the adenosine deaminase protein or catalytic domain thereof, optionally wherein the one or more regulatory elements comprise inducible promoters.

In some embodiments, the CRISPR-Cas protein is a dead Cas7-11. In some embodiments, the dead Cas7-11 comprises one or more mutations in the Cas7-like domains, including D429A and D654A as well as many other mutations.

In some embodiments, the CRISPR-Cas protein is a Cas7-11 endonuclease with an amino acid sequence comprising at least 1 mutation or modification, at least 2 mutations or modifications, at least 3 mutations or modifications, at least 4 mutations or modifications, at least 5 mutations or modifications, at least 6 mutations or modifications, at least 7 mutations or modifications, at least 8 mutations or modifications, at least 9 mutations or modifications, at least 10 mutations or modifications, or any ranges that are made of any two or more points in the above list of mutations or modifications. In some embodiments, the Cas7-11 endonuclease is a DiCas7-11 endonuclease.

In some embodiments, the guide molecule is capable of hybridizing with a target sequence comprising the Adenine to be deaminated within an RNA sequence to form an RNA duplex which comprises a non-pairing Cytosine opposite to said Adenine. Upon RNA duplex formation, the guide molecule forms a complex with the Cas7-11 protein and directs the complex to bind the RNA polynucleotide at the target RNA sequence of interest. Details on the aspect of the guide of the AD-functionalized CRISPR-Cas system are provided herein below.

In at least a first design, the AD-functionalized CRISPR system comprises: (a) an adenosine deaminase fused or linked to a CRISPR-Cas protein, wherein the CRISPR-Cas protein is catalytically inactive; and (b) a guide molecule comprising a guide sequence designed to introduce an A-C mismatch in an RNA duplex formed between the guide sequence and the target sequence. In some embodiments, the CRISPR-Cas protein and/or the adenosine deaminase can be NLS-tagged on either the N- or C-terminus or both.

In at least a second design, the AD-functionalized CRISPR system comprises: (a) a CRISPR-Cas protein that is catalytically inactive; (b) a guide molecule comprising a guide sequence designed to introduce an A-C mismatch in an RNA duplex formed between the guide sequence and the target sequence, and an aptamer sequence (e.g., MS2 RNA motif or PP7 RNA motif) capable of binding to an adaptor protein (e.g., MS2 coating protein or PP7 coat protein); and (c) an adenosine deaminase fused or linked to an adaptor protein, wherein the binding of the aptamer and the adaptor protein recruits the adenosine deaminase to the RNA duplex formed between the guide sequence and the target sequence for targeted deamination at the A of the A-C mismatch. In some embodiments, the adaptor protein and/or the adenosine deaminase can be NLS-tagged on either the N- or C-terminus or both. The CRISPR-Cas protein can also be NLS-tagged. The CRISPR-Cas protein can also be NLS-tagged.

The use of different aptamers and corresponding adaptor proteins also allows orthogonal gene editing to be implemented. In one example in which adenosine deaminase are used in combination with cytidine deaminase for orthogonal gene editing/deamination, sgRNA targeting different loci are modified with distinct RNA loops in order to recruit MS2-adenosine deaminase and PP7-cytidine deaminase (or PP7-adenosine deaminase and MS2-cytidine deaminase), respectively, resulting in orthogonal deamination of A or C at the target loci of interested, respectively. PP7 is the RNA-binding coat protein of the bacteriophage Pseudomonas. Like MS2, it binds a specific RNA sequence and secondary structure. The PP7 RNA-recognition motif is distinct from that of MS2. Consequently, PP7 and MS2 can be multiplexed to mediate distinct effects at different genomic loci simultaneously. For example, an sgRNA targeting locus A can be modified with MS2 loops, recruiting MS2-adenosine deaminase, while another sgRNA targeting locus B can be modified with PP7 loops, recruiting PP7-cytidine deaminase. In the same cell, orthogonal, locus-specific modifications are thus realized. This principle can be extended to incorporate other orthogonal RNA-binding proteins.

In at least a third design, the AD-functionalized CRISPR system comprises: (a) an adenosine deaminase inserted into an internal loop or unstructured region of a CRISPR-Cas protein, wherein the CRISPR-Cas protein is catalytically inactive or a nickase; and (b) a guide molecule comprising a guide sequence designed to introduce an A-C mismatch in an RNA duplex formed between the guide sequence and the target sequence.

The AD-functionalized CRISPR system described herein can be used to target a specific Adenine within an RNA polynucleotide sequence for deamination. For example, the guide molecule can form a complex with the CRISPR-Cas protein and directs the complex to bind a target RNA sequence in the RNA polynucleotide of interest. Because the guide sequence is designed to have a non-pairing C, the RNA duplex formed between the guide sequence and the target sequence comprises an A-C mismatch, which directs the adenosine deaminase to contact and deaminate the A opposite to the non-pairing C, converting it to an Inosine (I). Since Inosine (I) base pairs with C and functions like G in cellular processes, the targeted deamination of A described herein are useful for correction of undesirable G-A and C-T mutations, as well as for obtaining desirable A-G and T-C mutations.

In some embodiments, the AD-functionalized CRISPR system is used for targeted deamination in an RNA polynucleotide molecule in vitro. In some embodiments, the AD-functionalized CRISPR system is used for targeted deamination in a DNA molecule and/or RNA molecule within a cell. The cell can be a eukaryotic cell such as a bacteria or cyanobacteria. The cell can be a eukaryotic cell, such as an animal cell, a mammalian cell, a human, or a plant cell.

The disclosure also relates to a (non-naturally occurring or engineered) method for treating or preventing a disease by the targeted deamination using the AD-functionalized CRISPR system, wherein the deamination of the A, which remedies a disease caused by transcripts containing a pathogenic G→A or C→T point mutation. Examples of disease that can be treated or prevented with the present disclosure include cancer, Meier-Gorlin syndrome, Seckel syndrome 4, Joubert syndrome 5, Leber congenital amaurosis 10; Charcot-Marie-Tooth disease, type 2; Charcot-Marie-Tooth disease, type 2; Usher syndrome, type 2C; Spinocerebellar ataxia 28; Spinocerebellar ataxia 28; Spinocerebellar ataxia 28; Long QT syndrome 2; Sjogren-Larsson syndrome; Hereditary fructosuria; Hereditary fructosuria; Neuroblastoma; Neuroblastoma; Kallmann syndrome 1; Kallmann syndrome 1; Kallmann syndrome 1; Metachromatic leukodystrophy.

AD-functionalized CRISPR system for RNA editing can be used for translation upregulation or downregulation, improving RNA stability and diagnostics. For example, for application in diagnostics, TPR-Chat is an accessory protein that interacts with Cas7-11 interference. Cas7-11 can activate TPR-Chat caspase activity which can then activate a reporter. While this can be used for inducing cell death based on RNA detection (e.g., in cancer cells), it also can be useful for general RNA diagnostics (i.e., molecular diagnostics for bacteria, viruses, and derivatives thereof) in samples. Furthermore, Cas7-11 can re-constitute a split protein like GFP on a specific transcript.

AD-functionalized CRISPR system for RNA editing can be used to treat or prevent premature termination diseases. Pre-termination diseases are characterized by mutations in early stop codons, either through single nucleotide polymorphisms that introduce termination, indels that change the translational frame of the protein and generate new stop codons, or alternative splicing that preferentially introduces exons that have early termination. By removing stop codons generated in these ways via A to I editing, RNA editing with ADAR could rescue diseases involving premature termination. In cases where SNPs are not G to A, but generate nonsense mutations, clinical benefit could be derived from changing nonsense mutations into missense mutations.

AD-functionalized CRISPR system for RNA editing can be used to change fertility mutations without germline editing. One advantage of RNA editing over DNA editing is in cases of SNPs affecting fertility, where correction with genome editing would necessarily result in germline editing, with potential ethical or safety implications. RNA editing could correct these mutations without permanent effects on the genome, thereby circumventing these issues.

AD-functionalized CRISPR system for RNA editing can be used for splicing alteration. Pre-mRNA requires specific splice donor and acceptor sequences in order to undergo processing by the spliceosome. Splice acceptor sites contain an invariant AG sequence that is necessary for acceptance of the attack by the splice donor sequence and intron removal. By targeting Cas7-11-ADAR fusions to pre-mRNA and editing AG splice acceptor sites to IG, it can be possible to inactivate the splice acceptor site, resulting in skipping of the downstream exon. This approach to splicing alteration has advantages over the current method of exon skipping with chemically modified anti-sense oligos. Cas7-11-ADAR can be genetically encoded, allowing for long-term exon skipping. Additionally, Cas7-11-ADAR creates a mutation to promote skipping, which can be more robust than masking of the splice donor/acceptor site by a double stranded RNA, as is done with anti-sense oligos.

AD-functionalized CRISPR system for RNA editing can be used to alter neoantigens. Neoantigens in cancer are novel antigens that are expressed in tumor cells due to mutations that arise because of defective mismatch repair. Engineering T cells against neoantigens is advantageous because the T cells will have no off-target activity and thus toxicity since the antigens are only expressed in the tumor cells. With RNA base editors, the Cas7-11-ADAR fusions can be targeted to cancer cells to introduce mutations in transcripts that would introduce amino acid changes and new antigens that can be targeted using chimeric antigen receptor T cells. This approach is better than DNA base editors because it is transient and thus the risk of editing non-tumor cells permanently due to off-target delivery is minimal.

AD-functionalized CRISPR system for RNA editing can be used to change microRNA targets for tumor suppressors. ADAR naturally edits mRNA to generate or remove microRNA targets, thereby modulating expression. Programmable RNA editing can be used to up- or down-regulate microRNA targets via altering of targeting regions. Additionally, microRNAs themselves are natural substrates for ADAR, and programmable RNA editing of microRNAs can reduce or enhance the function on their corresponding targets.

AD-functionalized CRISPR system for RNA editing can be used to make multiple edits along a region. The Cas7-11-ADAR fusions can be precisely targeted to edit specific adenosines by introducing a mismatch in the guide region across from the desired adenosine target and creating a bubble that is favorable for A-to-I editing. By introducing multiple of these mismatches across different adenosine sites in the guide/target duplex, it can be possible to introduce multiple mutations at once.

AD-functionalized CRISPR system for RNA editing can be used for the reversal of TAA (double A to G) for PTC. Many diseases that involve pretermination codon changes involve a TAA stop codon, which would require A-to-I changes to correct rather than the TAG or TGA stop codons which only need one A-to-I edit. Two approaches can be used to reverse the TAA stop codon. (1) As described in the previous section, two mismatches can be introduced in the guide against the two adenosines in the TAA codon. (2) A two-guide array can be used to convert each of the adenosines to inosine sequentially. The first guide in the array can contain a mutation against the first adenosine and the second guide can then have complementarity to this change and have a mismatch against the second adenosine in the stop codon.

AD-functionalized CRISPR system for RNA editing can be used to treat or prevent cancer (GOF, LOF mutation reversal). Many oncogenic changes in cancer involve G to A mutations that introduce gain of function or loss of function phenotypes to the mutated proteins. The RNA base editors are well positioned to correct these changes and reduce oncogenesis.

RNA editing with ADAR can be used for the design of new base preferences. Current ADAR1/2 proteins have been found to have surrounding base preferences for catalytic activity, which may pose constraints for certain applications. Rational mutagenesis or directed evolution of ADAR variants with altered or relaxed base preferences can increase the versatility of programmable RNA editing.

AD-functionalized CRISPR system for RNA editing can comprise ADAR mutants with increased activity in human cells. Although ADAR mutants with altered activity in vitro or in yeast have been previously reported, screening or rational design of mutants with increased activity in the context of human cells can improve the efficiency or specificity of ADAR-based programmable RNA editing constructs.

AD-functionalized CRISPR system for RNA editing can be used in biological applications of inosine generation. The RNA editing with ADAR generates inosine, which, when occurring multiple times in a transcript, can interact with endogenous biological pathways to increase inflammation in cells and tissues. Generation of multiple inosine bases can increase inflammation, especially in cells where inflammation can lead to clearance. Additional inosine generation could also be used to destabilize transcripts.

AD-functionalized CRISPR system for RNA editing can be used in removing upstream start codons to promote protein expression of downstream ORF (ATG mutation). Anti-sense oligos have been used for blocking upstream start codon sites to promote protein expression at downstream start codons. This allows the boosting of endogenous protein levels for therapeutic purposes. Cas7-11-ADAR fusions could accomplish a similar effect by converting ATG sites to ITG (GTG) sites and thus remove upstream codons in endogenous transcripts and thus boost protein translation. So far, most therapeutic applications discussed have been for correcting G to A mutations or removing pre-termination sites. This would be an application that allows for boosting gene expression. A good example is boosting fetal hemoglobin levels in sickle cell disease and thalassemias.

AD-functionalized CRISPR system for RNA editing can comprise the mutagenesis of ADAR for C to U or any transition. It is possible through rational mutagenesis or directed evolution that the ADARs listed in the ortholog section could be made into C to U editors or editors of any base transition.

In particular embodiments, the compositions described herein can be used in therapy. This implies that the methods can be performed in vivo, ex vivo or in vitro. In particular embodiments, the methods can be not methods of treatment of the animal or human body or a method for modifying the germ line genetic identity of a human cell. In particular embodiments, when carrying out the method, the target RNA can be not comprised within a human or animal cell. In particular embodiments, when the target is a human or animal target, the method can be carried out ex vivo or in vitro.

CRISPR-Cas Proteins and Guides

In some embodiments, the system comprises one or more components of a CRISPR-Cas system. For example, the system may comprise a Cas protein, a guide molecule, or a combination thereof.

In the methods and systems of the present disclosure use is made of a CRISPR-Cas protein and corresponding guide molecule. More particularly, the CRISPR-Cas protein is a class 2 CRISPR-Cas protein. In certain embodiments, said CRISPR-Cas protein is a Cas7-11. The Cas7-11 may be Cas7-11a, Cas7-11b, Cas7-11c, or Cas7-11d. The CRISPR-Cas system does not require the generation of customized proteins to target specific sequences but rather a single Cas protein can be programmed by guide molecule to recognize a specific nucleic acid target, in other words the Cas enzyme protein can be recruited to a specific nucleic acid target locus of interest using said guide molecule.

CRISPR-Cas Proteins

In some embodiments, the systems may comprise a CRISPR-Cas protein. In certain examples, the CRISPR-Cas protein may be a catalytically inactive (dead) Cas protein. The catalytically inactive (dead) Cas protein may have impaired (e.g., reduced or no) nuclease activity. In some cases, the dead Cas protein may have nickase activity. In some cases, the dead Cas protein may be dead Cas 15 protein. For example, the dead Cas 15 may be dead Cas7-11a, dead Cas7-11b, dead Cas7-11c, or dead Cas7-11d. In some embodiments, the system may comprise a nucleotide sequence encoding the dead Cas protein.

In its unmodified form, a CRISPR-Cas protein is a catalytically active protein. This implies that upon formation of a nucleic acid-targeting complex (comprising a guide RNA hybridized to a target sequence) one or both DNA strands in or near (e.g., within 1, 2, 3, 4, 5, 6, 7, 8, 9, 10, 20, 50, or more base pairs from) the target sequence is modified (e.g., cleaved). As used herein the term “sequence(s) associated with a target locus of interest” refers to sequences near the vicinity of the target sequence (e.g., within 1, 2, 3, 4, 5, 6, 7, 8, 9, 10, 20, 50, or more base pairs from the target sequence, wherein the target sequence is comprised within a target locus of interest). The unmodified catalytically active Cas7-11 protein generates a staggered cut, whereby “the cut sites are typically within the target sequence” More particularly, the staggered cut is typically 13-23 nucleotides distal to the PAM. In particular embodiments, the cut on the non-target strand is 17 nucleotides downstream of the PAM (i.e. between nucleotide 17 and 18 downstream of the PAM), while the cut on the target strand (i.e. strand hybridizing with the guide sequence) occurs a further 4 nucleotides further from the sequence complementary to the PAM (this is 21 nucleotides upstream of the complement of the PAM on the 3′ strand or between nucleotide 21 and 22 upstream of the complement of the PAM).

In the methods according to the present disclosure, the CRISPR-Cas protein is mutated with respect to a corresponding wild-type enzyme such that the mutated CRISPR-Cas protein lacks the ability to cleave one or both DNA strands of a target locus containing a target sequence. In particular embodiments, one or more catalytic domains of the Cas7-11 protein are mutated to produce a mutated Cas protein which cleaves only one DNA strand of a target sequence.

In particular embodiments, the CRISPR-Cas protein may be mutated with respect to a corresponding wild-type enzyme such that the mutated CRISPR-Cas protein lacks substantially all DNA cleavage activity. In some embodiments, a CRISPR-Cas protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form.

In certain embodiments of the methods provided herein the CRISPR-Cas protein is a mutated CRISPR-Cas protein which cleaves only one DNA strand, i.e., a nickase. More particularly, in the context of the present disclosure, the nickase ensures cleavage within the non-target sequence, i.e., the sequence which is on the opposite DNA strand of the target sequence and 3′ of the PAM sequence.

In some embodiments, a CRISPR-Cas protein is considered to substantially lack all DNA cleavage activity when the DNA cleavage activity of the mutated enzyme is about no more than 25%, 10%, 5%, 1%, 0.1%, 0.01%, or less of the DNA cleavage activity of the non-mutated form of the enzyme; an example can be when the DNA cleavage activity of the mutated form is nil or negligible as compared with the non-mutated form. In these embodiments, the CRISPR-Cas protein is used as a generic DNA binding protein. The mutations may be artificially introduced mutations or gain- or loss-of-function mutations.

In addition to the mutations described above, the CRISPR-Cas protein may be additionally modified. As used herein, the term “modified” with regard to a CRISPR-Cas protein generally refers to a CRISPR-Cas protein having one or more modifications or mutations (including point mutations, truncations, insertions, deletions, chimeras, fusion proteins, etc.) compared to the wild type Cas protein from which it is derived. A modification by truncation can refer to an engineered truncation that is based on structure function analysis and not naturally occurring. By derived is meant that the derived enzyme is largely based, in the sense of having a high degree of sequence homology with, a wildtype enzyme, but that it has been mutated (modified) in some way as known in the art or as described herein. The modification can be fusions of effectors like fluorophore, proteins involved in translation modulation (e.g., eIF4E, eIF4A, and eIF4G) and proteins involved with epitranscriptomic modulation (e.g., pseudouridine synthase and m6a writer/readers), and splicing factors involved with changing splicing. Cas7-11 could also be used for sensing RNA for diagnostic purposes.

In some embodiments, the C-terminus of the Cas7-11 effector can be truncated. For example, at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 15 amino acids, at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, at least 300 amino acids, at least 350 amino acids, or any ranges that are made of any two or more points in the above list may be truncated at the C-terminus of the Cas7-11 effector. For example, up to 120 amino acids, up to 140 amino acids, up to 160 amino acids, up to 180 amino acids, up to 200 amino acids, up to 250 amino acids, up to 300 amino acids, up to 350 amino acids, up to 400 amino acids, or any ranges that are made of any two or more points in the above list may be truncated at the C-terminus of the Cas7-11 effector.

In some embodiments, the N-terminus of the Cas7-11 effector protein may be truncated. For example, at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 15 amino acids, at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, at least 300 amino acids, at least 350 amino acids, or any ranges that are made of any two or more points in the above list may be truncated at the N-terminus of the Cas7-11 effector. For examples, up to 120 amino acids, up to 140 amino acids, up to 160 amino acids, up to 180 amino acids, up to 200 amino acids, up to 250 amino acids, up to 300 amino acids, up to 350 amino acids, up to 400 amino acids, or any ranges that are made of any two or more points in the above list may be truncated at the N-terminus of the Cas7-11 effector.

In some embodiments, both the N- and the C-termini of the Cas7-11 effector protein may be truncated. For example, at least 20 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 40 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 60 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 80 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 100 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 120 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 140 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 160 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 180 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 200 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 220 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 240 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 260 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 280 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 300 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 350 amino acids may be truncated at the C-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas7-11 effector. For example, at least 20 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 40 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 60 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 80 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 100 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 120 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 140 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 160 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 180 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 200 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 220 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 240 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 260 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 280 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 300 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector. For example, at least 350 amino acids may be truncated at the N-terminus of the Cas7-11 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas7-11 effector.

In some embodiments, the Cas7-11 effector comprises a deletion of the INS domain. For example, at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 15 amino acids, at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, at least 300 amino acids, at least 350 amino acids, or any ranges that are made of any two or more points in the above list of the INS domain may be deleted.

In some embodiments, the INS domain of the Cas7-11 effector is replaced by a linker. See, e.g., Reddy Chichili, V. P., Kumar, V., & Sivaraman, J., “Linkers in the structural biology of protein-protein interactions,” Protein science: a publication of the Protein Society, 22(2), 153-167 (2013); https//doi.org/10.1002/pro.2206, incorporated herewith in its entirety by reference. For example, the INS domain of the Cas7-11 effector may be replaced by a GG, GGG, GS, GGS, GGGS(SEQ ID NO:77), and/or GGGGS(SEQ ID NO:78) linker. For example, the INS domain of the Cas7-11 effector may be replaced by a (GG)x(SEQ ID NO:114), (GGG)x(SEQ ID NO:115), (GGS)x(SEQ ID NO:116), (GGGS)x(SEQ ID NO:117), and/or a (GGGGS)x(SEQ ID NO:118) linker, wherein x is independently 1, 2, 3, 4, 5, 6, 7, 8, 9, 10, 11, or 12. For example, the INS domain of the Cas7-11 effector may be replaced by a linker with at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, or any ranges that are made of any two or more points in the above list.

The additional modifications of the CRISPR-Cas protein may or may not cause an altered functionality. By means of example, and in particular with reference to CRISPR-Cas protein, modifications which do not result in an altered functionality include for instance codon optimization for expression into a particular host or providing the nuclease with a particular marker (e.g., for visualization). Modifications with may result in altered functionality may also include mutations, including point mutations, insertions, deletions, truncations (including split nucleases), etc. Fusion proteins may without limitation include for instance fusions with heterologous domains or functional domains (e.g., localization signals, catalytic domains, etc.). In certain embodiments, various modifications may be combined (e.g., a mutated nuclease which is catalytically inactive, and which further is fused to a functional domain, such as for instance to induce DNA methylation or another nucleic acid modification, such as including without limitation a break (e.g., by a different nuclease (domain)), a mutation, a deletion, an insertion, a replacement, a ligation, a digestion, a break or a recombination). As used herein, “altered functionality” includes without limitation an altered specificity (e.g., altered target recognition, increased (e.g., “enhanced” Cas proteins) or decreased specificity, or altered PAM recognition), altered activity (e.g., increased or decreased catalytic activity, including catalytically inactive nucleases or nickases), and/or altered stability (e.g., fusions with destabilization domains). Suitable heterologous domains include without limitation a nuclease, a ligase, a repair protein, a methyltransferase, (viral) integrase, a recombinase, a transposase, an argonaute, a cytidine deaminase, a retron, a group II intron, a phosphatase, a phosphorylase, a sulfurylase, a kinase, a polymerase, an exonuclease, etc. Examples of all these modifications are known in the art. It will be understood that a “modified” nuclease as referred to herein, and in particular a “modified” Cas or “modified” CRISPR-Cas system or complex preferably still has the capacity to interact with or bind to the poly-nucleic acid (e.g., in complex with the guide molecule). Such modified Cas protein can be combined with the deaminase protein or active domain thereof as described herein.

In certain embodiments, CRISPR-Cas protein may comprise one or more modifications resulting in enhanced activity and/or specificity, such as including mutating residues that stabilize the targeted or non-targeted strand (e.g., eCas9; “Rationally engineered Cas9 nucleases with improved specificity”, Slaymaker et al. (2016), Science, 351(6268):84-88, incorporated herewith in its entirety by reference). In certain embodiments, the altered or modified activity of the engineered CRISPR protein comprises increased targeting efficiency or decreased off-target binding. In certain embodiments, the altered activity of the engineered CRISPR protein comprises modified cleavage activity. In certain embodiments, the altered activity comprises increased cleavage activity as to the target polynucleotide loci. In certain embodiments, the altered activity comprises decreased cleavage activity as to the target polynucleotide loci. In certain embodiments, the altered activity comprises decreased cleavage activity as to off-target polynucleotide loci. In certain embodiments, the altered or modified activity of the modified nuclease comprises altered helicase kinetics. In certain embodiments, the modified nuclease comprises a modification that alters association of the protein with the nucleic acid molecule comprising RNA (in the case of a Cas protein), or a strand of the target polynucleotide loci, or a strand of off-target polynucleotide loci. In an aspect of the disclosure, the engineered CRISPR protein comprises a modification that alters formation of the CRISPR complex. In certain embodiments, the altered activity comprises increased cleavage activity as to off-target polynucleotide loci. Accordingly, in certain embodiments, there is increased specificity for target polynucleotide loci as compared to off-target polynucleotide loci. In other embodiments, there is reduced specificity for target polynucleotide loci as compared to off-target polynucleotide loci. In certain embodiments, the mutations result in decreased off-target effects (e.g., cleavage or binding properties, activity, or kinetics), such as in case for Cas proteins for instance resulting in a lower tolerance for mismatches between target and guide RNA. Other mutations may lead to increased off-target effects (e.g., cleavage or binding properties, activity, or kinetics). Other mutations may lead to increased or decreased on-target effects (e.g., cleavage or binding properties, activity, or kinetics). In certain embodiments, the mutations result in altered (e.g., increased or decreased) helicase activity, association, or formation of the functional nuclease complex (e.g., CRISPR-Cas complex). In certain embodiments, as described above, the mutations result in an altered PAM recognition, i.e., a different PAM may be (in addition or in the alternative) be recognized, compared to the unmodified Cas protein. Particularly preferred mutations include positively charged residues and/or (evolutionary) conserved residues, such as conserved positively charged residues, in order to enhance specificity. In certain embodiments, such residues may be mutated to uncharged residues, such as alanine. In certain embodiments, such residues may be mutated to charged residues, such as arginine and lysine.

Type-III CRISPR-Cas Proteins

The application describes methods using Type-III CRISPR-Cas proteins. This is exemplified herein with Cas7-11, whereby a number of orthologs or homologs have been identified. It will be apparent to the skilled person that further orthologs or homologs can be identified and that any of the functionalities described herein may be engineered into other orthologs, including chimeric enzymes comprising fragments from multiple orthologs.

Computational methods of identifying novel CRISPR-Cas loci are described in EP3009511 or US2016208243 and may comprise the following steps: detecting all contigs encoding the Cas1 protein; identifying all predicted protein coding genes within 20 kB of the cas1 gene; comparing the identified genes with Cas protein-specific profiles and predicting CRISPR arrays; selecting unclassified candidate CRISPR-Cas loci containing proteins larger than 500 amino acids (>500 aa); analyzing selected candidates using methods such as PSI-BLAST and HHPred to screen for known protein domains, thereby identifying novel Class 2 CRISPR-Cas loci (see also Schmakov et al. 2015, Mol Cell. 60(3):385-97). In addition to the above-mentioned steps, additional analysis of the candidates may be conducted by searching metagenomics databases for additional homologs. Additionally, or alternatively, to expand the search to non-autonomous CRISPR-Cas systems, the same procedure can be performed with the CRISPR array used as the seed.

In one aspect the detecting all contigs encoding the Cas1 protein is performed by GenemarkS, a gene prediction program as further described in “GeneMarkS: a self-training method for prediction of gene starts in microbial genomes. Implications for finding sequence motifs in regulatory regions.” John Besemer, Alexandre Lomsadze and Mark Borodovsky, Nucleic Acids Research (2001) 29, pp 2607-2618, herein incorporated by reference.

In one aspect the identifying all predicted protein coding genes is carried out by comparing the identified genes with Cas protein-specific profiles and annotating them according to NCBI Conserved Domain Database (CDD) which is a protein annotation resource that consists of a collection of well-annotated multiple sequence alignment models for ancient domains and full-length proteins. These are available as position-specific score matrices (PSSMs) for fast identification of conserved domains in protein sequences via RPS-BLAST. CDD content includes NCBI-curated domains, which use 3D-structure information to explicitly define domain boundaries and provide insights into sequence/structure/function relationships, as well as domain models imported from a number of external source databases (Pfam, SMART, COG, PRK, TIGRFAM). In a further aspect, CRISPR arrays were predicted using a PILER-CR program which is a public domain software for finding CRISPR repeats as described in “PILER-CR: fast and accurate identification of CRISPR repeats,” Edgar, R. C., BMC Bioinformatics, Jan 20; 8:18(2007), herein incorporated by reference.

In a further aspect, the case-by-case analysis is performed using PSI-BLAST (Position-Specific Iterative Basic Local Alignment Search Tool). PSI-BLAST derives a position-specific scoring matrix (PSSM) or profile from the multiple sequence alignment of sequences detected above a given score threshold using protein-protein BLAST. This PSSM is used to further search the database for new matches and updated for subsequent iterations with these newly detected sequences. Thus, PSI-BLAST provides a means of detecting distant relationships between proteins.

In another aspect, the case-by-case analysis is performed using Hhpred, a method for sequence database searching and structure prediction that is as easy to use as BLAST or PSI-BLAST and that is at the same time much more sensitive in finding remote homologs. In fact, Hhpred's sensitivity is competitive with the most powerful servers for structure prediction currently available. Hhpred is the first server that is based on the pairwise comparison of profile hidden Markov models (HMMs). Whereas most conventional sequence search methods search sequence databases such as UniProt or the NR, Hhpred searches alignment databases, like Pfam or SMART. This greatly simplifies the list of hits to a number of sequence families instead of a clutter of single sequences. All major publicly available profile and alignment databases are available through Hhpred. Hhpred accepts a single query sequence or a multiple alignment as input. Within only a few minutes it returns the search results in an easy-to-read format similar to that of PSI-BLAST. Search options include local or global alignment and scoring secondary structure similarity. Hhpred can produce pairwise query-template sequence alignments, merged query-template multiple alignments (e.g., for transitive searches), as well as 3D structural models calculated by the MODELLER software from Hhpred alignments.

Deactivated/Inactivated Cas7-11 Proteins

Where the Cas7-11 protein has nuclease activity, the Cas7-11 protein may be modified to have diminished nuclease activity e.g., nuclease inactivation of at least 70%, at least 80%, at least 90%, at least 95%, at least 97%, or 100% as compared with the wild type enzyme; or to put in another way, a Cas7-11 enzyme having advantageously about 0% of the nuclease activity of the non-mutated or wild type Cas7-11 enzyme or CRISPR-Cas protein, or no more than about 3% or about 5% or about 10% of the nuclease activity of the non-mutated or wild type Cas7-11 enzyme.

Modified Cas7-11 enzymes

In particular embodiments, it is of interest to make use of an engineered Cas7-11 protein as defined herein, such as Cas7-11, wherein the protein complexes with a nucleic acid molecule comprising RNA to form a CRISPR complex, wherein when in the CRISPR complex, the nucleic acid molecule targets one or more target polynucleotide loci, the protein comprises at least one modification compared to unmodified Cas7-11 protein, and wherein the CRISPR complex comprising the modified protein has altered activity as compared to the complex comprising the unmodified Cas7-11 protein. It is to be understood that when referring herein to CRISPR “protein,” the Cas7-11 protein is an unmodified or modified CRISPR-Cas protein (e.g., having increased or decreased or the same (or no) enzymatic activity, such as without limitation including Cas7-11. The term “CRISPR protein” may be used interchangeably with “CRISPR-Cas protein”, irrespective of whether the CRISPR protein has altered, such as increased or decreased (or no) enzymatic activity, compared to the wild type CRISPR protein.

Computational analysis of the primary structure of Cas7-11 nucleases reveals 5 distinct domain regions.

Based on the above information, mutants can be generated which lead to inactivation of the enzyme or which modify the double strand nuclease to nickase activity. In alternative embodiments, this information is used to develop enzymes with reduced off-target effects.

In certain of the above-described Cas7-11 enzymes, the enzyme is modified by mutation of one or more residues (in the Cas7-like domains as well as the small subunit).

Orthologs of Cas7-11

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related or are only partially structurally related. An “orthologue” of a protein as used herein is a protein of a different species which performs the same or a similar function as the protein it is an orthologue of. Orthologous proteins may but need not be structurally related or are only partially structurally related. Homologs and orthologs may be identified by homology modelling (see, e.g., Greer, Science vol. 228 (1985) 1055, and Blundell et al. Eur J Biochem vol 172(1988), 513) or “structural BLAST” (Dey F, Cliff Zhang Q, Petrey D, Honig B. Toward a “structural BLAST”: using structural relationships to infer function. Protein Sci. 2013 April; 22(4):359-66. Doi: 10.1002/pro.2225.). See also Shmakov et al. (2015) for application in the field of CRISPR-Cas loci.

The present disclosure encompasses the use of a Cas7-11 effector protein, derived from a Cas7-11 locus denoted as subtype III-E. Herein such effector proteins are also referred to as “Cas7-11p”, e.g., a Cas7-11 protein (and such effector protein or Cas7-11 protein or protein derived from a Cas7-11 locus is also called “CRISPR-Cas protein”).

In particular embodiments, the effector protein is a Cas7-11 effector protein from an organism from a genus comprising Candidatus Jettenia caeni, Candidatus Scalindua brodae, Desulfobacteraceae, Candidatus Magnetomorum, Desulfonema Ishimotonii, Candidatus Brocadia, Deltaproteobacteria, Syntrophorhabdaceae, or Nitrospirae.

Delivery Cas7-11 Effector

In some embodiments, the Cas7-11 effector and/or peptide sequence are introduced into a cell as a nucleic acid encoding each protein. The nucleic acid introduced into the eukaryotic cell is a plasmid DNA or viral vector. In some embodiments, the Cas7-11 effector and/or peptide sequence are introduced into a cell via a ribonucleoprotein (RNP).

Preferably, delivery is in the form of a vector which may be a viral vector, such as a lenti- or baculo- or adeno-viral/adeno-associated viral vectors, but other means of delivery are known (such as yeast systems, microvesicles, gene guns/means of attaching vectors to gold nanoparticles) and are provided. The viral vector may be selected from a variety of families/genera of viruses, including, but not limited to Myoviridae, Siphoviridae, Podoviridae, Corticoviridae, Lipothrixviridae, Poxviridae, Iridoviridae, Adenoviridae, Polyomaviridae, Papillomaviridae, Mimiviridae, Pandoravirusa, Salterprovirusa, Inoviridae, Microviridae, Parvoviridae, Circoviridae, Hepadnaviridae, Caulimoviridae, Retroviridae, Cystoviridae, Reoviridae, Bimaviridae, Totiviridae, Partitiviridae, Filoviridae, Orthomyxoviridae, Deltavirusa, Leviviridae, Picomaviridae, Mamaviridae, Secoviridae, Potyviridae, Caliciviridae, Hepeviridae, Astroviridae, Nodaviridae, Tetraviridae, Luteoviridae, Tombusviridae, Coronaviridae, Arteriviridae, Flaviviridae, Togaviridae, Virgaviridae, Bromoviridae, Tymoviridae, Alphaflexiviridae, Sobemovirusa, Idaeovirusa, and Herpesviridae.

A vector may mean not only a viral or yeast system (for instance, where the nucleic acids of interest may be operably linked to and under the control of (in terms of expression, such as to ultimately provide a processed RNA) a promoter), but also direct delivery of nucleic acids into a host cell. For example, baculoviruses may be used for expression in insect cells. These insect cells may, in turn be useful for producing large quantities of further vectors, such as AAV or lentivirus adapted for delivery of the present disclosure. Also envisaged is a method of delivering the Cas7-11 effector and/or peptide sequence comprising delivering to a cell mRNAs encoding each.

In some embodiments, expression of a nucleic acid sequence encoding the Cas7-11 effector and/or peptide sequence may be driven by a promoter. In some embodiments, a single promoter drives expression of a nucleic acid sequence encoding the Cas7-11 effector. In some embodiments, the Cas7-11 effector and guide sequence(s) are operably linked to and expressed from the same promoter. In some embodiments, the Cas7-11 and guide sequence(s) are expressed from different promoters. For example, the promoter(s) can be, but are not limited to, a UBC promoter, a PGK promoter, an EF1A promoter, a CMV promoter, an EFS promoter, a SV40 promoter, and a TRE promoter. The promoter may be a weak or a strong promoter. The promoter may be a constitutive promoter or an inducible promoter. In some embodiments, the promoter can also be an AAV ITR, and can be advantageous for eliminating the need for an additional promoter element, which can take up space in the vector. The additional space freed up by use of an AAV ITR can be used to drive the expression of additional elements, such as guide sequences. In some embodiments, the promoter may be a tissue specific promoter.

In some embodiments, an enzyme coding sequence encoding Cas7-11 effector and/or peptide sequence is codon-optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a Cas7-11 effector correspond to the most frequently used codon for a particular amino acid.

In some embodiments, a vector encodes a Cas7-11 effector and/or peptide sequence comprising one or more nuclear localization sequences (NLSs), such as about or more than about 1, 2, 3, 4, 5, 6, 7, 8, 9, 10, or more NLSs. In some embodiments, the Cas7-11 protein comprises about or more than 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Typically, an NLS consists of one or more short sequences of positively charged lysines or arginines exposed on the protein surface, bur other types of NLS are known. In some embodiments, the NLS is between two domains, for example between the Cas7-11 effector protein and the viral protein. The NLS may also be between two functional domains separated or flanked by a glycine-serine linker.

In general, the one or more NLSs are of sufficient strength to drive accumulation of the Cas7-11 effector and/or peptide sequence in a detectable amount in the nucleus of a eukaryotic cell. In general, strength of nuclear localization activity may derive from the number of NLSs in the Cas7-11 effector and/or other peptide sequences, the particular NLS used, or a combination of these factors. Detection of accumulation in the nucleus may be performed by any suitable technique. For example, a detectable marker may be fused to the Cas7-11 effector and/or peptide sequence, such that location within a cell may be visualized, such as in combination with a means for detecting the location of the nucleus (e.g., a stain specific for the nucleus such as DAPI). Examples of detectable markers include fluorescent proteins (such as green fluorescent proteins, or GFP; RFP; CFP), and epitope tags (HA tag, FLAG tag, SNAP tag). Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some aspects, the disclosure provides methods comprising delivering one or more polynucleotides, such as one or more vectors as described herein, one or more transcripts thereof, and/or one or proteins transcribed therefrom, to a host cell. In some aspects, the disclosure further provides cells produced by such methods, and organisms (such as animals, plants, or fungi) comprising or produced from such cells. In some embodiments, a Cas protein in combination with (and optionally complexed) with a guide sequence is delivered to a cell. Conventional viral and non-viral based gene transfer methods can be used to introduce nucleic acids in mammalian cells or target tissues. Such methods can be used to administer nucleic acids encoding a Cas7-11 effector and/or a polypeptide to cells in culture, or in a host organism. Non-viral vector delivery systems include DNA plasmids, RNA (e.g., a transcript of a vector described herein), naked nucleic acid, nucleic acid complexed with a delivery vehicle, such as a liposome, and ribonucleoprotein. Viral vector delivery systems include DNA and RNA viruses, which have either episomal or integrated genomes after delivery to the cell. For a review of gene therapy procedures, see Anderson, Science 256:808-8313 (1992); Navel and Felgner, TIBTECH 11:211-217 (1993); Mitani and Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer and Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994), which are incorporated herein by reference in their entirety.

The Cas7-11 effector and/or peptide sequence can be delivered using adeno-associated virus (AAV), lentivirus, adenovirus, or other viral vector types, or combinations thereof. In some embodiments, one or more Cas7-11 effectors and/or one or more guide RNAs can be packaged into one or more viral vectors. In some embodiments, the Cas7-11 effector and/or peptide sequence can be delivered via AAV as a trans-splicing system, similar to Lai et al. (Nature Biotechnology, 2005, DOI: 10.1038/nbt1153). In some embodiments, the viral vector is delivered to the tissue of interest by, for example, an intramuscular injection, while other times the viral delivery is via intravenous, transdermal, intranasal, oral, mucosal, intrathecal, intracranial, or other delivery methods. Such delivery may be either via a single dose, or multiple doses. One skilled in the art understands that the actual dosage to be delivered herein may vary greatly depending upon a variety of factors, such as the vector chosen, the target cell, organism, or tissue, the general condition of the subject to be treated, the degree of transformation/modification sought, the administration route, the administration mode, the type of transformation/modification sought, etc.

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo), or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In certain embodiments, delivery of the Cas7-11 and/or peptide sequence to a cell is non-viral. In certain embodiments, the non-viral delivery system is selected from a ribonucleoprotein, cationic lipid vehicle, electroporation, nucleofection, calcium phosphate transfection, transfection through membrane disruption using mechanical shear forces, mechanical transfection, and nanoparticle delivery.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. Cell lines are available from a variety of sources known to those with skill in the art (see, e.g., the American Type Culture Collection (ATCC) (Manassas, VA). In some embodiments, a cell transfected with one or more vectors described herein is used to establish a new cell line comprising one or more vector-derived sequences.

Guide Molecules

The system may comprise a guide molecule. The guide molecule may comprise a guide sequence. In certain cases, the guide sequence may be linked to a direct repeat sequence. In some cases, the system may comprise a nucleotide sequence encoding the guide molecule. The guide molecule may form a complex with the dead Cas7-11 protein and directs the complex to bind the target RNA sequence at one or more codons encoding an amino acid that is post-translationally modified. The guide sequence may be capable of hybridizing with a target RNA sequence comprising an Adenine or Cytidine encoding said amino acid to form an RNA duplex, wherein said guide sequence comprises a non-pairing nucleotide at a position corresponding to said Adenine or Cytidine resulting in a mismatch in the RNA duplex formed. The guide sequence may comprise one or more mismatch corresponding to different adenosine sites in the target sequence. In certain cases, guide sequence may comprise multiple mismatches corresponding to different adenosine sites in the target sequence. In cases where two guide molecules are used, the guide sequence of each of the guide molecules may comprise a mismatch corresponding to a different adenosine site in the target sequence.

In general, a CRISPR system is characterized by elements that promote the formation of a CRISPR complex at the site of a target sequence. In the context of formation of a CRISPR complex, “target sequence” refers to a sequence to which a guide sequence is designed to have complementarity, where hybridization between a target DNA sequence and a guide sequence promotes the formation of a CRISPR complex.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. The precise sequence and length requirements for the PAM differ depending on the Cas7-11 protein used, but PAMs are typically 2-8 base pair sequences adjacent the protospacer (that is, the target sequence). Examples of the natural PAM sequences for different Cas7-11 orthologues are provided herein below and the skilled person will be able to identify further PAM sequences for use with a given Cas7-11 protein. In certain embodiments, the Cas7-11 protein has been modified to recognize a non-natural PAM, such as recognizing a PAM having a sequence or comprising a sequence YCN, YCV, AYV, TYV, RYN, RCN, TGYV(SEQ ID NO:79), NTTN(SEQ ID NO:80), TN, TRTN(SEQ ID NO:81), TYTV(SEQ ID NO:82), TYCT(SEQ ID NO:83), TYCN(SEQ ID NO:84), TRTN(SEQ ID NO:81), NTTN(SEQ ID NO:80), TACT(SEQ ID NO:85), TYCC(SEQ ID NO:86), TRTC(SEQ ID NO:87), TATV(SEQ ID NO:88), NTTV(SEQ ID NO:89), TTV, TSTG(SEQ ID NO:90), TVTS(SEQ ID NO:91), TYYS(SEQ ID NO:92), TCYS(SEQ ID NO:93), TBYS(SEQ ID NO:94), TCYS(SEQ ID NO:93), TNYS(SEQ ID NO:95), TYYS(SEQ ID NO:92), TNTN(SEQ ID NO:96), TSTG(SEQ ID NO:90), TTCC(SEQ ID NO:97), TCCC(SEQ ID NO:98), TATC(SEQ ID NO:99), TGTG(SEQ ID NO:100), TCTG(SEQ ID NO:101), TYCV(SEQ ID NO:102), or TCTC(SEQ ID NO:103).

The terms “guide molecule” and “guide RNA” are used interchangeably herein to refer to RNA-based molecules that are capable of forming a complex with a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA specifically encompasses RNA-based molecules having one or more chemically modifications (e.g., by chemical linking two ribonucleotides or by replacement of one or more ribonucleotides with one or more deoxyribonucleotides), as described herein.

As used herein, the term “guide sequence” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In the context of the present disclosure the target nucleic acid sequence or target sequence is the sequence comprising the target adenosine to be deaminated also referred to herein as the “target adenosine”. In some embodiments, except for the intended dA-C mismatch, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, ClustalX, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In some embodiments, the guide molecule comprises a guide sequence that is designed to have at least one mismatch with the target sequence, such that an RNA duplex formed between the guide sequence and the target sequence comprises a non-pairing C in the guide sequence opposite to the target A for deamination on the target sequence. In some embodiments, aside from this A-C mismatch, the degree of complementarity, when optimally aligned using a suitable alignment algorithm, is about or more than about 50%, 60%, 75%, 80%, 85%, 90%, 95%, 97.5%, 99%, or more. In some cases, the distance between the non-pairing C and the 5′ end of the guide sequence is from about 10 to about 50, e.g., from about 10 to about 20, from about 15 to about 25, from about 20 to about 30, from about 25 to about 35, from about 30 to about 40, from about 35 to about 45, or from about 40 to about 50 nucleotides (nt) in length. In certain example. In some cases, the distance between the non-pairing C and the 3′ end of the guide sequence is from about 10 to about 50, e.g., from about 10 to about 20, from about 15 to about 25, from about 20 to about 30, from about 25 to about 35, from about 30 to about 40, from about 35 to about 45, or from about 40 to about 50 nucleotides (nt) in length. In one example, the distance between the non-pairing C and the 5′ end of said guide sequence is from about 20 to about 30 nucleotides.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence has a length from about 10 to about 100, e.g., from about 20 to about 60, from about 20 to about 55, from about 20 to about 53, from about 25 to about 53, from about 29 to about 53, from about 20 to about 30, from about 25 to about 35, from about 30 to about 40, from about 35 to about 45, from about 40 to about 50, from about 45 to about 55, from about 50 to about 60, from about 55 to about 65, from about 60 to about 70, from about 70 to about 80, from about 80 to about 90, or from about 90 to about 100 nucleotides (nt) long that is capable of forming an RNA duplex with a target sequence. In certain example, the guide sequence has a length from about 20 to about 53 nt capable of forming said RNA duplex with said target sequence. In certain example, the guide sequence has a length from about 25 to about 53 nt capable of forming said RNA duplex with said target sequence. In certain example, the guide sequence has a length from about 29 to about 53 nt capable of forming said RNA duplex with said target sequence. In certain example, the guide sequence has a length from about 40 to about 50 nt capable of forming said RNA duplex with said target sequence. In some examples, the guide sequence comprises a non-pairing Cytosine at a position corresponding to said Adenine resulting in an A-C mismatch in the RNA duplex formed. The guide sequence is selected so as to ensure that it hybridizes to the target sequence comprising the adenosine to be deaminated.

In some embodiments, the guide sequence is about 10 nt to about 100 nt long and hybridizes to the target DNA strand to form an almost perfectly matched duplex, except for having a dA-C mismatch at the target adenosine site. Particularly, in some embodiments, the dA-C mismatch is located close to the center of the target sequence (and thus the center of the duplex upon hybridization of the guide sequence to the target sequence), thereby restricting the nucleotide deaminase to a narrow editing window (e.g., about 4 bp wide). In some embodiments, the target sequence may comprise more than one target adenosine to be deaminated. In further embodiments, the target sequence may further comprise one or more dA-C mismatch 3′ to the target adenosine site. In some embodiments, to avoid off-target editing at an unintended Adenine site in the target sequence, the guide sequence can be designed to comprise a non-pairing Guanine at a position corresponding to said unintended Adenine to introduce a dA-G mismatch, which is catalytically unfavorable for certain nucleotide deaminases such as ADAR1 and ADAR2. See Wong et al., RNA 7:846-858 (2001), which is incorporated herein by reference in its entirety.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%), 1%), or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, it is of interest to reduce the susceptibility of the guide molecule to RNA cleavage, such as to cleavage by Cas7-11. Accordingly, in particular embodiments, the guide molecule is adjusted to avoid cleavage by Cas7-11 or other RNA-cleaving enzymes.

In some embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The disclosure accordingly comprehends a guide molecule that responds to normal or pathological physiological conditions, including without limitation pH, hypoxia, O₂ concentration, temperature, protein concentration, enzymatic concentration, lipid structure, light exposure, mechanical disruption (e.g., ultrasound waves), magnetic fields, electric fields, or electromagnetic radiation.

Adenosine Deaminase

The system may further comprise an adenosine deaminase or catalytic domain thereof. The adenosine deaminase protein or catalytic domain thereof deaminates an Adenine or Cytidine at the one or more codons thereby changing the codon to encode for an amino acid that is not post-translationally modified. The term “adenosine deaminase” or “adenosine deaminase protein” as used herein refers to a protein, a polypeptide, or one or more functional domain(s) of a protein or a polypeptide that is capable of catalyzing a hydrolytic deamination reaction that converts an adenine (or an adenine moiety of a molecule) to a hypoxanthine (or a hypoxanthine moiety of a molecule), as shown below. In some embodiments, the adenine-containing molecule is an adenosine (A), and the hypoxanthine-containing molecule is an inosine (I). The adenine-containing molecule can be deoxyribonucleic acid (DNA) or ribonucleic acid (RNA).

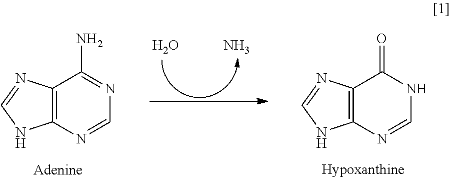

According to the present disclosure, adenosine deaminases that can be used in connection with the present disclosure include, but are not limited to, members of the enzyme family known as adenosine deaminases that act on RNA (ADARs), members of the enzyme family known as adenosine deaminases that act on tRNA (ADATs), and other adenosine deaminase domain-containing (AD AD) family members. According to the present disclosure, the adenosine deaminase is capable of targeting adenine in an RNA/DNA and RNA duplexes. Indeed, Zheng et al. (Nucleic Acids Res. 2017, 45(6): 3369-3377) demonstrate that ADARs can carry out adenosine to inosine editing reactions on RNA/DNA and RNA/RNA duplexes. The adenosine deaminase can be modified to increase its ability to edit DNA in an RNA/DNA RNA duplex.

In some embodiments, the adenosine deaminase is derived from one or more metazoa species, including but not limited to, mammals, birds, frogs, squids, fish, flies, and worms. In some embodiments, the adenosine deaminase is a human, cephalopod (e.g., squid) or Drosophila adenosine deaminase. In certain examples, the adenosine deaminase is a human adenosine deaminase. In certain examples, the adenosine deaminase is a cephalopod adenosine deaminase. In certain examples, the adenosine deaminase is a Drosophila adenosine deaminase.

Cytidine Deaminase

The term “cytidine deaminase” or “cytidine deaminase protein” as used herein refers to a protein, a polypeptide, or one or more functional domain(s) of a protein or a polypeptide that is capable of catalyzing a hydrolytic deamination reaction that converts a cytosine (or a cytosine moiety of a molecule) to an uracil (or an uracil moiety of a molecule), as shown below. In some embodiments, the cytosine-containing molecule is a cytidine (C), and the uracil-containing molecule is a uridine (U). The cytosine-containing molecule can be deoxyribonucleic acid (DNA) or ribonucleic acid (RNA).

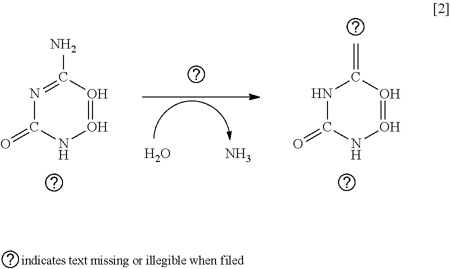

According to the present disclosure, cytidine deaminases that can be used in connection with the present disclosure include, but are not limited to, members of the enzyme family known as apolipoprotein B mRNA-editing complex (APOBEC) family deaminase, an activation-induced deaminase (AID), or a cytidine deaminase 1 (CDA1). In particular embodiments, the deaminase in an APOBEC 1 deaminase, an APOBEC2 deaminase, an APOBEC3A deaminase, an APOBEC3B deaminase, an APOBEC3C deaminase, and APOBEC3D deaminase, an APOBEC3E deaminase, an APOBEC3F deaminase an APOBEC3G deaminase, an APOBEC3H deaminase, or an APOBEC4 deaminase. The cytidine deaminase can be modified to increase its ability to edit DNA in an RNA/DNAn RNA duplex.

In some embodiments, the cytidine deaminase is derived from one or more metazoa species, including but not limited to, mammals, birds, frogs, squids, fish, flies, and worms. In some embodiments, the cytidine deaminase is a human, primate, cow, dog, rat, or mouse cytidine deaminase.

CD (cytidine deaminase)-functionalized CRISPR system for RNA editing can be used for C to U conversions. In some embodiments, the cytidine deaminase protein or catalytic domain thereof is a human, rat or lamprey cytidine deaminase protein or catalytic domain thereof. In some embodiments, the cytidine deaminase protein or catalytic domain thereof is an apolipoprotein B mRNA-editing complex (APOBEC) family deaminase, an activation-induced deaminase (AID), or a cytidine deaminase 1 (CDA1). In some embodiments, the cytidine deaminase protein or catalytic domain thereof is an APOBEC1 deaminase comprising one or more mutations corresponding to W90A, W90Y, R118A, H121R, H122R, R126A, R126E, or R132E in rat APOBEC1, or an APOBEC3G deaminase comprising one or more mutations corresponding to W285A, W285Y, R313A, D316R, D317R, R320A, R320E, or R326E in human APOBEC3G. In some embodiments, the cytidine deaminase protein or catalytic domain thereof is delivered together with an uracil glycosylase inhibitor (UGI), where said UGI is covalently linked to said cytidine deaminase protein or catalytic domain thereof and/or said catalytically inactive Cas7-11 protein.

Cas7-11-APOBEC fusions can perform C-to-U editing of RNA. APOBEC substrates are ssRNA and the Cas7-11-APOBEC can therefore target regions of the RNA around the guide/target duplex. Cas7-11-APOBEC fusions can perform C to U knockdown via stop codon introduction. In addition to correcting pathogenic U to C mutations that arise during the cellular life cycle, Cas7-11-APOBEC fusions can lead to the introduction of stop codons by converting a CAA, CGA, or CAG to TAA, TGA, or TAG, respectively. APOBEC orthologs in fusion with Cas7-11 can increase the efficiency of C-to-U editing or can allow for additional types of base conversions. Mutating the APOBEC from the Cas7-11-APOBEC can lead to fusions with specific dsRNA activity, base flip activity and increased activity.

Ortholog Truncations

Table 1 below shows examples of truncations of Cas7-11 orthologs.

TABLE 1
Residues replaced with GGGS

| | | Start | | End | | |
|---|---|---|---|---|---|---|
| | AA of WT | AA | position | AA | position | AA of Cas7-11S |
| DiCas7-11S | 1601 | Met | 979 | Ser | 1293 | 1291 |
| SbCas7-11S | 1722 | Met | 1032 | Ser | 1387 | 1371 |
| HvsCas7-11S | 1528 | Met | 955 | Ser | 1240 | 1247 |
| CjcCas7-11S | 1812 | Leu | 1109 | Ser | 1406 | 1519 |

Correspond to amino acid ranges predicted for truncations that can make the orthologs smaller yet still functional.
The truncated orthologs can be useful for delivery applications, like AAV, that have packaging size limits.
“AA of WT” denotes the length of the unmodified WT ortholog. “AA of Cas7-11S” denotes the length of the ortholog after the proposed deletion between the “Start” and “End” positions and insertion of a GGGS linker. The GGGS linker helps maintain flexibility of the fused protein domains.

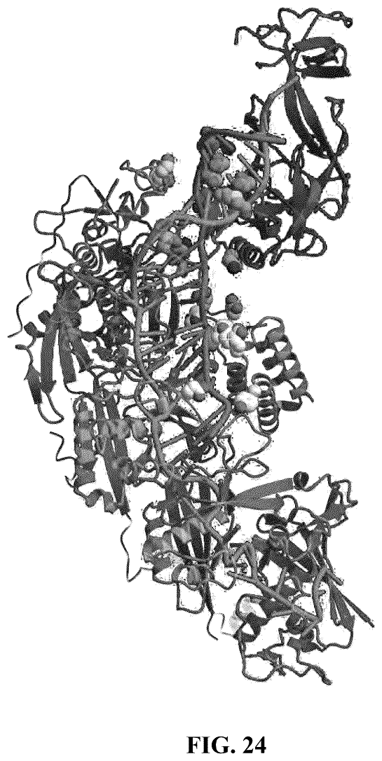

Based on conservation and structural homology to the DiCas7-11 structure, predictions can be made in the four orthologs shown in FIG. 24 and in Table 1 above for non-functional regions to truncate out, where the orthologs retain catalytic RNA cleavage activity. The column labelled START signifies the amino acid at the beginning of the proposed truncation and the column labelled END signifies the amino acid at the end of the truncation. By subtracting those two numbers, the length of the truncation can be determined. The column labelled AA of WT for each ortholog refers to the natural length of the ortholog in amino acids. For example, the first entry, DiCas7-11, refers to the non-truncated form, which is 1601 amino acids. After the indicated truncation from position 979 to 1293 and the addition of the GGGS(SEQ ID NO:77) linker, the modified protein will be 1291 amino acids in length, as indicated in the column AA of Cas7-11S.

The GGGS(SEQ ID NO:77) linker is used to replace these regions because it is a small, flexible linker that ensures that the truncation is functional (i.e., can bind a crRNA and target RNA for cleavage).

The resulting truncated orthologs are easier to package for delivery to cells because they are 285-355 amino acids shorter in length and are still predicted to retain RNA knockdown and RNA binding function based on the DiCas7-11S truncation.

In some embodiments, the truncation could be made without inserting the GGGS(SEQ ID NO:77) linker. In addition, other linkers could be used or be placed in other domains. Other possible linker sequences, include, but are not limited to:

GS
(SEQ ID NO: 104)
GSGGGGS
(SEQ ID NO: 105)
GGGGSGGGGSGGGGS
(SEQ ID NO: 106)
EAAAK
(SEQ ID NO: 107)
EAAAKEAAAKEAAAK
(SEQ ID NO: 108)
GGSGGSGGSGGSGGSGGS
(SEQ ID NO: 109)
SGSETPGTSESATPES

Residue Mutations

Amino acid residues that are located near or cRNA or the target RNA can be varied also. Table 2 shows examples of amino acid residue mutations to boost the activity of DiCas7-11. The column labeled AA shows the identity of the residue and the position column indicates the position of the amino acid in DiCas7-11. In some embodiments, one or more individual or multiple residues can be mutated to another amino acid residue or multiple residues. In some embodiments, the individual amino acid is mutated to an arginine or a lysine.

TABLE 2
Mutations to Arg or Lys might increase the target
RNA binding and cleavage seems more feasible

| DiCas7-11 | AA | position |
|---|---|---|
| DiCas7-11 | Glu | 280 |
| DiCas7-11 | Tyr | 281 |
| DiCas7-11 | His | 306 |
| DiCas7-11 | Asp | 307 |
| DiCas7-11 | Asp | 311 |
| DiCas7-11 | Tyr | 313 |
| DiCas7-11 | Tyr | 360 |
| DiCas7-11 | Glu | 513 |
| DiCas7-11 | Asp | 980 |
| DiCas7-11 | Asp | 982 |
| DiCas7-11 | Asp | 989 |
| DiCas7-11 | Asp | 1164 |
| DiCas7-11 | Asn | 1247 |
| DiCas7-11 | Glu | 1392 |
| DiCas7-11 | Asn | 1441 |
| DiCas7-11 | Asp | 1568 |
| DiCas7-11 | Asp | 1581 |

Listed are residues that can modulate activity and/or RNA binding.
The residues are highlighted in the structure model shown in FIG. 24.

### EXAMPLES

While several experimental Examples are contemplated, these Examples are intended to be non-limiting.

### Materials

Table 3 below shows examples of protein amino acid sequences (N-C terminal).

TABLE 3

| SEQ ID | |
|---|---|
| NO/ | |
| Description | Sequence |
| SEQ ID | MTTTMKISIEFLEPFRMTKWQESTRRNKNNKEFVRGQAFARWHRNKK |
| NO: 1 | DNTKGRPYITGTLLRSAVIRSAENLLTLSDGKISEKTCCPGKFDTEDKD |
| huDiCas7- | RLLQLRQRSTLRWTDKNPCPDNAETYCPFCELLGRSGNDGKKAEKKD |
| 11 | WRFRIHFGNLSLPGKPDFDGPKAIGSQRVLNRVDFKSGKAHDFFKAYE |
| | VDHTRFPRFEGEITIDNKVSAEARKLLCDSLKFTDRLCGALCVIRFDEY |
| | TPAADSGKQTENVQAEPNANLAEKTAEQIISILDDNKKTEYTRLLADAI |
| | RSLRRSSKLVAGLPKDHDGKDDHYLWDIGKKKKDENSVTIRQILTTSA |
| | DTKELKNAGKWREFCEKLGEALYLKSKDMSGGLKITRRILGDAEFHG |
| | KPDRLEKSRSVSIGSVLKETVVCGELVAKTPFFFGAIDEDAKQTDLQVL |
| | LTPDNKYRLPRSAVRGILRRDLQTYFDSPCNAELGGRPCMCKTCRIMR |
| | GITVMDARSEYNAPPEIRHRTRINPFTGTVAEGALFNMEVAPEGIVFPF |
| | QLRYRGSEDGLPDALKTVLKWWAEGQAFMSGAASTGKGRFRMENA |
| | KYETLDLSDENQRNDYLKNWGWRDEKGLEELKKRLNSGLPEPGNYR |
| | DPKWHEINVSIEMASPFINGDPIRAAVDKRGTDVVTFVKYKAEGEEAK |
| | PVCAYKAESFRGVIRSAVARIHMEDGVPLTELTHSDCECLLCQIFGSEY |
| | EAGKIRFEDLVFESDPEPVTFDHVAIDRFTGGAADKKKFDDSPLPGSPA |
| | RPLMLKGSFWIRRDVLEDEEYCKALGKALADVNNGLYPLGGKSAIGY |
| | GQVKSLGIKGDDKRISRLMNPAFDETDVAVPEKPKTDAEVRIEAEKVY |
| | YPHYFVEPHKKVEREEKPCGHQKFHEGRLTGKIRCKLITKTPLIVPDTS |
| | NDDFFRPADKEARKEKDEYHKSYAFFRLHKQIMIPGSELRGMVSSVYE |
| | TVTNSCFRIFDETKRLSWRMDADHQNVLQDFLPGRVTADGKHIQKFSE |
| | TARVPFYDKTQKHFDILDEQEIAGEKPVRMWVKRFIKRLSLVDPAKHP |
| | QKKQDNKWKRRKEGIATFIEQKNGSYYFNVVTNNGCTSFHLWHKPD |
| | NFDQEKLEGIQNGEKLDCWVRDSRYQKAFQEIPENDPDGWECKEGYL |
| | HVVGPSKVEFSDKKGDVINNFQGTLPSVPNDWKTIRTNDFKNRKRKN |
| | EPVFCCEDDKGNYYTMAKYCETFFFDLKENEEYEIPEKARIKYKELLR |
| | VYNNNPQAVPESVFQSRVARENVEKLKSGDLVYFKHNEKYVEDIVPV |
| | RISRTVDDRMIGKRMSADLRPCHGDWVEDGDLSALNAYPEKRLLLRH |
| | PKGLCPACRLFGTGSYKGRVRFGFASLENDPEWLIPGKNPGDPFHGGP |
| | VMLSLLERPRPTWSIPGSDNKFKVPGRKFYVHHHAWKTIKDGNHPTT |
| | GKAIEQSPNNRTVEALAGGNSFSFEIAFENLKEWELGLLIHSLQLEKGL |
| | AHKLGMAKSMGFGSVEIDVESVRLRKDWKQWRNGNSEIPNWLGKGF |
| | AKLKEWFRDELDFIENLKKLLWFPEGDQAPRVCYPMLRKKDDPNGNS |
| | GYEELKDGEFKKEDRQKKLTTPWTPWA |
| SEQ ID | MTTTMKISIEFLEPFRMTKWQESTRRNKNNKEFVRGQAFARWHRNKK |
| NO: 2 | DNTKGRPYITGTLLRSAVIRSAENLLTLSDGKISEKTCCPGKFDTEDKD |
| huDiCas7- | RLLQLRQRSTLRWTDKNPCPDNAETYCPFCELLGRSGNDGKKAEKKD |
| 11- | WRFRIHFGNLSLPGKPDFDGPKAIGSQRVLNRVDFKSGKAHDFFKAYE |
| R978- | VDHTRFPRFEGEITIDNKVSAEARKLLCDSLKFTDRLCGALCVIRFDEY |
| GGGS- | TPAADSGKQTENVQAEPNANLAEKTAEQIISILDDNKKTEYTRLLADAI |
| R1294 | RSLRRSSKLVAGLPKDHDGKDDHYLWDIGKKKKDENSVTIRQILTTSA |
| | DTKELKNAGKWREFCEKLGEALYLKSKDMSGGLKITRRILGDAEFHG |
| | KPDRLEKSRSVSIGSVLKETVVCGELVAKTPFFFGAIDEDAKQTDLQVL |
| | LTPDNKYRLPRSAVRGILRRDLQTYFDSPCNAELGGRPCMCKTCRIMR |
| | GITVMDARSEYNAPPEIRHRTRINPFTGTVAEGALFNMEVAPEGIVFPF |
| | QLRYRGSEDGLPDALKTVLKWWAEGQAFMSGAASTGKGRFRMENA |
| | KYETLDLSDENQRNDYLKNWGWRDEKGLEELKKRLNSGLPEPGNYR |
| | DPKWHEINVSIEMASPFINGDPIRAAVDKRGTDVVTFVKYKAEGEEAK |
| | PVCAYKAESFRGVIRSAVARIHMEDGVPLTELTHSDCECLLCQIFGSEY |
| | EAGKIRFEDLVFESDPEPVTFDHVAIDRFTGGAADKKKFDDSPLPGSPA |
| | RPLMLKGSFWIRRDVLEDEEYCKALGKALADVNNGLYPLGGKSAIGY |
| | GQVKSLGIKGDDKRISRLMNPAFDETDVAVPEKPKTDAEVRIEAEKVY |
| | YPHYFVEPHKKVEREEKPCGHQKFHEGRLTGKIRCKLITKTPLIVPDTS |
| | NDDFFRPADKEARKEKDEYHKSYAFFRLHKQIMIPGSELRGMVSSVYE |
| | TVTNSCFRIFDETKRLSWRGGGSRTVDDRMIGKRMSADLRPCHGDWV |
| | EDGDLSALNAYPEKRLLLRHPKGLCPACRLFGTGSYKGRVRFGFASLE |
| | NDPEWLIPGKNPGDPFHGGPVMLSLLERPRPTWSIPGSDNKFKVPGRK |
| | FYVHHHAWKTIKDGNHPTTGKAIEQSPNNRTVEALAGGNSFSFEIAFE |
| | NLKEWELGLLIHSLQLEKGLAHKLGMAKSMGFGSVEIDVESVRLRKD |
| | WKQWRNGNSEIPNWLGKGFAKLKEWFRDELDFIENLKKLLWFPEGD |
| | QAPRVCYPMLRKKDDPNGNSGYEELKDGEFKKEDRQKKLTTPWTPW |
| | A |
| SEQ ID | MTTTMKISIEFLEPFRMTKWQESTRRNKNNKEFVRGQAFARWHRNKK |
| NO: 3 | DNTKGRPYITGTLLRSAVIRSAENLLTLSDGKISEKTCCPGKFDTEDKD |
| huDiCas7- | RLLQLRQRSTLRWTDKNPCPDNAETYCPFCELLGRSGNDGKKAEKKD |
| 11- | WRFRIHFGNLSLPGKPDFDGPKAIGSQRVLNRVDFKSGKAHDFFKAYE |
| S1006- | VDHTRFPRFEGEITIDNKVSAEARKLLCDSLKFTDRLCGALCVIRFDEY |
| GGGS- | TPAADSGKQTENVQAEPNANLAEKTAEQIISILDDNKKTEYTRLLADAI |
| D1221 | RSLRRSSKLVAGLPKDHDGKDDHYLWDIGKKKKDENSVTIRQILTTSA |
| | DTKELKNAGKWREFCEKLGEALYLKSKDMSGGLKITRRILGDAEFHG |
| | KPDRLEKSRSVSIGSVLKETVVCGELVAKTPFFFGAIDEDAKQTDLQVL |
| | LTPDNKYRLPRSAVRGILRRDLQTYFDSPCNAELGGRPCMCKTCRIMR |
| | GITVMDARSEYNAPPEIRHRTRINPFTGTVAEGALFNMEVAPEGIVFPF |
| | QLRYRGSEDGLPDALKTVLKWWAEGQAFMSGAASTGKGRFRMENA |
| | KYETLDLSDENQRNDYLKNWGWRDEKGLEELKKRLNSGLPEPGNYR |
| | DPKWHEINVSIEMASPFINGDPIRAAVDKRGTDVVTFVKYKAEGEEAK |
| | PVCAYKAESFRGVIRSAVARIHMEDGVPLTELTHSDCECLLCQIFGSEY |
| | EAGKIRFEDLVFESDPEPVTFDHVAIDRFTGGAADKKKFDDSPLPGSPA |
| | RPLMLKGSFWIRRDVLEDEEYCKALGKALADVNNGLYPLGGKSAIGY |
| | GQVKSLGIKGDDKRISRLMNPAFDETDVAVPEKPKTDAEVRIEAEKVY |
| | YPHYFVEPHKKVEREEKPCGHQKFHEGRLTGKIRCKLITKTPLIVPDTS |
| | NDDFFRPADKEARKEKDEYHKSYAFFRLHKQIMIPGSELRGMVSSVYE |
| | TVTNSCFRIFDETKRLSWRMDADHQNVLQDFLPGRVTADGKHIQKFS |
| | GGGSDLKENEEYEIPEKARIKYKELLRVYNNNPQAVPESVFQSRVARE |
| | NVEKLKSGDLVYFKHNEKYVEDIVPVRISRTVDDRMIGKRMSADLRPC |
| | HGDWVEDGDLSALNAYPEKRLLLRHPKGLCPACRLFGTGSYKGRVRF |
| | GFASLENDPEWLIPGKNPGDPFHGGPVMLSLLERPRPTWSIPGSDNKFK |
| | VPGRKFYVHHHAWKTIKDGNHPTTGKAIEQSPNNRTVEALAGGNSFSF |
| | EIAFENLKEWELGLLIHSLQLEKGLAHKLGMAKSMGFGSVEIDVESVR |
| | LRKDWKQWRNGNSEIPNWLGKGFAKLKEWFRDELDFIENLKKLLWFP |
| | EGDQAPRVCYPMLRKKDDPNGNSGYEELKDGEFKKEDRQKKLTTPW |
| | TPWA |
| SEQ ID | MTTTMKISIEFLEPFRMTKWQESTRRNKNNKEFVRGQAFARWHRNKK |
| NO: 4 | DNTKGRPYITGTLLRSAVIRSAENLLTLSDGKISEKTCCPGKFDTEDKD |
| huDiCas7- | RLLQLRQRSTLRWTDKNPCPDNAETYCPFCELLGRSGNDGKKAEKKD |
| 11- | WRFRIHFGNLSLPGKPDFDGPKAIGSQRVLNRVDFKSGKAHDFFKAYE |
| R1045- | VDHTRFPRFEGEITIDNKVSAEARKLLCDSLKFTDRLCGALCVIRFDEY |
| GGGS- | TPAADSGKQTENVQAEPNANLAEKTAEQIISILDDNKKTEYTRLLADAI |
| R1122 | RSLRRSSKLVAGLPKDHDGKDDHYLWDIGKKKKDENSVTIRQILTTSA |
| | DTKELKNAGKWREFCEKLGEALYLKSKDMSGGLKITRRILGDAEFHG |
| | KPDRLEKSRSVSIGSVLKETVVCGELVAKTPFFFGAIDEDAKQTDLQVL |
| | LTPDNKYRLPRSAVRGILRRDLQTYFDSPCNAELGGRPCMCKTCRIMR |
| | GITVMDARSEYNAPPEIRHRTRINPFTGTVAEGALFNMEVAPEGIVFPF |
| | QLRYRGSEDGLPDALKTVLKWWAEGQAFMSGAASTGKGRFRMENA |
| | KYETLDLSDENQRNDYLKNWGWRDEKGLEELKKRLNSGLPEPGNYR |
| | DPKWHEINVSIEMASPFINGDPIRAAVDKRGTDVVTFVKYKAEGEEAK |
| | PVCAYKAESFRGVIRSAVARIHMEDGVPLTELTHSDCECLLCQIFGSEY |
| | EAGKIRFEDLVFESDPEPVTFDHVAIDRFTGGAADKKKFDDSPLPGSPA |
| | RPLMLKGSFWIRRDVLEDEEYCKALGKALADVNNGLYPLGGKSAIGY |
| | GQVKSLGIKGDDKRISRLMNPAFDETDVAVPEKPKTDAEVRIEAEKVY |
| | YPHYFVEPHKKVEREEKPCGHQKFHEGRLTGKIRCKLITKTPLIVPDTS |
| | NDDFFRPADKEARKEKDEYHKSYAFFRLHKQIMIPGSELRGMVSSVYE |
| | TVTNSCFRIFDETKRLSWRMDADHQNVLQDFLPGRVTADGKHIQKFSE |
| | TARVPFYDKTQKHFDILDEQEIAGEKPVRMWVKRFIKRGGGSRDSRYQ |
| | KAFQEIPENDPDGWECKEGYLHVVGPSKVEFSDKKGDVINNFQGTLPS |
| | VPNDWKTIRTNDFKNRKRKNEPVFCCEDDKGNYYTMAKYCETFFFDL |
| | KENEEYEIPEKARIKYKELLRVYNNNPQAVPESVFQSRVARENVEKLK |
| | SGDLVYFKHNEKYVEDIVPVRISRTVDDRMIGKRMSADLRPCHGDWV |
| | EDGDLSALNAYPEKRLLLRHPKGLCPACRLFGTGSYKGRVRFGFASLE |
| | NDPEWLIPGKNPGDPFHGGPVMLSLLERPRPTWSIPGSDNKFKVPGRK |
| | FYVHHHAWKTIKDGNHPTTGKAIEQSPNNRTVEALAGGNSFSFEIAFE |
| | NLKEWELGLLIHSLQLEKGLAHKLGMAKSMGFGSVEIDVESVRLRKD |
| | WKQWRNGNSEIPNWLGKGFAKLKEWFRDELDFIENLKKLLWFPEGD |
| | QAPRVCYPMLRKKDDPNGNSGYEELKDGEFKKEDRQKKLTTPWTPW |
| | A |

Table 4 below shows examples of target ssRNA sequences (5′-3′).

TABLE 4

| SEQ ID NO/Description | Sequence |
|---|---|
| SEQ ID NO: 5 | ggAUUACCCAUGUCGAAGACAACAAAG |
| tgRNA for structural analysis | |
| SEQ ID NO: 6 | /5Cy5/GGGGUUGGAAAGCCGGUUUUCUUUGAUG |
| 60-nt processing target | UCACGGAACCUUUGUUGUCUUCGACAUGGGUA |
| SEQ ID NO: 7 | /5Cy5/CCGGCCAUUCAAACAUGAGGAUUACCCAU |
| 60-nt MS2 ssRNA target | GUCGAAGACAACAAAGAAGUUCAACUCUUUA |

Table 5 below shows examples of Cas7-11 guide sequences (5′-3′).

TABLE 5

| SEQ ID NO/Description | Sequence |
|---|---|
| SEQ ID NO: 8 | ggUGAUGUCACGGAACCUUUGUUGUCUUCGACA |
| crRNA for structural analysis | UGGGUAAU |
| SEQ ID NO: 9 | GGUUGAUGUCACGGAACCUUUGUUGUCUUCGA |
| Mature crRNA for MS2 | CAUGGGUAAUCCUCAU |
| ssRNA target | |
| SEQ ID NO: 10 | GGGGUUGGAAAGCCGGUUUUCUUUGAUGUCAC |
| Pre-mature crRNA for MS2 | GGAACCUUUGUUGUCUUCGACAUGGGUAAUCC |
| ssRNA target, WT | UCAU |
| SEQ ID NO: 11 | GGGGUUGGAAAGCCGGUUUUCUUUGAUGUCAC |
| Pre-mature crRNA for MS2 | GGAACGUUUGUUGUCUUCGACAUGGGUAAUCC |
| ssRNA target, mismatch 1 | UCAU |
| SEQ ID NO: 12 | GGGGUUGGAAAGCCGGUUUUCUUUGAUGUCAC |
| Pre-mature crRNA for MS2 | GGAACCAUUGUUGUCUUCGACAUGGGUAAUCC |
| ssRNA target, mismatch 2 | UCAU |
| SEQ ID NO: 13 | GGGGUUGGAAAGCCGGUUUUCUUUGAUGUCAC |
| Pre-mature crRNA for MS2 | GGAACCUAUGUUGUCUUCGACAUGGGUAAUCC |
| ssRNA target, mismatch 3 | UCAU |
| SEQ ID NO: 14 | GGGGUUGGAAAGCCGGUUUUCUUUGAUGUCAC |
| Pre-mature crRNA for MS2 | GGAACCUUAGUUGUCUUCGACAUGGGUAAUCC |
| ssRNA target, mismatch 4 | UCAU |
| SEQ ID NO: 15 | GGGGUUGGAAAGCCGGUUUUCUUUGAUGUCAC |
| Pre-mature crRNA for MS2 | GGAACCUUUCUUGUCUUCGACAUGGGUAAUCC |
| ssRNA target, mismatch 5 | UCAU |
| SEQ ID NO: 16 | GGGGUUGGAAAGCCGGUUUUCUUUGAUGUCAC |
| Pre-mature crRNA for MS2 | GGAACCUUUGAUGUCUUCGACAUGGGUAAUCC |
| ssRNA target, mismatch 6 | UCAU |
| SEQ ID NO: 17 | GGGGUUGGAAAGCCGGUUUUCUUUGAUGUCAC |
| Pre-mature crRNA for MS2 | GGAACCUUUGUAGUCUUCGACAUGGGUAAUCC |
| ssRNA target, mismatch 7 | UCAU |
| SEQ ID NO: 18 | GGGGUUGGAAAGCCGGUUUUCUUUGAUGUCAC |
| Pre-mature crRNA for MS2 | GGAACCUUUGUUCUCUUCGACAUGGGUAAUCC |
| ssRNA target, mismatch 8 | UCAU |
| SEQ ID NO: 19 | GGGGUUGGAAAGCCGGUUUUCUUUGAUGUCAC |
| Pre-mature crRNA for MS2 | GGAACCUUUGUUGACUUCGACAUGGGUAAUCC |
| ssRNA target, mismatch 9 | UCAU |
| SEQ ID NO: 20 | GGGGUUGGAAAGCCGGUUUUCUUUGAUGUCAC |
| Pre-mature crRNA for MS2 | GGAACCUUUGUUGUGUUCGACAUGGGUAAUCC |
| ssRNA target, mismatch 10 | UCAU |
| SEQ ID NO: 21 | GGGGUUGGAAAGCCGGUUUUCUUUGAUGUCAC |
| Pre-mature crRNA for MS2 | GGAACCUUUGUUGUCAUCGACAUGGGUAAUCC |
| ssRNA target, mismatch 11 | UCAU |
| SEQ ID NO: 22 | GGGGUUGGAAAGCCGGUUUUCUUUGAUGUCAC |
| Pre-mature crRNA for MS2 | GGAACCUUUGUUGUCUACGACAUGGGUAAUCC |
| ssRNA target, mismatch 12 | UCAU |
| SEQ ID NO: 23 | GGGGUUGGAAAGCCGGUUUUCUUUGAUGUCAC |
| Pre-mature crRNA for MS2 | GGAACCUUUGUUGUCUUGGACAUGGGUAAUCC |
| ssRNA target, mismatch 13 | UCAU |
| SEQ ID NO: 24 | GGGGUUGGAAAGCCGGUUUUCUUUGAUGUCAC |
| Pre-mature crRNA for MS2 | GGAACCUUUGUUGUCUUCCACAUGGGUAAUCC |
| ssRNA target, mismatch 14 | UCAU |
| SEQ ID NO: 25 | GGGGUUGGAAAGCCGGUUUUCUUUGAUGUCAC |
| Pre-mature crRNA for MS2 | GGAACCUUUGUUGUCUUCGUCAUGGGUAAUCC |
| ssRNA target, mismatch 15 | UCAU |
| SEQ ID NO: 26 | GGGGUUGGAAAGCCGGUUUUCUUUGAUGUCAC |
| Pre-mature crRNA for MS2 | GGAACCUUUGUUGUCUUCGAGAUGGGUAAUCC |
| ssRNA target, mismatch 16 | UCAU |
| SEQ ID NO: 27 | GGGGUUGGAAAGCCGGUUUUCUUUGAUGUCAC |
| Pre-mature crRNA for MS2 | GGAACCUUUGUUGUCUUCGACUUGGGUAAUCC |
| ssRNA target, mismatch 17 | UCAU |
| SEQ ID NO: 28 | GGGGUUGGAAAGCCGGUUUUCUUUGAUGUCAC |
| Pre-mature crRNA for MS2 | GGAACCUUUGUUGUCUUCGACAAGGGUAAUCC |
| ssRNA target, mismatch 18 | UCAU |
| SEQ ID NO: 29 | GGGGUUGGAAAGCCGGUUUUCUUUGAUGUCAC |
| Pre-mature crRNA for MS2 | GGAACCUUUGUUGUCUUCGACAUCGGUAAUCC |
| ssRNA target, mismatch 19 | UCAU |
| SEQ ID NO: 30 | GGGGUUGGAAAGCCGGUUUUCUUUGAUGUCAC |
| Pre-mature crRNA for MS2 | GGAACCUUUGUUGUCUUCGACAUGCGUAAUCC |
| ssRNA target, mismatch 20 | UCAU |
| SEQ ID NO: 31 | GGGGUUGGAAAGCCGGUUUUCUUUGAUGUCAC |
| Pre-mature crRNA for MS2 | GGAACCUUUGUUGUCUUCGACAUGGCUAAUCC |
| ssRNA target, mismatch 21 | UCAU |
| SEQ ID NO: 32 | GGGGUUGGAAAGCCGGUUUUCUUUGAUGUCAC |
| Pre-mature crRNA for MS2 | GGAACCUUUGUUGUCUUCGACAUGGGAAAUCC |
| ssRNA target, mismatch 22 | UCAU |
| SEQ ID NO: 33 | GGGGUUGGAAAGCCGGUUUUCUUUGAUGUCAC |
| Pre-mature crRNA for MS2 | GGAACCUUUGUUGUCUUCGACAUGGGUUAUCC |
| ssRNA target, mismatch 23 | UCAU |
| SEQ ID NO: 34 | GGGGUUGGAAAGCCGGUUUUCUUUGAUGUCAC |
| Pre-mature crRNA for MS2 | GGAACCUAUGUUGACUUCGACAUGGGUAAUCC |
| ssRNA target, mismatch 3/9 | UCAU |
| SEQ ID NO: 35 | GUUGAUGUCACGGAACUGCAGCCAGCUUUCCG |
| Gluc targeting guide, WT | GGCAUUGGCUUCCAU |
| SEQ ID NO: 36 | GUUGAUGUCACGGAACGGUAAUGCCUGGCUUG |
| Non-targeting guide | UCGACGCAUAGUCUG |
| SEQ ID NO: 37 | GUUGAUGUCACGGAACCAGUGUUGGUAGGAGU |
| PPIB targeting guide | UUGUUACAAAAGUGA |
| SEQ ID NO: 38 | GUUGAUGUCACGGAACGGUUAUAGCUUGACAA |
| MALAT1 targeting guide | GCAAUUAACUUUAAA |

### Example 1

### Protein and RNA Preparation

A gene encoding D. ishimotonii Cas7-11 (residues 1-1601) was amplified by PCR and cloned into the modified pET vector (Novagen), in which Cas7-11 has an N-terminal maltose-binding protein (MBP) and a C-terminal His₆-tag. The two inactivating mutations (D429A/D654A) were introduced into Cas7-11 by a PCR-based method, and the sequence was confirmed by DNA sequencing. The MBP-Cas7-11 (D429A/D654A)-His₆ protein was expressed in Escherichia coli Rosetta2 (DE3) (Novagen) by inducing with 0.1 mM isopropyl β-D-thiogalactopyranoside (Nacalai Tesque) at 20° C. overnight. The E. coli cells were lysed by sonication and the lysate was clarified by centrifugation. The supernatant was applied to Ni-NTA superflow (QIAGEN) and the MBP-Cas7-11 (D429A/D654A)-His₆ protein was eluted by buffer A (20 mM Tris-HCl, pH 8.0, 20 mM imidazole, 1 M NaCl, 3 mM 2-mercaptoethanol, and 1 mM phenylmethylsulfonyl fluoride) with 300 mM imidazole. The protein was further purified by chromatography on Amyrose resin (NEB), HiTrap Heparin (GE Healthcare), and HiLoad 16/600 Superdex 200 (GE Healthcare) columns. The crRNA (39 nucleotides plus 5′ GG for in vitro transcription) and target RNA (25 nucleotides plus 5′ GG for in vitro transcription) were transcribed in vitro with T7 RNA polymerase and purified by 10% denaturing (7 M urea) polyacrylamide gel electrophoresis. The purified materials were stored at −80° C. until use.

### Example 2

### Cryo-EM Grid Preparation and Data Collection

A Cas7-11-crRNA-target RNA complex was reconstituted by mixing the purified MBP-Cas7-11 protein, the 39-nucleotide crRNA, and the 25-nucleotide target RNA, at a molar ratio of 1:1.2:1.5. The complex was purified by size-exclusion chromatography on a Superose6 Increase 10/300 column (GE Healthcare), equilibrated with the buffer containing 20 mM Hepes-NaOH, pH 7.0, 150 mM NaCl, 2 mM MgCl₂, and 1 mM DTT. The peak fraction containing Cas7-11-:crRNA-target RNA complex was concentrated to 1.5 A260 units using an Amicon Ultra-4 filter (10 kDa molecular-weight cutoff; Millipore). The samples (3 μl) were then applied to freshly glow-discharged Au 300 mesh R1.2/1.3 grids (Quantifoil) in a Vitrobot Mark IV (FEI) at 4° C. with a waiting time of 10 sec and a blotting time of 4 sec under 100% humidity conditions. The grids were plunge-frozen into liquid ethane cooled at liquid nitrogen temperature.

The cryo-EM data were collected using a Titan Krios G3i microscope (Thermo Fisher Scientific), running at 300 kV and equipped with a Gatan Quantum-LS Energy Filter (GIF) and a Gatan K3 Summit direct electron detector. Micrographs were recorded at a nominal magnification of ×105,000 with a pixel size of 0.83 Å in a total exposure of 52 e⁻/A² per 64 frames by the correlated double sampling mode. The data were automatically acquired by the image shift method using the SerialEM software (Mastronarde, 2005), with a defocus range of −0.8 to −1.6 μm, and 2,781 movies were acquired.

### Example 3

### Image Processing

Data processing was performed using a combination of cryoSPARC v3.2.0 and Relion3.1 software packages. The dose-fractionated movies were aligned using the Patch motion correction and the contrast transfer function (CTF) parameters were estimated using Patch-Based CTF estimation in cryoSPARC. Particles were automatically picked using Blob picker in cryoSPARC followed by multiple times of reference free 2D classification to curate particle sets. The particles were further curated by cryoSPARC Heterogeneous Refinement (N=6) using the map derived from cryoSPARC Ab initio Reconstruction as a template. The best class containing 581,179 particles was refined using Homogeneous refinement followed by non-uniform refinement, yielding a map at 2.46 Å resolution. To improve the quality of 3D reconstruction in the leg domain, the particles were imported into Relion and subjected to 3D classification without alignment using a mask for the leg domain. The selected 135,109 particles were imported back to cryoSPARC and non-uniform refinement after local motion correction yielded a map at 2.45 Å resolution, according to the Fourier shell correlation (FSC)=0.143 criterion. The local resolution was estimated by BlocRes in cryoSPARC.

### Example 4

### Model Building and Validation

A model was built using Nautilus and Buccaneer in CCP-EM package and manually built using COOT against the density map sharpened using DeepEMhancer. The model was refined using Real-space refinement in PHENIX with the secondary structure restraints. The structure validation was performed using MolProbity from the PHENIX package. The curve representing model vs. full map was calculated using phenix.mtriage, based on the final model and the full, filtered, and sharpened map. The statistics of the 3D reconstruction and model refinement are summarized in Table 6. The cryo-EM density maps were calculated with UCSF ChimeraX, and molecular graphics figures were prepared with CueMol (http://www.cuemol.org).

TABLE 6
Data Collection and Structural Refinement

| Data collection and processing | |
|---|---|
| Magnification | 105,000 |
| Voltage (kV) | 300 |
| Electron exposure (e⁻/Å²) | 76.5/72.8 |
| Defocus range (μm) | −0.6 to −1.5 |
| Pixel size (Å) | 0.83 |
| Initial particle images (no.) | 4,452,943 |
| Refinement | |
| PDB ID | 7WAH |
| EMDB ID | EMD-32385 |
| Final particle images (no.) | 135,109 |
| Map resolution (Å) | 2.45 |
| FSC threshold | 0.143 |
| Model resolution (Å) | 2.89 |
| FSC threshold | 0.5 |
| Map sharpening B factor (Å²) | −46.8 |
| Model composition | |
| Non-hydrogen atoms | 13,228 |
| Protein residues | 1,473 |
| RNA nucleotides | 62 |
| Ligands | 4 |
| R.m.s. deviations | |
| Bond lengths (Å) | 0.002 |
| Bond angles (°) | 0.507 |
| Validation | |
| Clashscore | 8.27 |
| Poor rotamers (%) | 0.47 |
| Ramachandran plot (%) | |
| Favored | 97.32 |
| Allowed | 2.68 |
| Outliers | 0.00 |

### Example 5

### In Vitro Pre-crRNA and Target RNA Cleavage Assays

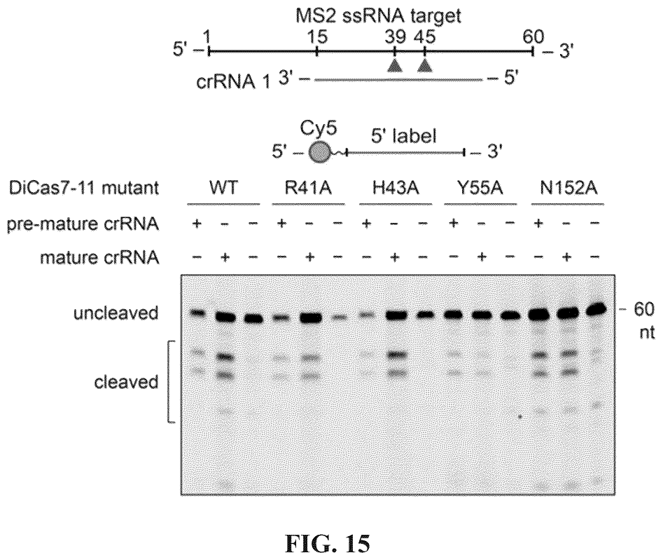

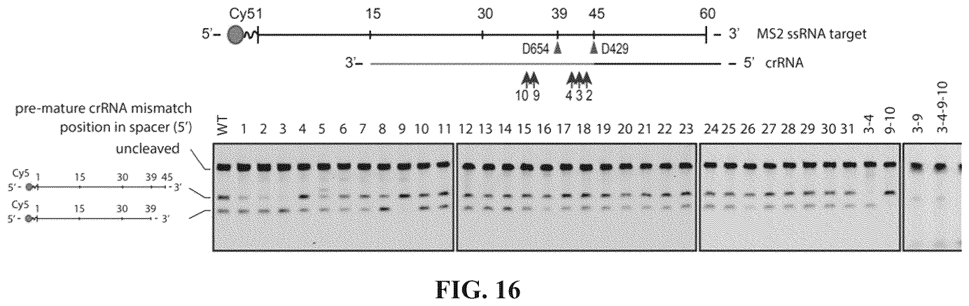

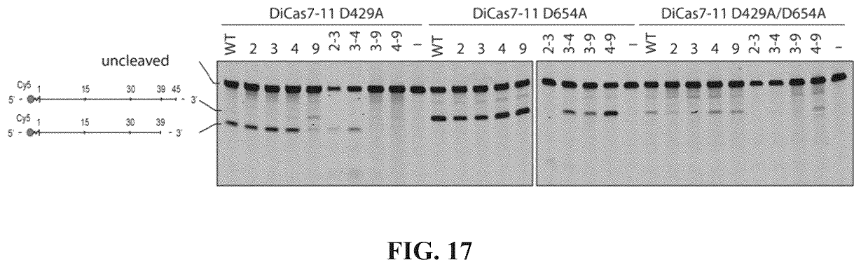

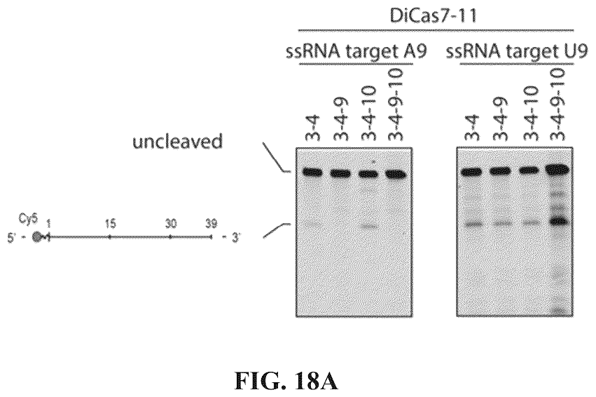

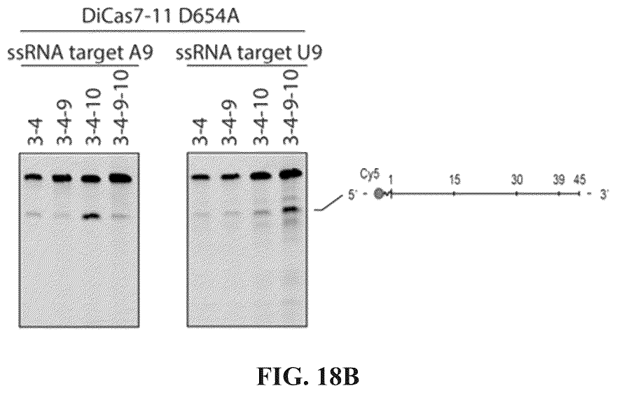

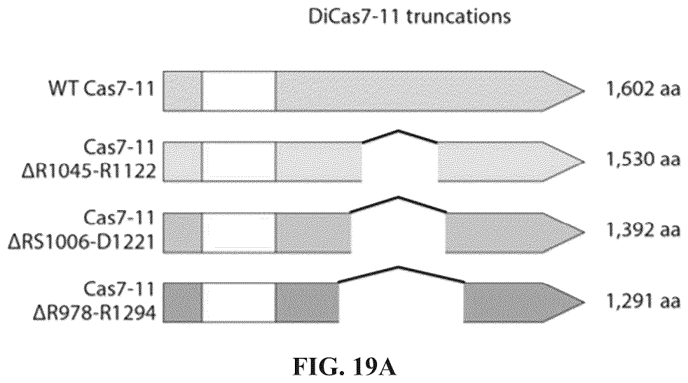

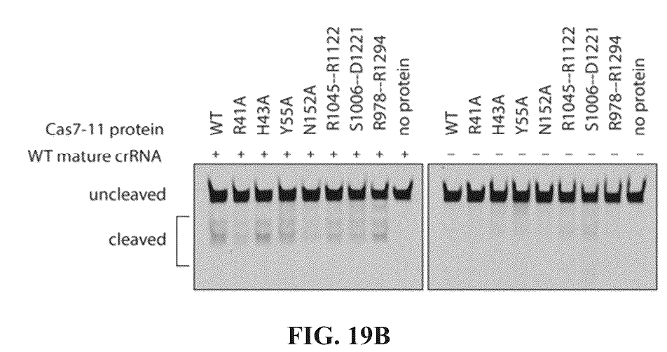

In vitro Pre-crRNA and target RNA cleavage assays were performed (FIGS. 15-19). 5′-labelled ssRNA processing and cleavage targets were synthesized by Integrated DNA Technologies. crRNA and unlabeled ssRNA targets were prepared with the HiScribe™ T7 High Yield RNA Synthesis Kit (New England Biolabs). In vitro transcription products were then purified with either RNAClean XP magnetic beads (Beckman Coulter) or RNA Clean and Concentrator columns (R1017, Zymo Research). In vitro cleavage assays were performed with 233 nM purified Cas7-11, 30 nM of 5′-Cy5-labelled ssRNA targets and 200 nM crRNA in nuclease assay buffer (40 mM Tris-HCl, pH 7.5, 60 mM NaCl and 6 mM MgCl₂) supplemented with 4 U of RNase inhibitor, murine (M0314S, New England Biolabs). For pre-crRNA processing reactions, crRNA was omitted, and pre-crRNA was used in place of the labelled ssRNA target. Reactions were incubated for 1 h at 37° C. (unless otherwise indicated) and then quenched with addition of proteinase K, EDTA and urea (final concentrations 1 mg ml⁻¹ proteinase K, 6 mM EDTA and 400 μM urea) for 30 min at 50° C. Reactions were denatured with 4.5 M urea denaturing buffer at 95° C. for 5 min, and loaded onto a 10% (for pre-crRNA processing) or 6% (for target ssRNA cleavage) Novex PAGE Tris-borate-EDTA (TBE)-urea gel (EC6885BOX, Invitrogen), which was run at 200 V for 35 min at 60° C. Gels were imaged using an Odyssey scanner (LI-COR Biosciences).

### Example 6

### Mammalian Cell Culture

Mammalian experiments were performed using the HEK293FT cell line, acquired from and authenticated by American Type Culture Collection (ATCC). HEK293FT cells were grown in Dulbecco's modified Eagle medium with high glucose, sodium pyruvate and GlutaMAX (Thermo Fisher Scientific), additionally supplemented with 1× penicillin-streptomycin (Thermo Fisher Scientific) and 10% fetal bovine serum (Thermo Fisher Scientific) and passaged using TrypLE Express (Thermo Fisher Scientific). Cells were maintained at 37° C. and 5% CO₂. For transfection of HEK293FT cells, cells were plated 16 h before transfection at seeding densities of 1.5×10⁴ cells per well in a 96-well plate or 1.5×10⁶ cells per T25 flask, allowing cells to reach 90% confluency by transfection. Cells were then transfected with Lipofectamine 3000 (Thermo Fisher Scientific) following the manufacturer's protocol with 200 ng total plasmid per well in a 96-well plate and 7.7 μg total plasmid in a T25 flask.

### Example 7

### RNA Knockdown Assay in Human Cells

To assess RNA knockdown in mammalian cells with reporter constructs, 80 ng of the DiCas7-11 expression vector was co-transfected with 80 ng of guide expression plasmid and 40 ng of the dual luciferase reporter. After 48 h, the medium containing the secreted luciferase was collected and luciferase activity was measured using the Gaussia Luciferase Assay reagent (GAR-2B; Targeting Systems) and Cypridina (Vargula) luciferase assay reagent (VLAR-2; Targeting Systems) kits. Assays were performed in white 96-well plates on a plate reader (Biotek Synergy Neo 2) with an injection protocol. All replicates performed were biological replicates. Luciferase measurements were normalized by dividing the Gluc values by the Cluc values, thus normalizing for any variation between wells.

For targeting of endogenous genes, 100 ng of the DiCas7-11 expression vector was co-transfected with 100 ng of guide expression plasmid. After 48 h, the cells were lysed and the RNA were collected using a previously described method (Julia Joung, Nat. Protocol) with the Revertaid RT Reverse Transcription Kit (Thermo Fisher), following the manufacturer's protocol. Using Fast Advanced Master Mix (Thermo Fisher Scientific), gene expression was measured using the cDNA by TaqMan qPCR probes for the KRAS, PPIB, and MALAT1 transcripts (Thermo Fisher Scientific) as well as the GAPDH control probe (Thermo Fisher Scientific). qPCR reactions were read out on a Bio-Rad CFX384 Touch Real-Time PCR Detection System, with three 10-μl technical replicates in 384-well format. No statistical methods were used for determining sample size. No blinding or randomization methods were used during these experiments.

### Example 8

### Truncated DiCas7-11 AAV Production and Delivery

AAV was prepared by designing vectors with truncated Cas7-11 expression using an EFS promoter or guide expression with a U6 or tRNA promoter. HEK293FT cells were transfected in T25 flasks using Lipofectamine 3000 (Thermo Fisher Scientific) with 2.0 μg of cargo plasmid, 1.8 μg AAV8 capsid vector and 3.9 μg AAV helper pAdDeltaF6 plasmid (Addgene 112867) per T25 flask according to the manufacturer's protocol. Two days after the transfection, the medium containing the loaded viral vector was filtered using a 0.45-μm filter (Sigma Aldrich), concentrated by an Amicon Ultra-15 Centrifugal Filter Unit (MWCO 100 kDa), washed once with 1×DPBS (Thermo Fisher), and the final product was stored at −80° C. To evaluate knockdown on the luciferase reporter expressed in HEK293FT cells, AAV was added at varying titres to the 40,000 cells per well in a 96-well plate by spinfection at 2,000 g and 37° C. for 2 h. The dual luciferase reporter plasmid was subsequently transfected to the HEK293FT cells at 100 ng per well with Lipofectamine 3000. Cell media was harvested for luciferase chemiluminescence measurement 48 h later. AAV genome titer was determined by RT-qPCR, using a pair of primers targeting the EFS promoter in the Fast SYBR Green Master Mix (Applied Biosystems).

### Example 9

### Structure of Cas7-11 Effector Complex

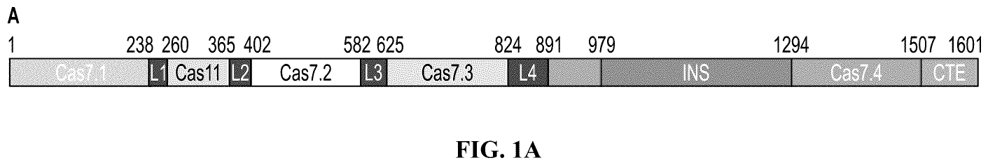

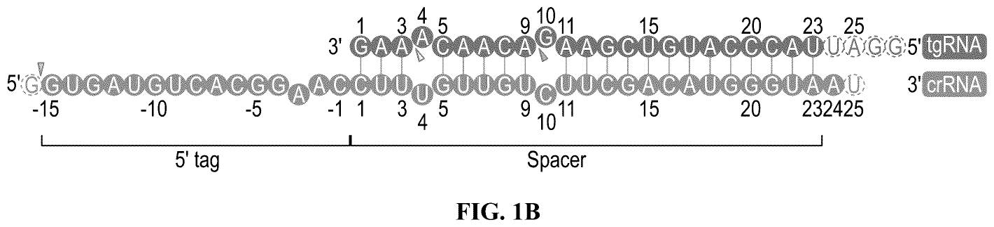

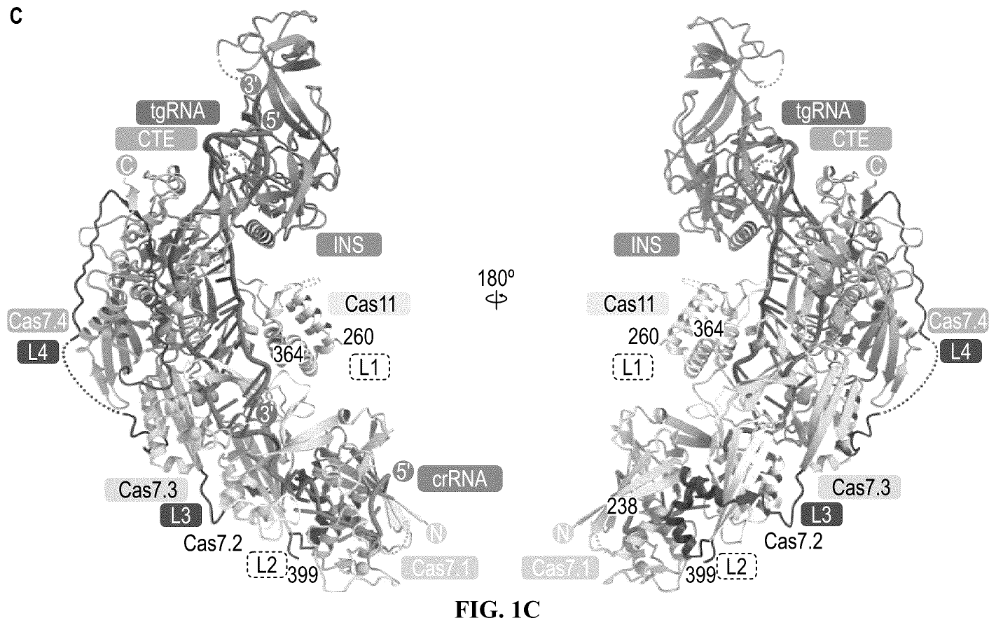

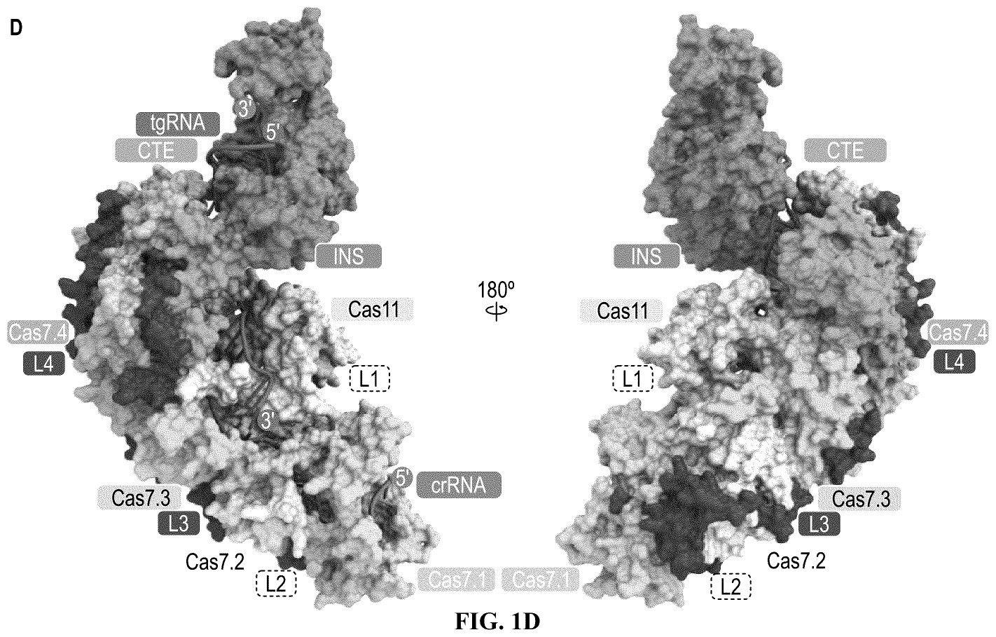

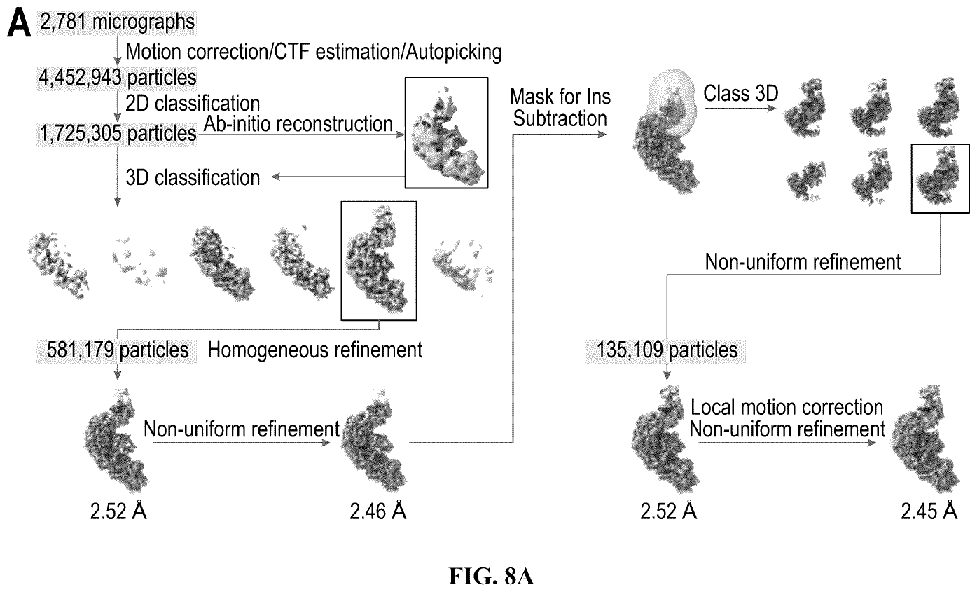

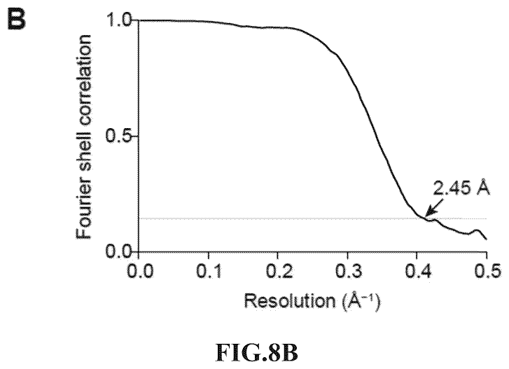

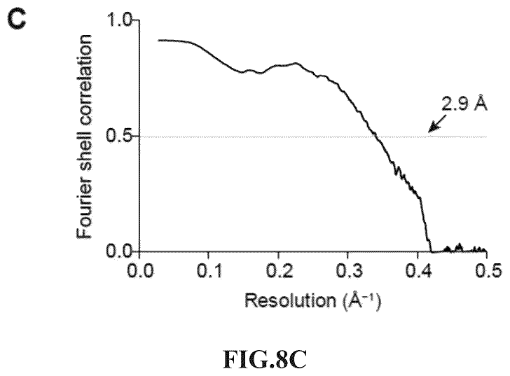

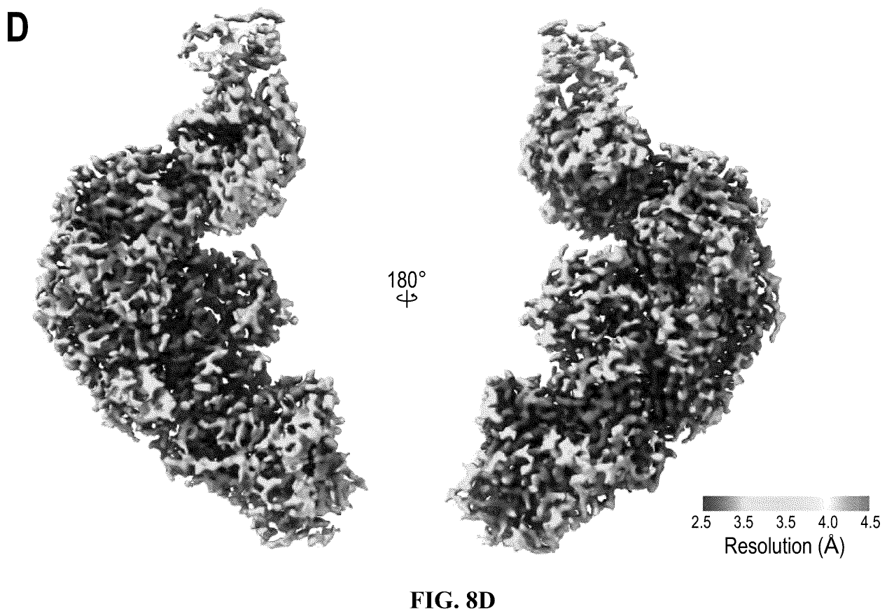

The cryo-EM structure of D. ishimotonii Cas7-11, catalytically inactivated by D429A/D654A mutations, in complex with a 39-nt crRNA (U(−14)-U25) and its complementary 25-nt target RNA (G1*-A25*) at 2.5-A resolution were determined (FIGS. 1A, 1B, and 8A-8D).

Cas7-11 consists of four Cas7 domains (Cas7.1-Cas7.4), and a Cas11 domain, interspaced with four interdomain linkers (L1-L4), with Cas7.4 harboring an additional large insertion (INS)(residues 979-1293) domain and a C-terminal extension (CTE)(residues 1507-1601) domain (FIGS. 1C and 1D). Cas7-11 has a baby-like structure, with Cas7.1, Cas7.2-Cas7.4/CTE, INS, and Cas11 corresponding to the head, body, legs, and arms, respectively. The Cas7.1-Cas7.4 domains stack and form a right-handed helical filament. Cas11 interacts with Cas7.2 and Cas7.3 at the midpoint and is directly connected with Cas7.1 and Cas7.2 by L1 (residues 238-259) and L2 (residues 365-401) which, are disordered in the present structure, indicating their flexibility. The repeat-derived region (referred to as the 5′ tag) of the crRNA is anchored by Cas7.1 and Cas7.2, while the duplex formed by the spacer-derived region of the crRNA and target RNA is recognized by Cas7.2-Cas7.4 and INS. CTE extensively interacts with Cas7.3, Cas7.4, and L4, structurally reinforcing the Cas7-11 architecture.

### Example 10

### Domain Structure of Cas7-11

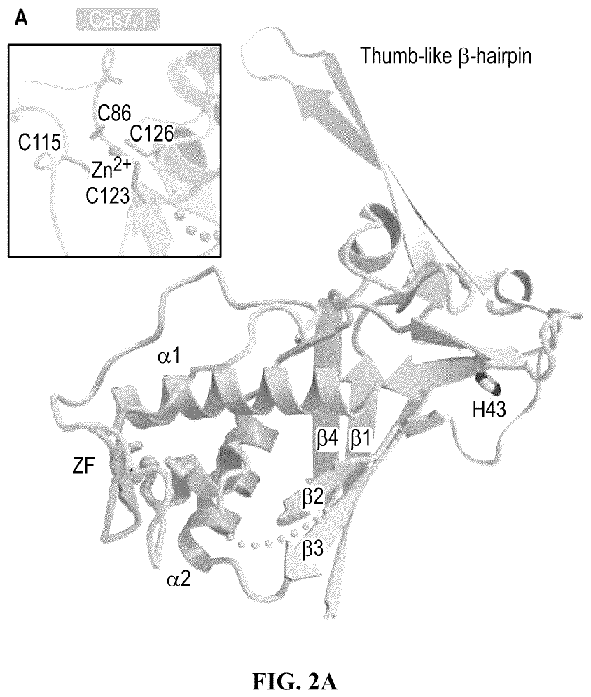

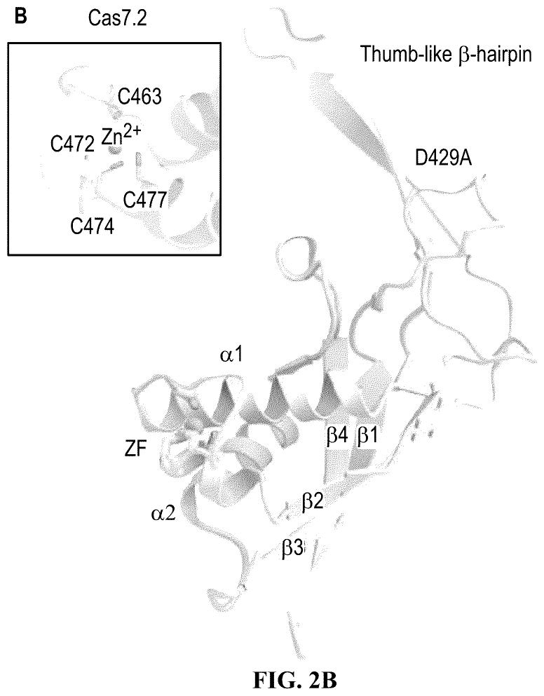

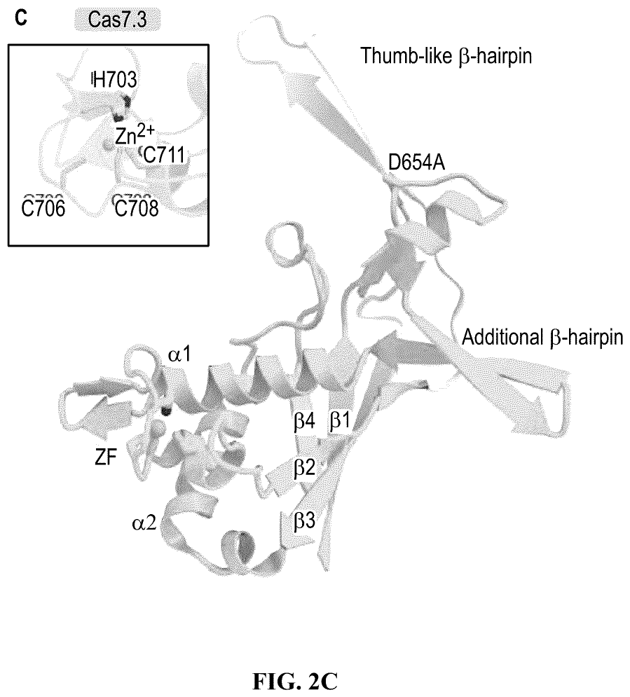

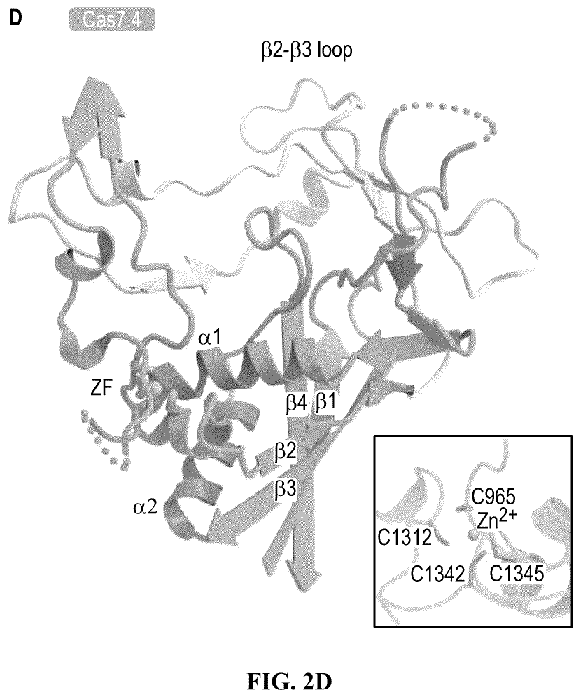

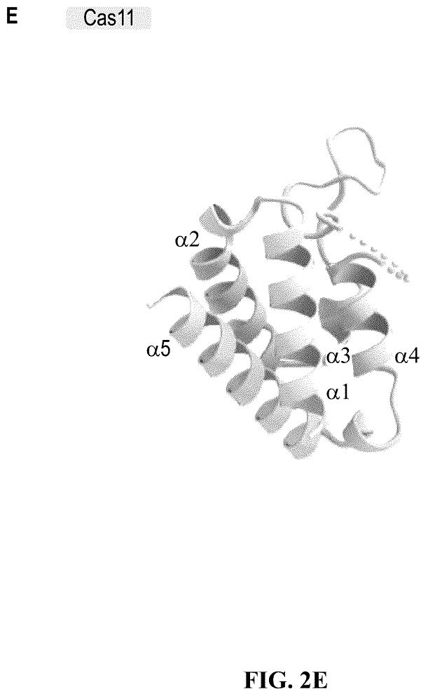

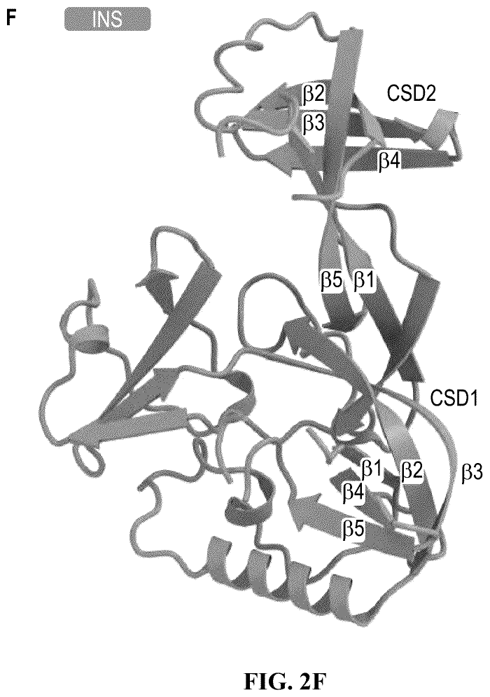

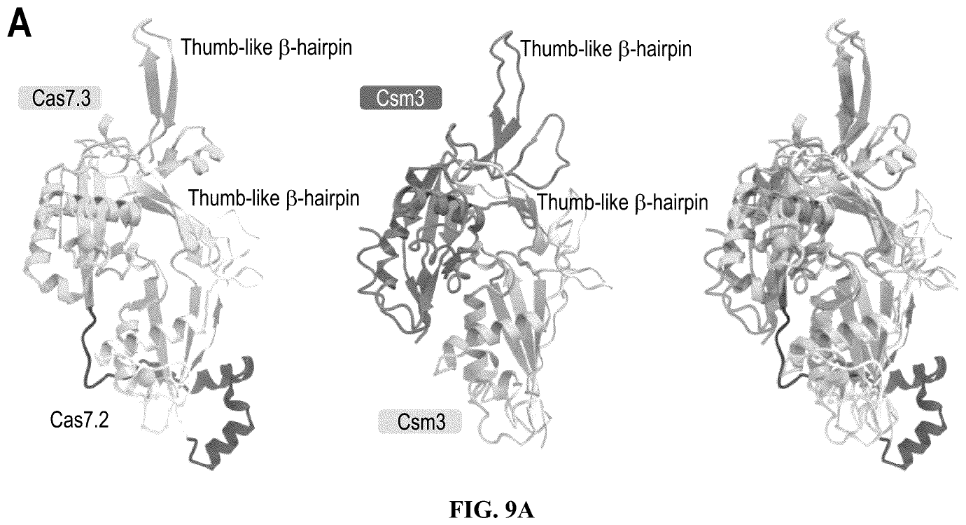

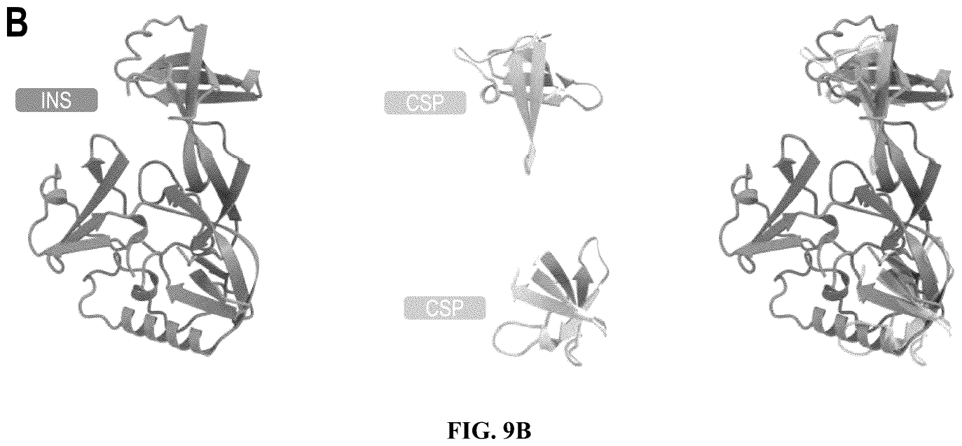

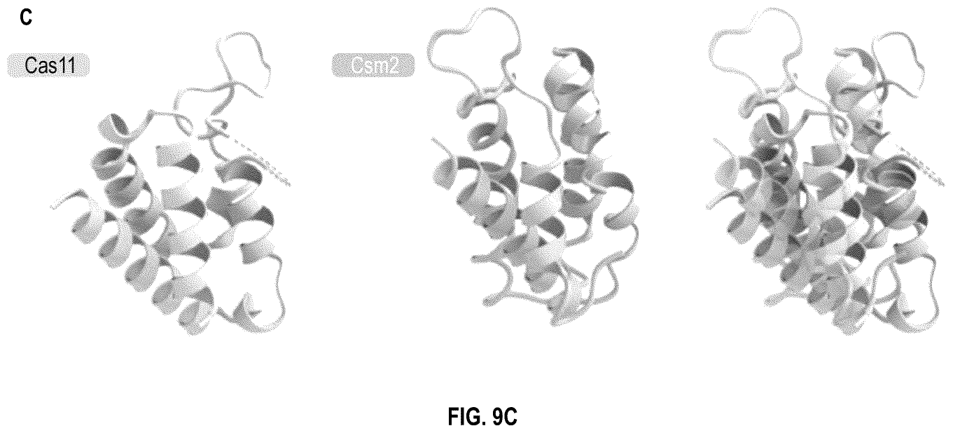

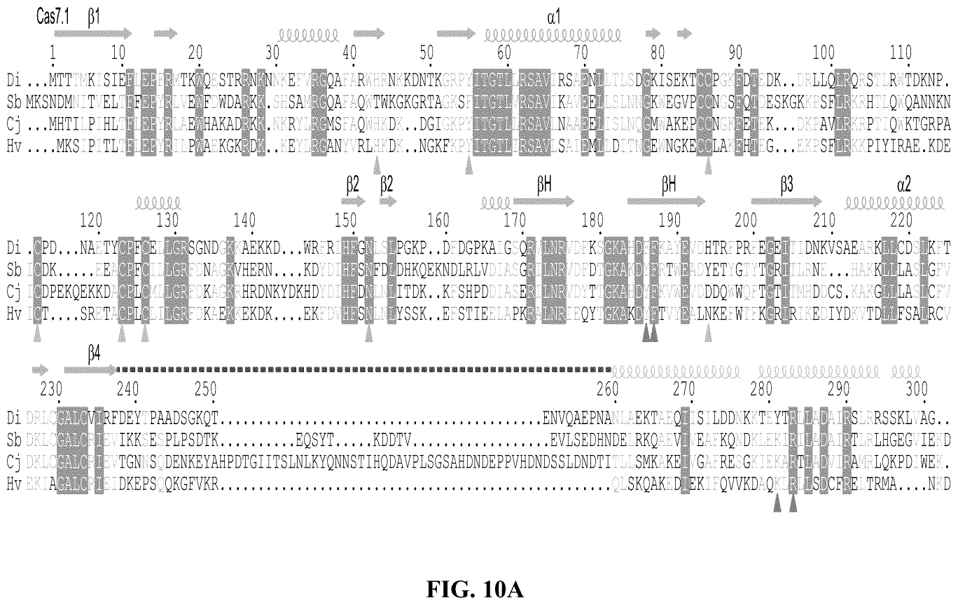

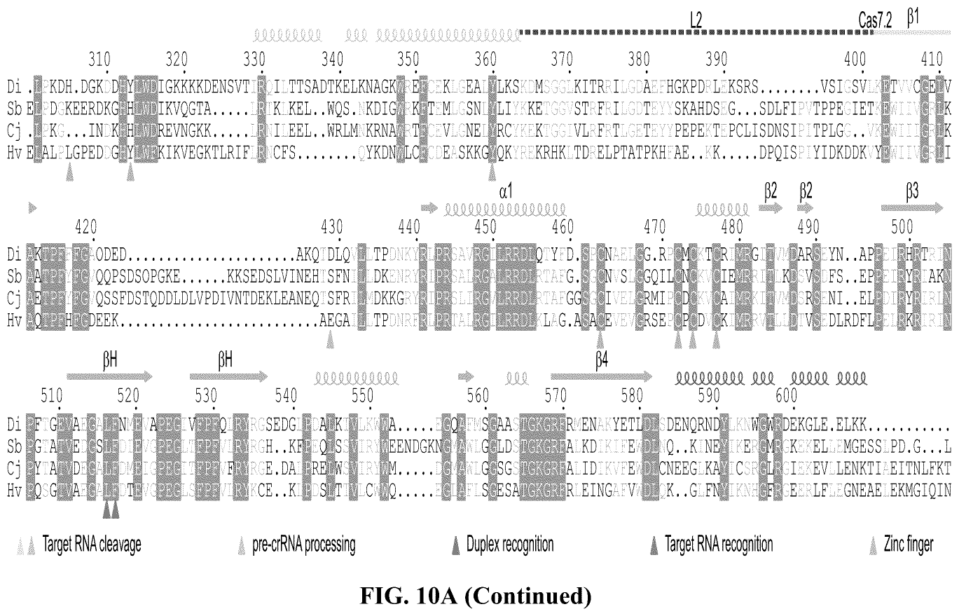

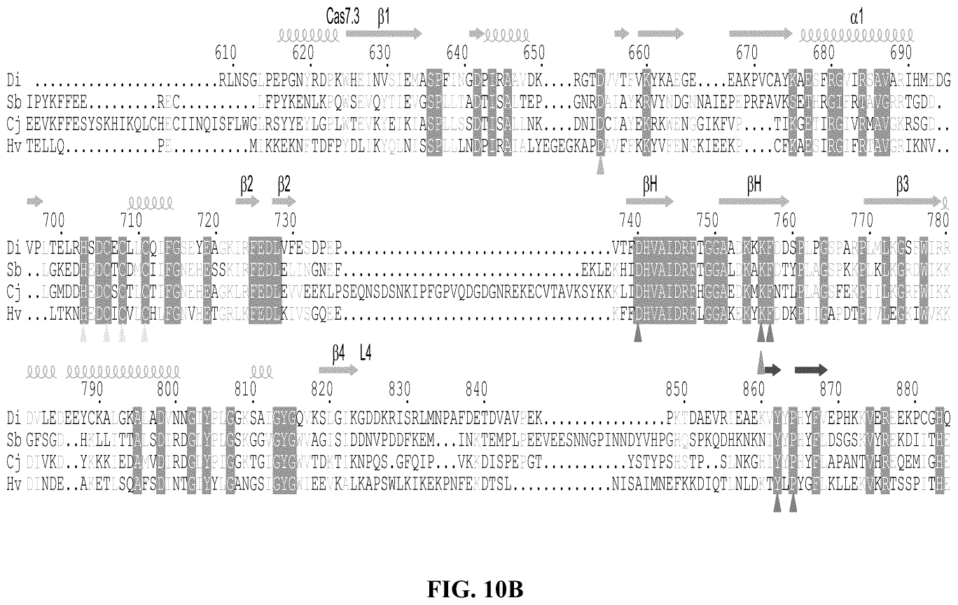

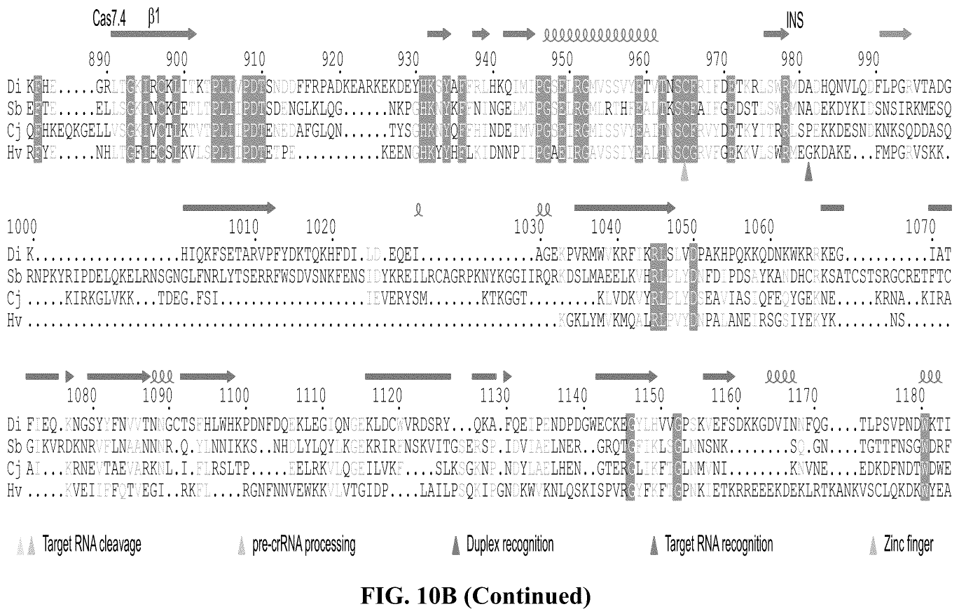

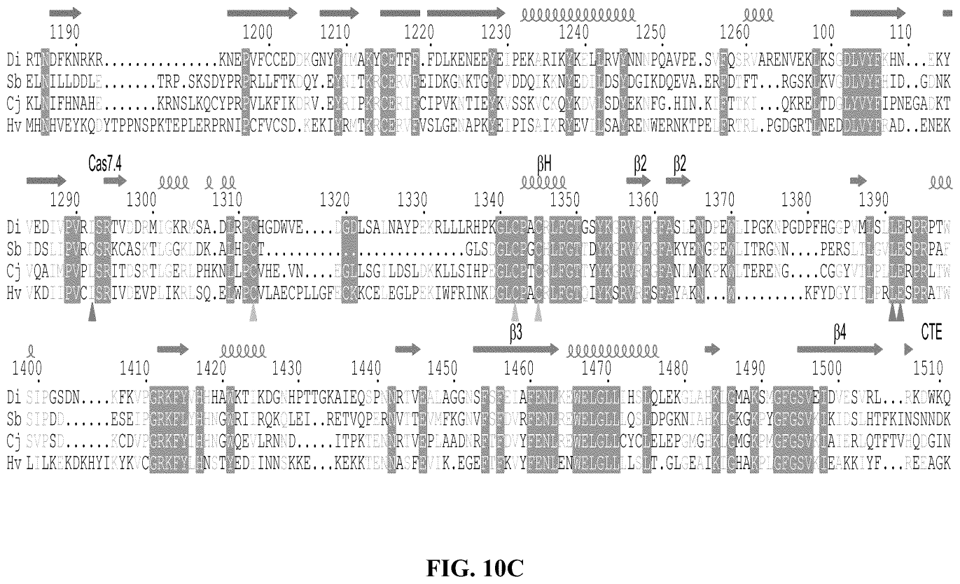

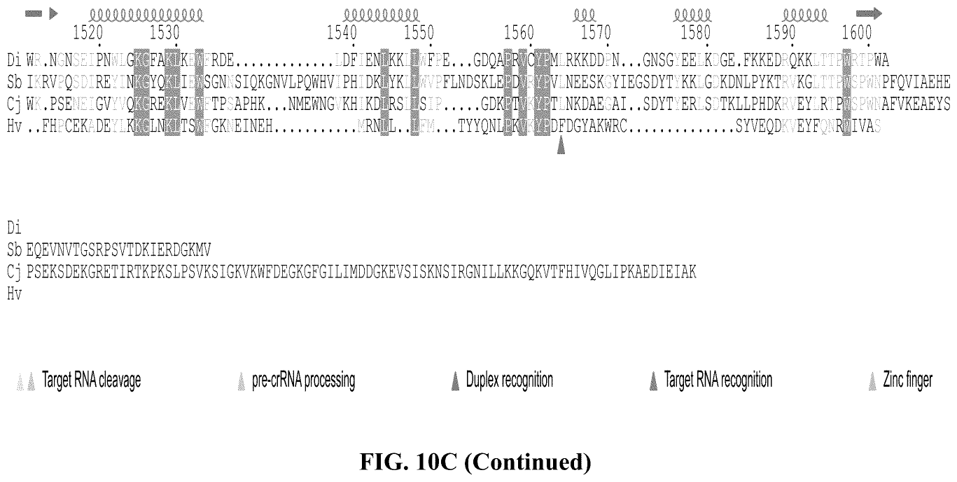

The domain structure of Cas7-11 effector was assessed. The Cas7.1-Cas7.4 domains contain a modified RRM (RNA recognition motif) fold (also known as a ferredoxin-like fold), consisting of a four-stranded antiparallel β-sheet flanked by two α helices in a βαββαβ topology, as commonly observed in the type III-A/B Cas7 proteins (Csm3/Cmr4) (Taylor et al. 2015; Osawa et al. 2015; You et al. 2019; and Jia et al. 2019, incorporated herewith in their entirety by reference) (FIGS. 2A-2E and 1A). Unexpectedly, unlike Csm3 and Cmr4, Cas7.1-Cas7.4 contain a zinc finger motif between the α1 helix and β2 strand in the RRM fold, with zinc ions in each of Cas7.1-Cas7.4 coordinated by C86/C115/C123/C126, C463/C472/C474/C477, H703/C706/C708/C711, and C965/C1312/C1342/C1345, respectively (FIGS. 2A-2D). These zinc-coordinating residues are highly conserved among the Cas7-11 orthologs (FIGS. 10A-10C), indicating that zinc fingers are shared structural features of the Cas7-11 proteins. These residues are not conserved in the Cas7.1 and Cas7.3 subunits of the Cas7×3 family (homologous to the Cas7.2 and Cas7.4 domains of Cas7-11, respectively), but are present in a subset of the Cas7.2 subunit of Cas7×3, implying the zinc finger insertion succeeded the fusion event that produced the Cas7×3 family. Cas7.1-Cas7.3 have their RRM fold with a thumb-like β-hairpin between the β2 and β3 strands, as observed in Csm3 and Cmr4 (FIGS. 2A-2C and 9A). Cas7.1-Cas7.3 also contain unique structural elements, consistent with their distinct functional roles, such as pre-crRNA processing and target RNA cleavage. Cas7.1 and Cas7.2/Cas7.3 possess the catalytic residues for pre-crRNA processing (H43) and target RNA cleavage (D429 and D654) between the β1 strand and the α1 helix, respectively. Unlike Cas7.1 and Cas7.2, Cas7.3 has an additional β-hairpin between the β1strand and the α1 helix (FIG. 2C). In line with its later fusion into the single effector during the evolution of Cas7-11, Cas7.4 adopts the RRM fold highly divergent from those of Cas7.1-Cas7.3 and contains a larger insertion (residues 1365-1452), rather than a thumb-like β-hairpin, between the β2 and β3 strands (FIG. 2D). In addition, the INS domain (residues 966-1311) is inserted within the zinc finger motif of Cas7.4. The Cas11 domain adopts a five-helix bundle similar to the other type III Cas11 proteins (Csm2 and Cmr5) (FIGS. 2E and 9B).

The INS domain comprises two five-stranded β-barrels and additional structural elements, including an a helix and a four-stranded antiparallel β-sheet (FIGS. 2F and 9C). Computational structural comparison revealed that the two β-barrels of INS are structurally similar to cold shock proteins, such as CspB, and the rest of INS lacks structural similarity with any other known proteins (e.g., Lisa Holm, “Using Dali for Protein Structure Comparison,” Methods Mol. Biol. 2020; 2112:29-42, doi 10.1007/978-1-0716-0270-6_3; and Schindelin, H., Marahiel, M. & Heinemann, U., “Universal nucleic acid-binding domain revealed by crystal structure of the B. subtilis major cold-shock protein,” Nature 364, 164-168 (1993), doi 10.1038/364164a0, incorporated herewith in their entirety by reference). The INS domain is less resolved in the density map relative to the other domains (FIG. 8D), suggesting its flexibility, consistent with limited contacts between the INS domain and the rest of the Cas7-11 domains.

### Example 11

### Domain Interfaces of Cas7-11

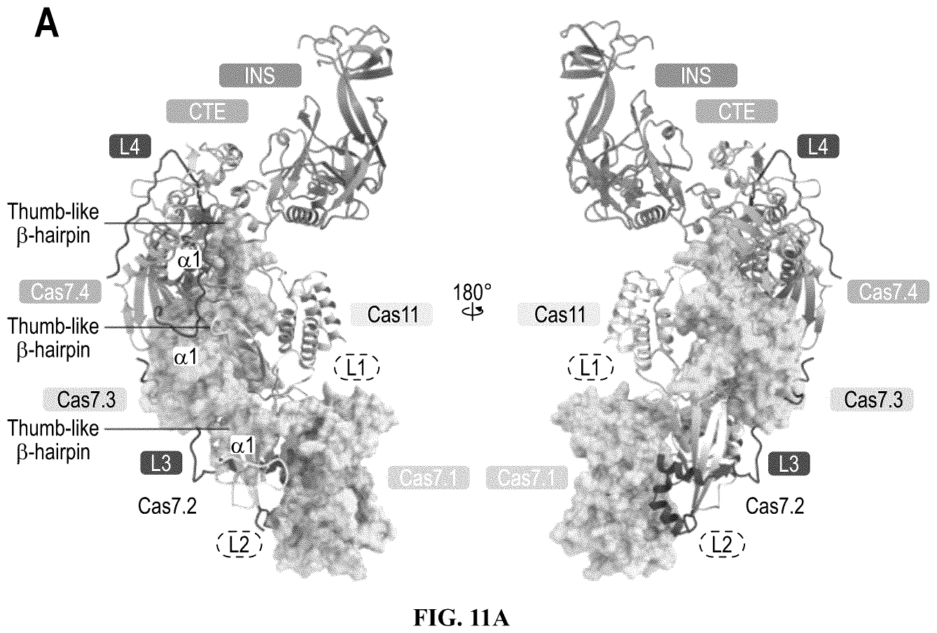

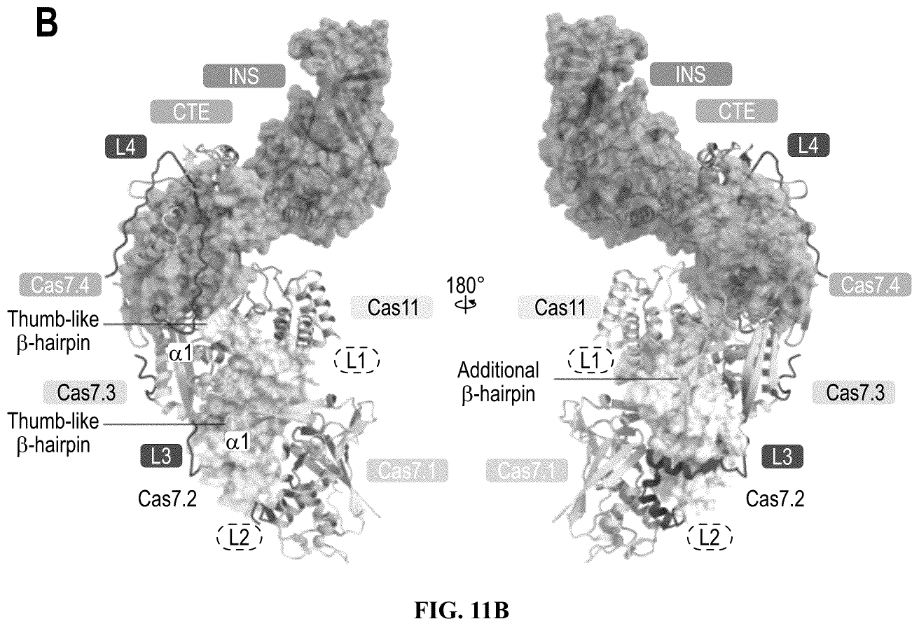

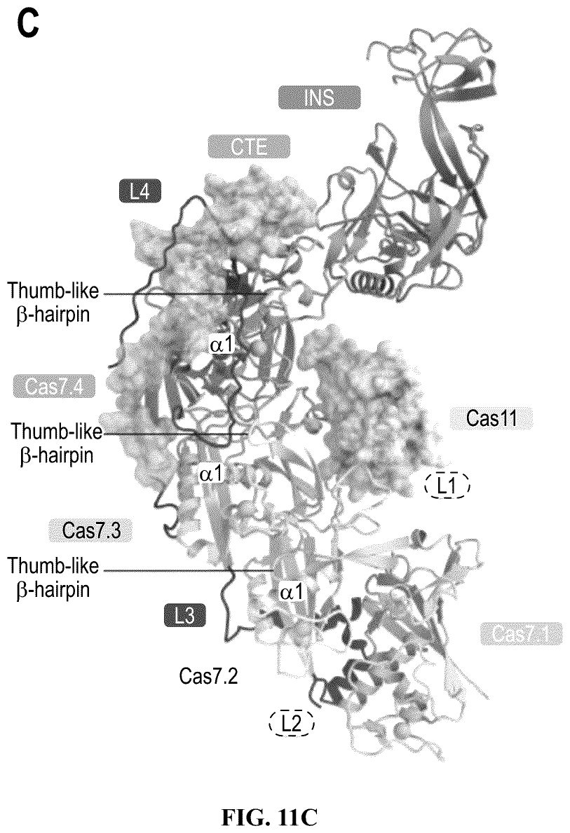

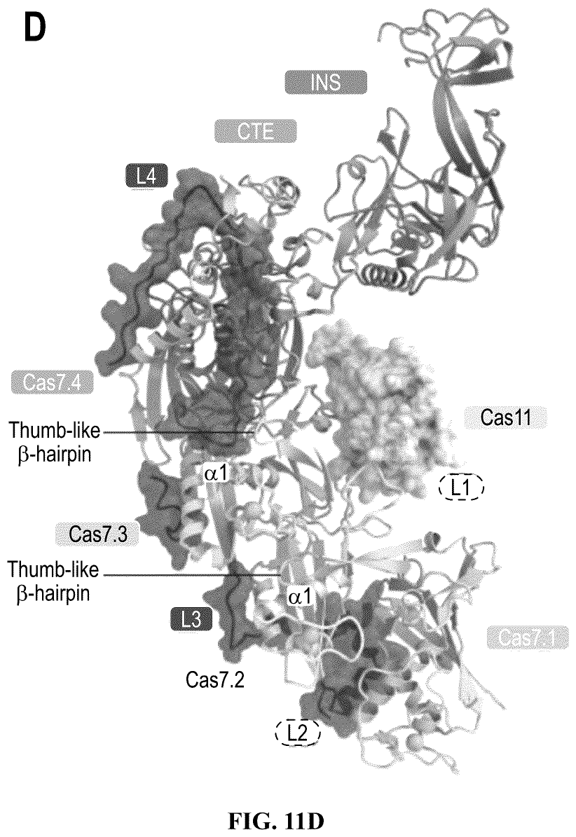

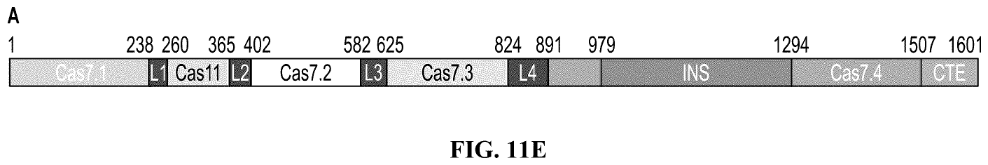

The domain interfaces of Cas7-11 were assessed. Cas7.1-Cas7.4 form a central filament in the Cas7-11 structure (FIGS. 11A and 11B). Cas7.1-Cas7.3 commonly employ two interfaces for the interaction with their adjacent Cas7 domains (Cas7.2-Cas7.4), as in the Cas7 filaments (Csm3/Cmr4) in the type III-A/B effectors (Taylor et al. 2015; Osawa et al. 2015; You et al. 2019; and Jia et al. 2019, incorporated herewith in their entirety by reference). At the primary interface, the β1-α1 and α2-β4 regions of a Cas7 domain interact with the β3 strand of the adjacent Cas7 domain. At the second interface, the thumb-like β-hairpin (in the β2-β3 region) of a Cas7 domain contacts the α1 helix of the adjacent Cas7 domain (FIGS. 11A and 11B). In addition to the two interfaces, each Cas7 domain forms distinct interactions with their adjacent Cas7 domains, consistent with the structural variations among the Cas7.1-Cas7.4 domains. The thumb-like β-hairpins of Cas7.1 and Cas7.2 form additional contacts with the zinc-finger loops (in the α1-β2 region) of Cas7.2/Cas7.3 and Cas7.3/Cas7.4, respectively (FIGS. 11A and 11B). In addition, the thumb-like β-hairpins of Cas7.2 and Cas7.3 interact with Cas7.3 (the additional β-hairpin) and Cas7.4 (the zinc-finger loop and the β1-α1 and β2-β3 regions), respectively (FIGS. 11A and 11B). The L3 and L4 linkers also contribute to stabilizing the Cas7.1-Cas7.4 filament structure. The L3 linker reinforces the interface between Cas7.1 and Cas7.2, while the L4 linker adopts a V-shaped conformation and extensively interacts with Cas7.2, Cas7.3, Cas7.4, and CTE (FIGS. 11C and 11D). Cas11 mainly interacts with the β1-α1 regions of Cas7.2 and Cas7.3 (FIGS. 11A and 11B).

### Example 12

### Pre-crRNA Processing Mechanism

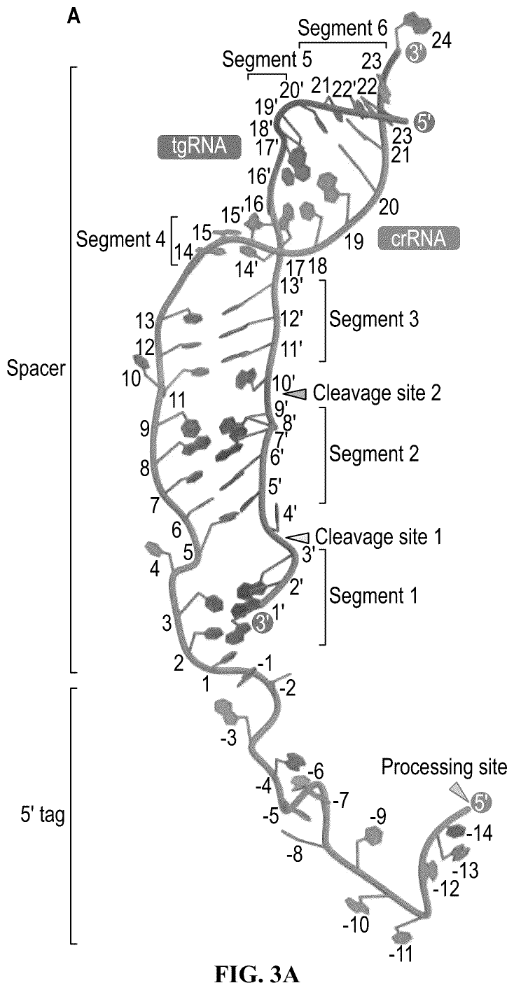

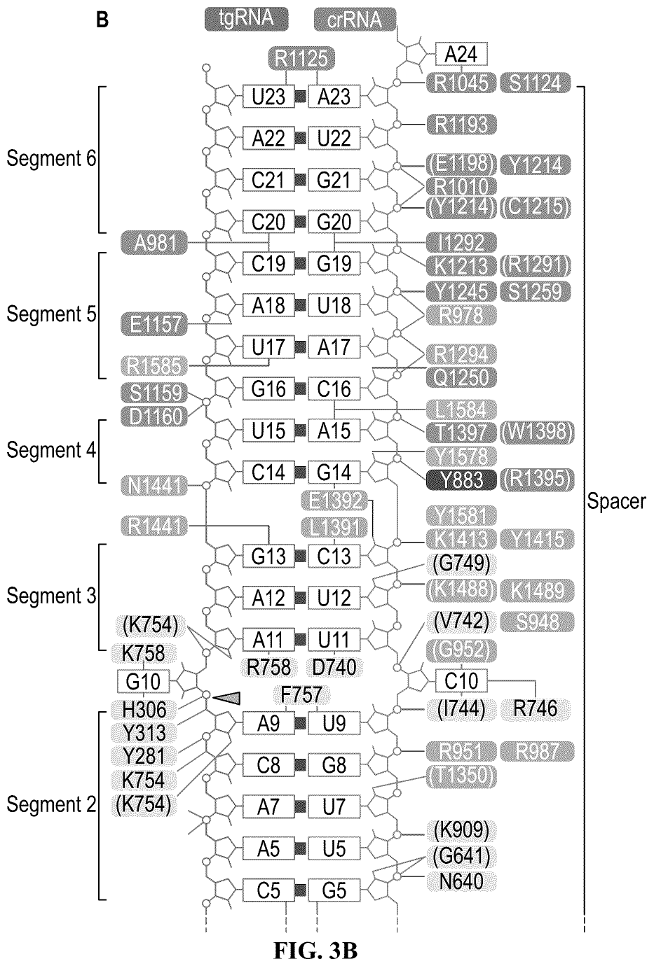

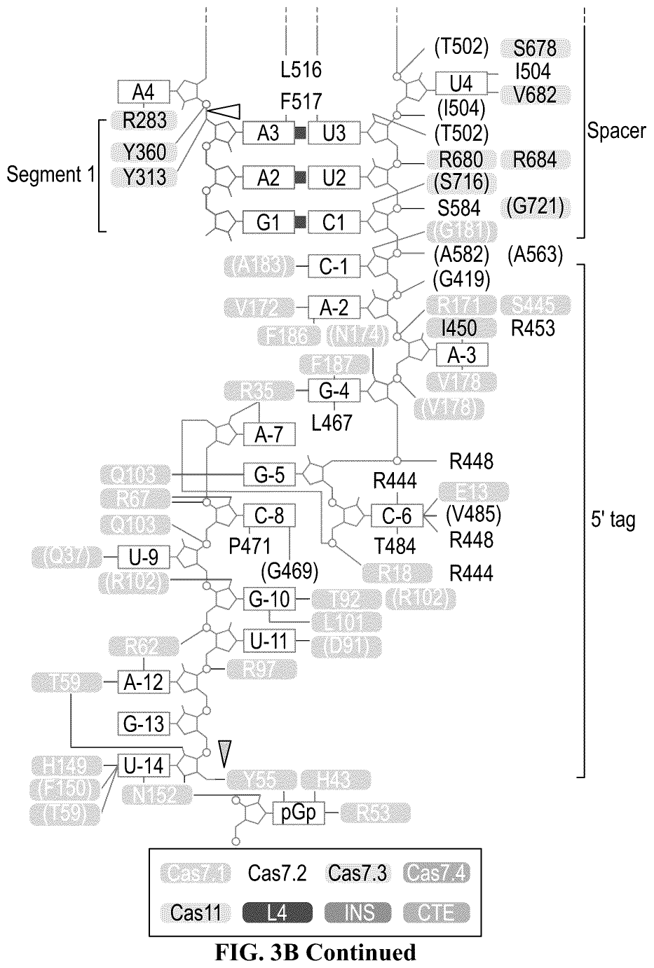

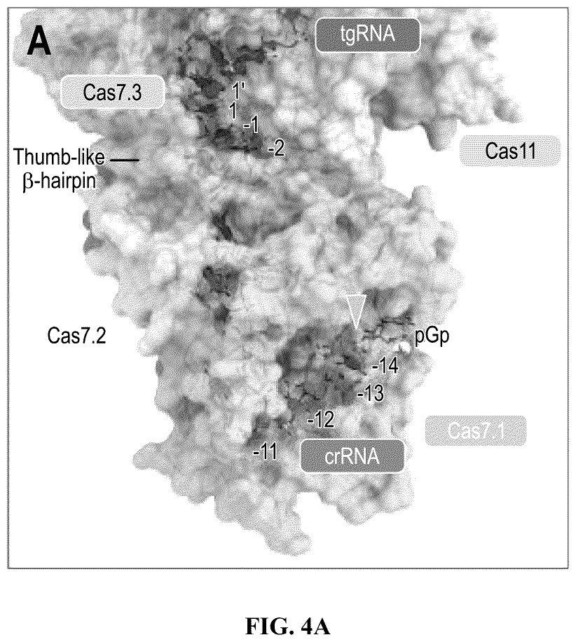

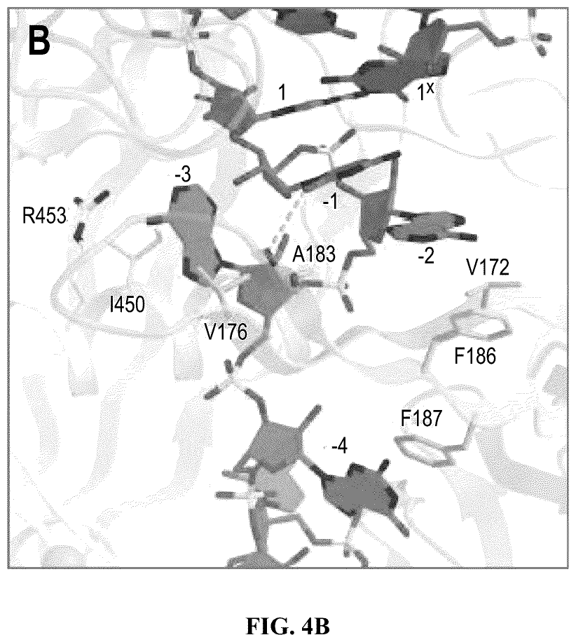

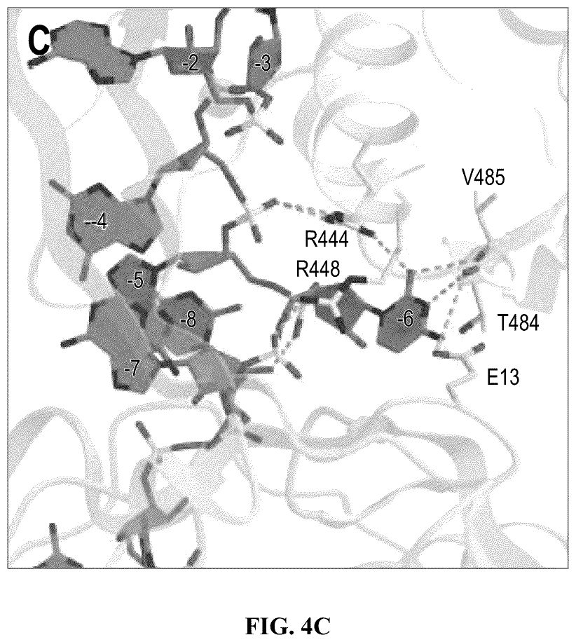

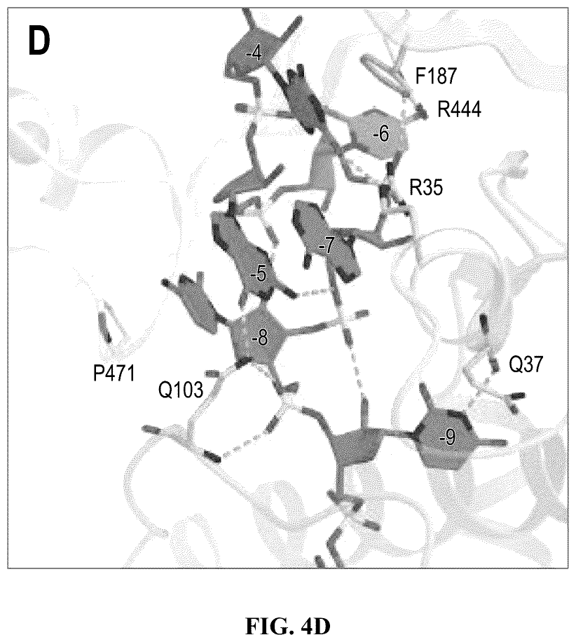

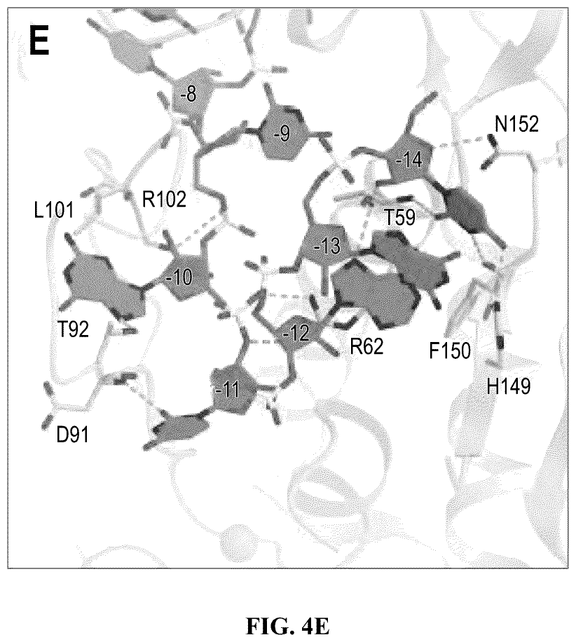

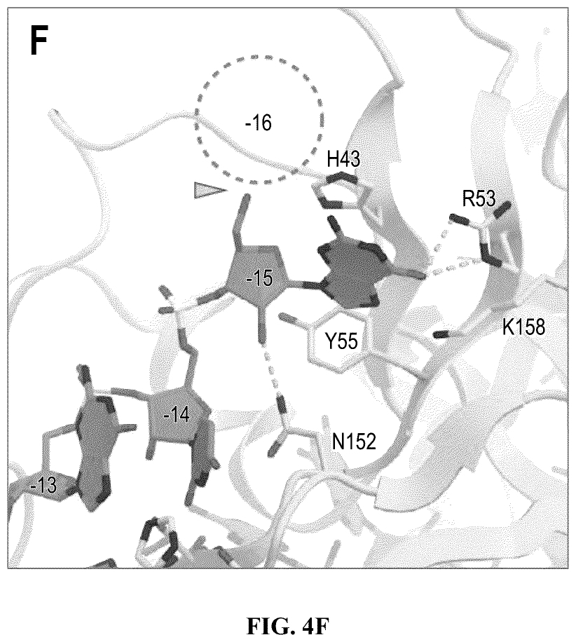

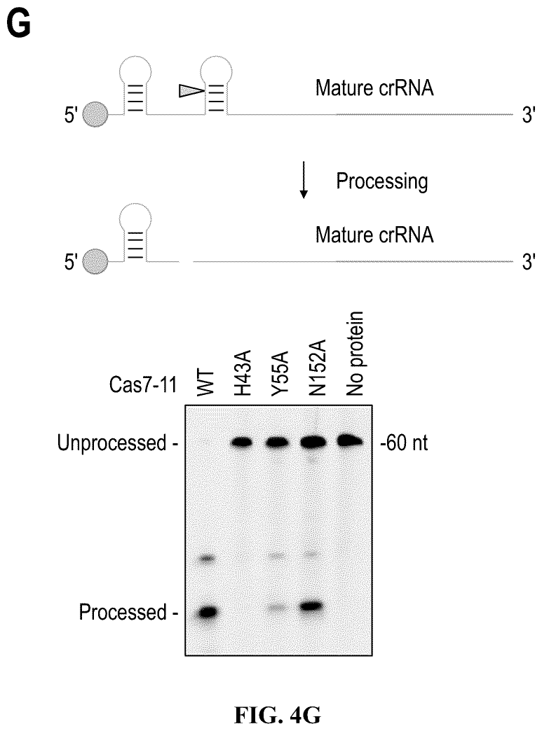

The Pre-crRNA processing mechanism was assessed. The 5′ tag region (U(−14)-C(−1)) of the crRNA adopts a single-stranded conformation and is extensively recognized by Cas7.1 and Cas7.2 (FIGS. 3A, 3B, 4A, and 12). The thumb-like β-hairpin of Cas7.1 intercalates between A(−2) and G(−4), resulting in the base flipping of A(−3) (FIG. 4B). The nucleobases of A(−2) and G(−4) stack with F186N172 and F187 in the β-hairpin, respectively, while the nucleobases of C(−1) and G(−4)hydrogen bond with A183 and R35, respectively. A(−3) stacks with V176, I450 and R453 in Cas7.2. C(−6) is also flipped out, and A(−7), G(−5), and C(−8) form a triple stack, which is sandwiched by R35 and P471 (FIGS. 4C and 4D). The flipped-out C(−6) forms a stacking interaction with R444 and T484, and multiple hydrogen bonds with E13, R448, and V485 (FIG. 4C), consistent with a previous finding that the mutation of C(−6) completely inhibited target RNA cleavage (Ozcan et al. 2021, incorporated herewith in its entirety by reference). G(−5) adopts the syn conformation, and hydrogen bonds with Q103 and the A(−7) backbone phosphate (FIG. 4D). The nucleobases of U(−9), G(−10), U(−11), and A(−12) hydrogen bond with Q37, T92/R102, D91, and T59, respectively (FIG. 4E). U(−14), the first nucleotide of the 5′ tag, forms hydrogen-bonding and stacking interactions with T59/H149/F150 and N152, respectively (FIG. 4E). In addition to these base-specific contacts, the 5′ tag region forms sequence-independent backbone interactions with Cas7.1 and Cas7.2 (FIG. 3B). The 14-nt 5′ tag sequence is highly conserved among Cas7-11 orthologs (Ozcan et al. 2021; and van Beljouw et al. 2021, incorporated herewith in their entirety by reference), therefore the 5′ tag regions may be recognized by Cas7-11 orthologs in a similar manner.

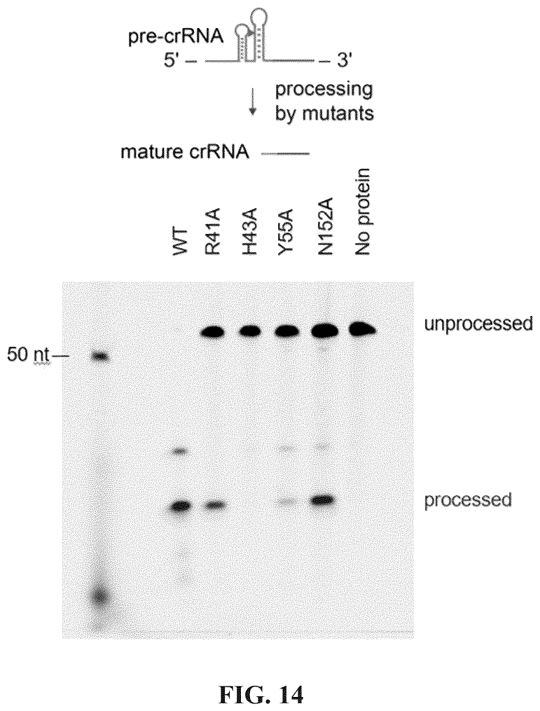

D. ishimotonii Cas7-11 cleaves pre-crRNAs between U(−15) and U(−14) of the direct repeat sequence in a metal-independent manner, to produce mature crRNAs with a 14-nt 5′ tag sequence (Ozcan et al. 2021, incorporated herewith in its entirety by reference). Metal-independent RNA hydrolysis via acid-base catalysis yields a 3′ product with a 5′-hydroxy group and a 5′ product with a 2′,3′-cyclic phosphate group, which is then converted to a 3′ phosphate group (Yang 2011, incorporated herewith in its entirety by reference). As the crRNA used for the cryoEM analysis contains G(−15), rather than U(−15), for an in vitro transcription reaction, a density corresponding to a guanosine-3′,5′-diphosphate (pGp) adjacent to U(−14), which contains a 5′-hydroxy group was observed (FIG. 4F). These structural observations indicates that, during the complex reconstitution, Cas7-11 cleaves the phosphodiester bond between G(−15) and U(−14) of the crRNA via an acid-base catalytic mechanism. The pGp molecule is surrounded by residues R41, H43, R53, Y55, N152, and K158 in the Cas7.1 domain (FIG. 4F), and H43, Y55, and N152 are highly conserved in the Cas7-11 orthologs (FIGS. 10A-10C). Thus, the H43A, Y55A, and N152A Cas7-11 mutants were prepared and their pre-crRNA processing and target RNA cleavage activity in vitro were tested (FIG. 14). It was observed that the Y55A and N152A mutants exhibit reduced pre-crRNA processing activities (FIG. 4G), confirming the functional importance of Y55 and H152 in the pre-crRNA processing. Notably, the H43A mutant lacks the processing activity (FIG. 4G), indicating that H43 is critical for the pre-crRNA processing. Thus, H43 may serve as a general base and deprotonates the 2′-hydroxy group of U(−15), which then nucleophilically attacks on the scissile phosphate between U(−15) and U(−14). These mutants were capable of target RNA cleavage (FIG. 4H). Therefore, Cas7-11 processes its pre-crRNAs in the Cas7.1 domain via an acid-base catalytic mechanism.

### Example 13

### Target RNA Recognition Mechanism

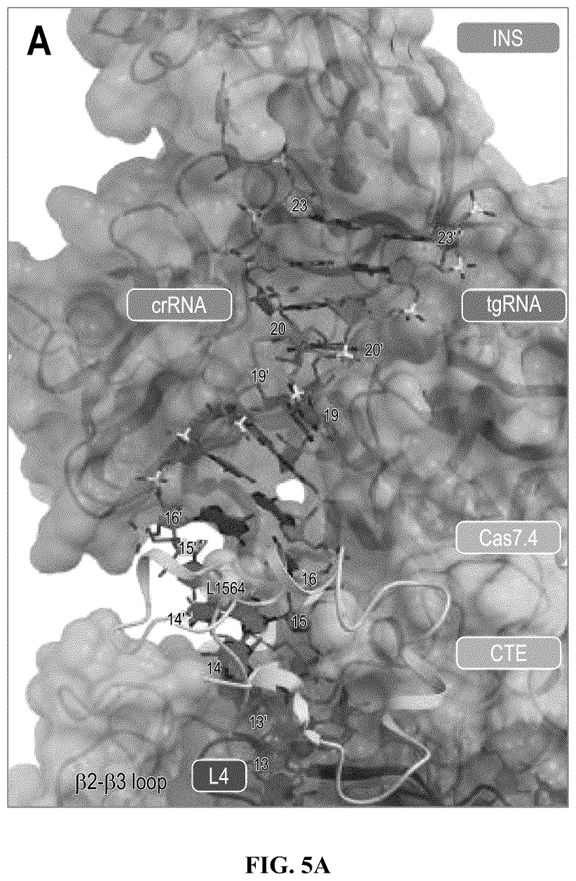

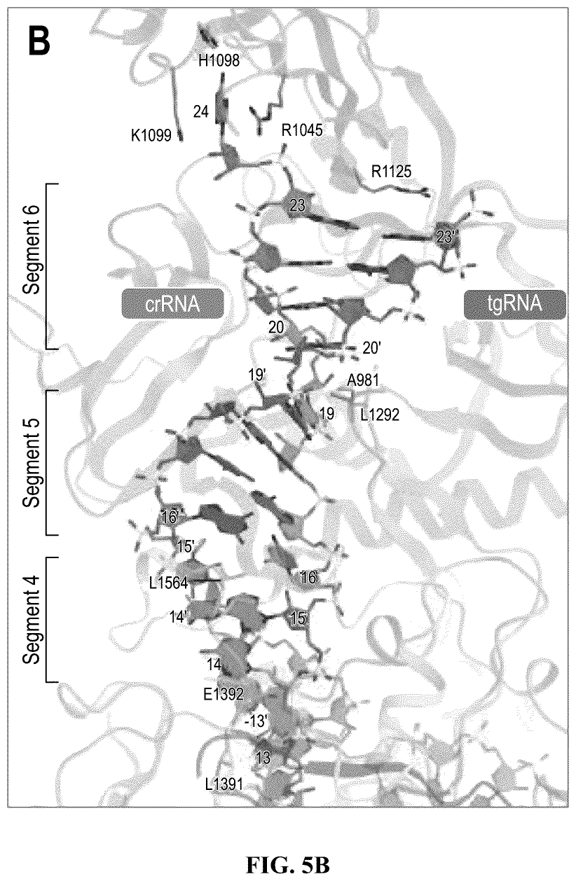

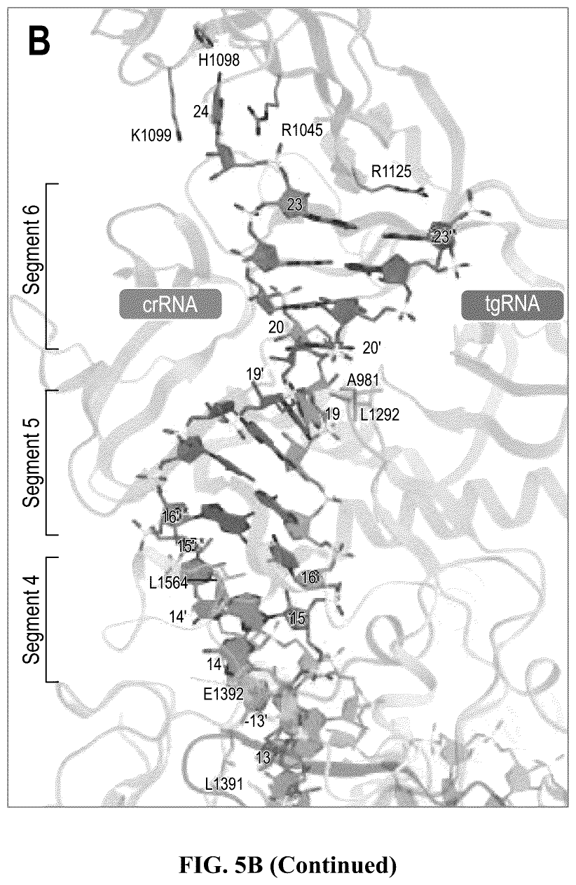

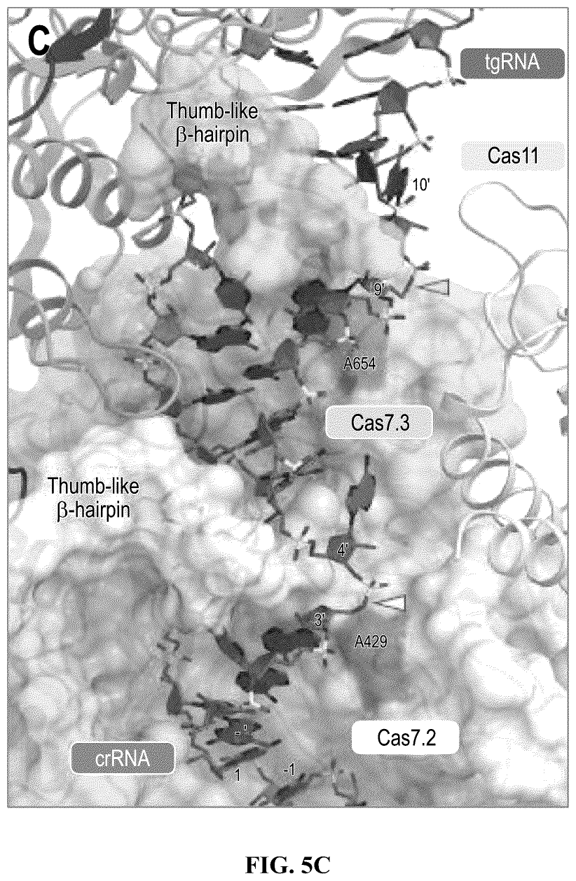

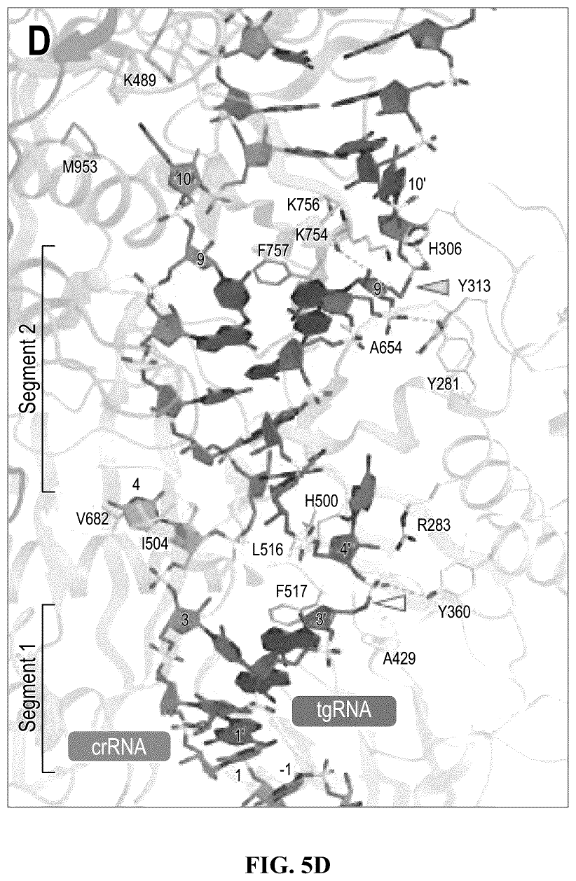

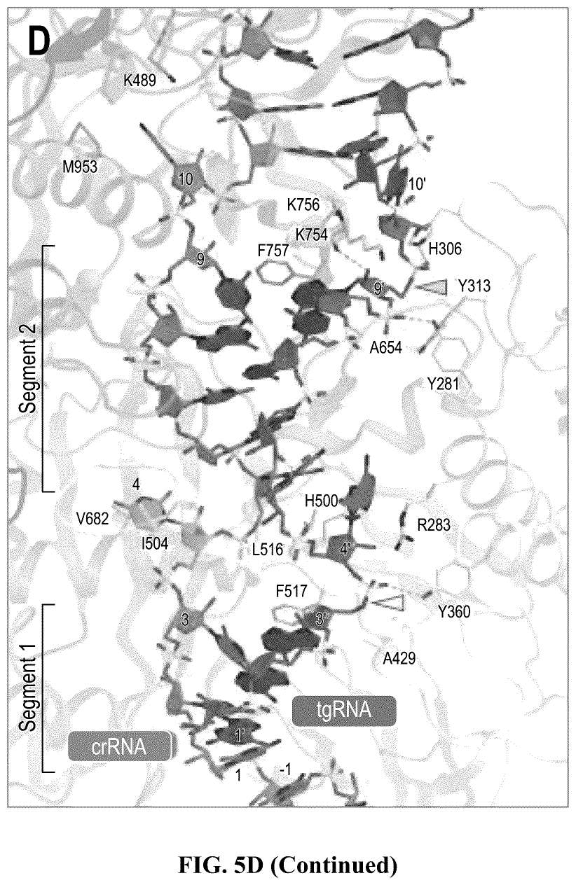

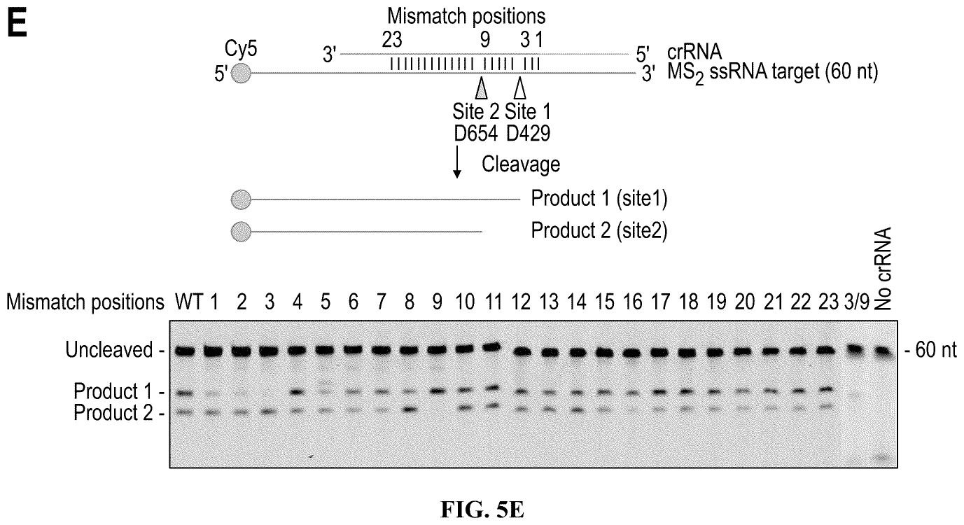

The RNA recognition mechanism was assessed. The spacer region (C1-A23, except for U4 and C10) of the crRNA base pairs with the target RNA to form the guide-target duplex, assisted by the INS domain (FIGS. 3A, 3B, 5A, 5B, and 12). The last base pair, A23-U23*, is capped by R1125 in the INS domain, while A24 is splayed out from the duplex and accommodated within a pocket formed by R1045, H1098, and K1099 in the INS domain (FIGS. 5A and 5B). U25 in the crRNA and U24*/A24* in the target RNA are disordered in the structure. These structural observations indicate that the 23-nt spacer sequence serves as a guide segment, consistent with a previous study showing that 22-25-nt, but not 19-nt, spacers support Cas7-11-mediated target RNA cleavage (Ozcan et al. 2021, incorporated herewith in its entirety by reference).

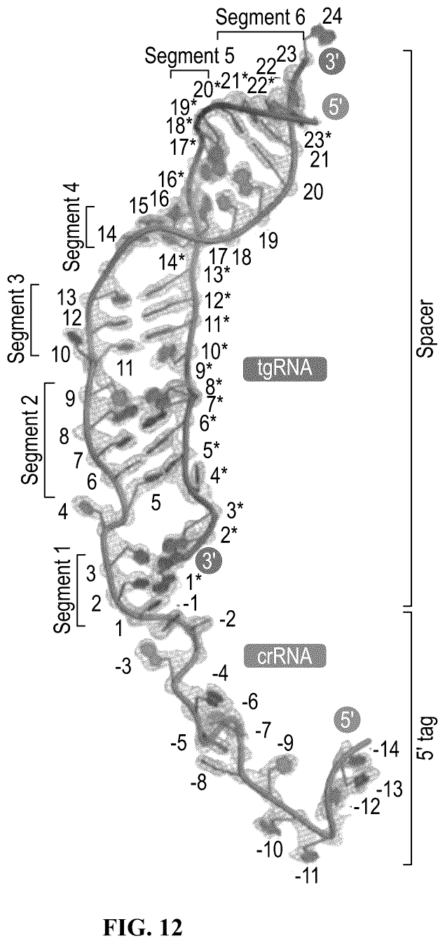

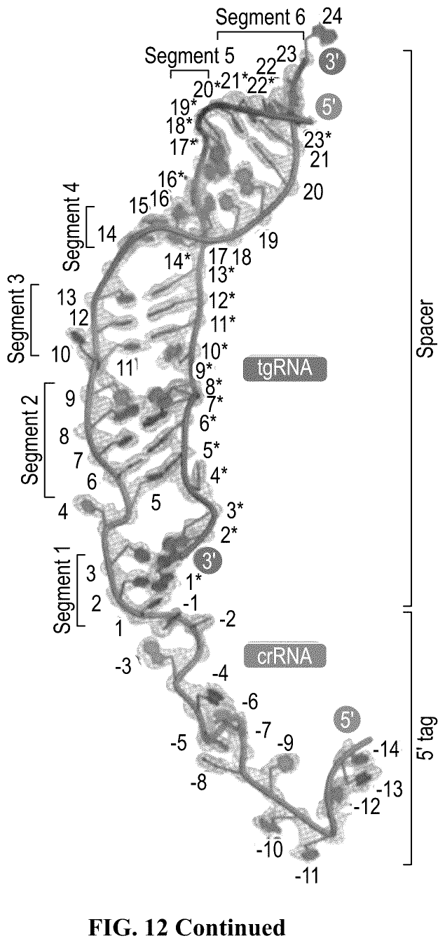

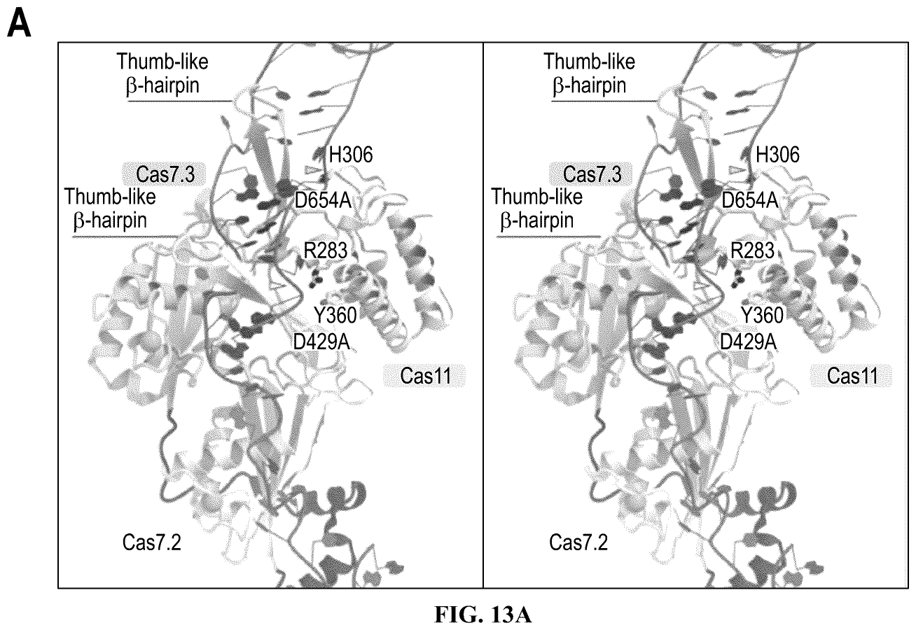

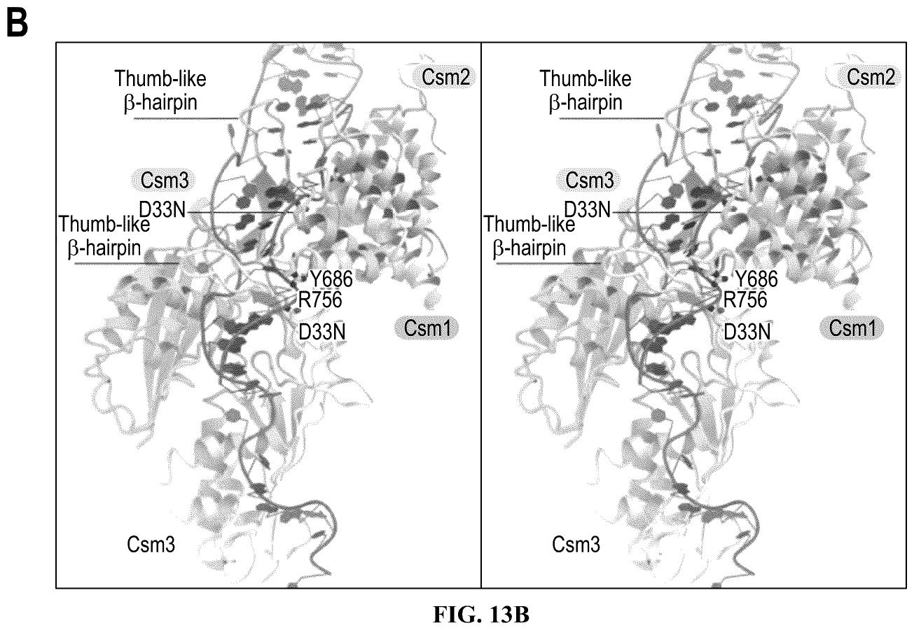

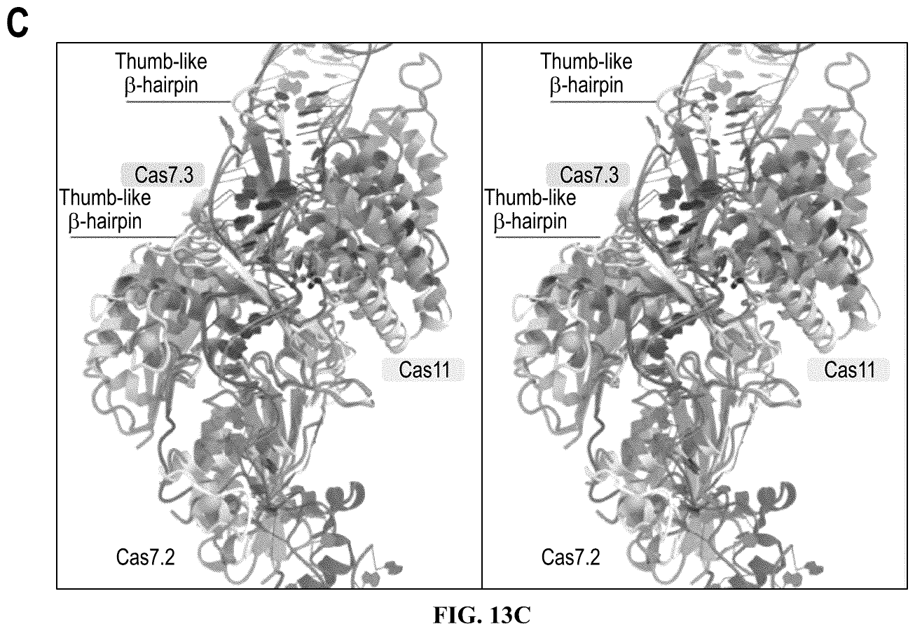

The 23-bp guide-target duplex consists of six segments (segments 1-6), consisting of successive base pairs, with the flipped-out nucleotides at fourth and tenth positions and kinks at the 13th-14th, 15th-16th, and 19th-20th base pairs (FIGS. 3A and 12). The thumb-like β-hairpins in Cas7.2 and Cas7.3 intercalate within the guide-target duplex, flipping the fourth and tenth nucleotides, respectively (FIGS. 5C and 5D). Accordingly, the segments 1-3 (C1-G1*-C13-G13*) adopt an underwound ribbon-like conformation resembling a ladder, rather than a double helix, similar to the guide-target duplexes in the type III-A/B effector complexes (Taylor et al. 2015; Osawa et al. 2015; You et al. 2019; and Jia et al. 2019, incorporated herewith in their entirety by reference)(FIGS. 5C, 5D, and 13A-13C). The U3-A3* and G5-C5* base pairs stack with F517 and L516 in the β-hairpin in Cas7.2, respectively, while the U9-A9* and U11-A11* base pairs stack with F757 and K756 in the β-hairpin in Cas7.3, respectively (FIG. 5D). The flipped-out U4 and C10 nucleobases are sandwiched by I504/V682 of Cas7.3 and M953/K1489 of Cas7.4, respectively. Appropriate for their involvement in recognition, three of these residues (I504, M953, and K1489) are conserved in both the Cas7-11 and Cas7×3 families, with V682 conserved within the Cas7-11 clade. The β2-β3 loop of Cas7.4 (equivalent to the thumb-like β-hairpins of Cas7.2 and Cas7.3) penetrates between the C13-G13* and G14-C14* base pairs in the guide-target duplex, resulting in a kink between the segments 3 and 4 (FIGS. 5A and 5B). The C13-G13* and G14-C14* base pairs interact with L1391 and E1392 in the β2-β3 loop of Cas7.4, respectively. L1564 in CTE and A981/I1292 in INS are wedged into the 15th-16th and 19th-20th base pairs, thereby forming a kink between the segments 4 and 5, and 5 and 6, respectively (FIG. 5B). Moreover, the sugar-phosphate backbone of the crRNA spacer region is extensively recognized by the Cas7.2-Cas7.4, INS, and CTE domains (FIG. 3B). These structural observations agree with conservation across these nuclease families and explain the mechanism of crRNA-guided target RNA recognition by Cas7-11.

### Example 14

### Target RNA Cleavage Mechanism

The RNA cleave mechanism was assessed D. ishimotonii Cas7-11 cleaves the target RNA with conserved aspartate residues (D429 and D654), generating two cleavage sites separated by 5-6 nt near the 3′ end of the spacer-complementary region, although the precise cleavage sites were not determined (Ozcan et al. 2021, incorporated herewith in its entirety by reference). As for the Cas7-11 (D429A/D654A)structure, A429 in Cas7.2 and A654 in Cas7.3 are located close to the phosphodiester bonds between A3* and A4* and between U9* and C10* in the target RNA, respectively (FIGS. 5C and 5D), appropriate for their role as the catalytic residues for target RNA cleavage. In addition, the backbone phosphate groups between A3* and A4* and between U9* and C10* are recognized by Y360 and H306 in Cas11, respectively (FIG. 5D). The configuration of D429 and D654 of Cas7-11 relative to the target RNA is similar to that of the catalytic aspartate residues (D33 in Csm3) of the type III-A effector complex (FIGS. 13A-13C). These structural observations indicates that D. ishimotonii Cas7-11 cleaves a target RNA between the third and fourth nucleotides (site 1) and between the ninth and tenth nucleotides (site 2), using D429 in Cas7.2 and D654 in Cas7.3, respectively, consistent with Scalindua brodae Cas7-11 (Sb-gRAMP) that cleaves a target RNA at these two positions (van Beljouw et al. 2021, incorporated herewith in its entirety by reference).

In vitro cleavage activities of Cas7-11 against a 5′-fluorescently labelled, 60-nt ssRNA target, using crRNAs with mismatched spacers (mm1-23) were measured to validate the structural observations. Mm4 and mm10 were observed to do not affect the target RNA cleavage (FIG. 5E), consistent with the base flipping at positions 4 and 10 in the Cas7-11 structure. In contrast, mm3 and mm9 was observed to abolish the cleavage at the sites 1 and 2 on the target RNA (FIG. 5E), respectively, indicating that base pairing at positions 3 and 9 are critical for the RNA target cleavage at the sites 1 and 2, respectively. Accordingly, double mismatch (mm3/9) abolishes the target RNA cleavage (FIG. 5E). While mm1 and mm2 attenuates the cleavage at the site 1 on the target RNA, mismatches at the other positions (mm5-8 and mm11-23) do not substantially affect the RNA cleavage (FIG. 5E). Overall, the structural and functional data shows that Cas7-11 cleaves ssRNA targets after third and ninth nucleotides, counting from the 3′ end of the spacer-complementary region, by the Cas7.2 and Cas7.3 domains, respectively.

### Example 15

### Structure-guided Engineering of Compact Cas7-11 Variants

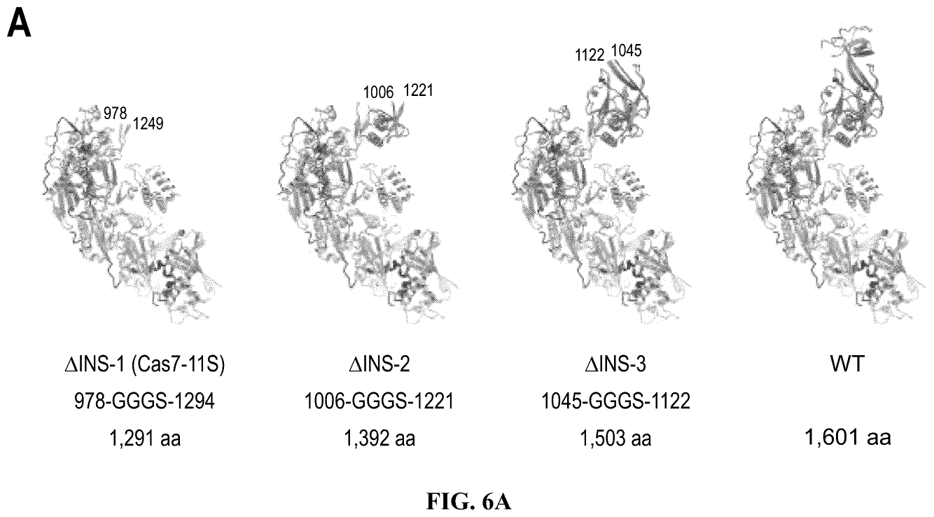

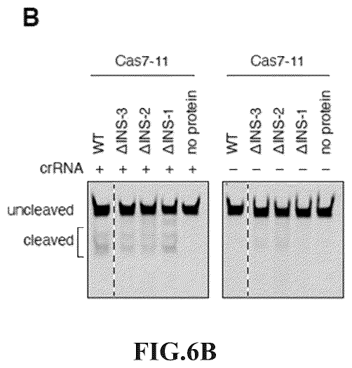

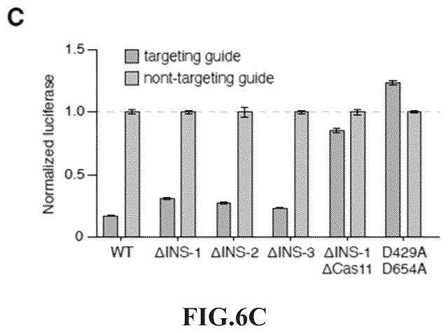

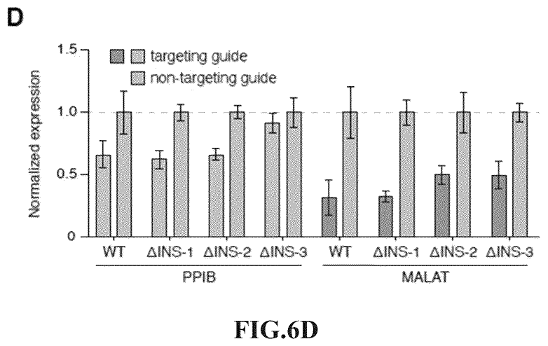

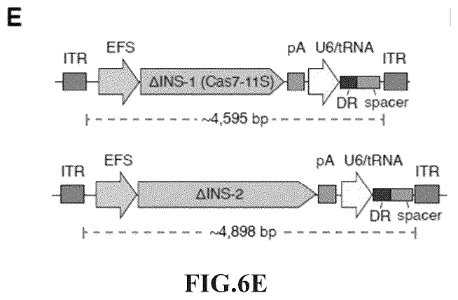

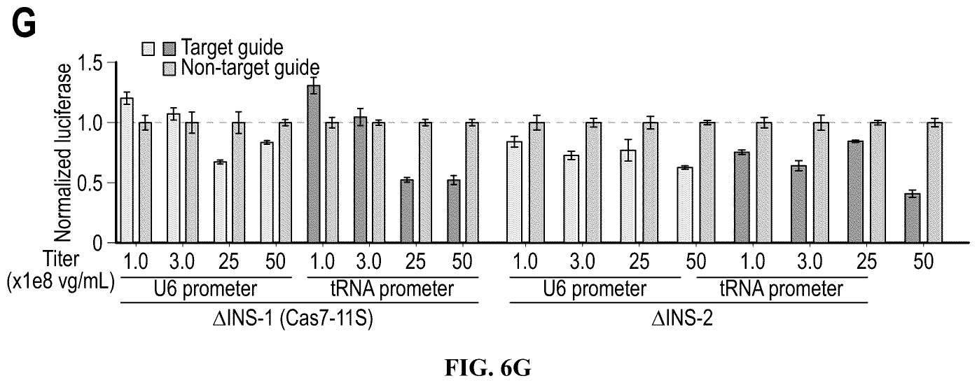

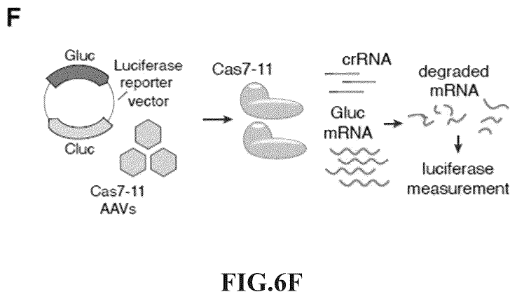

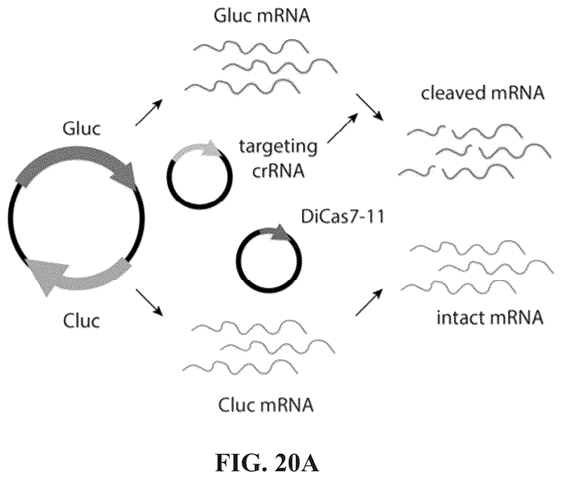

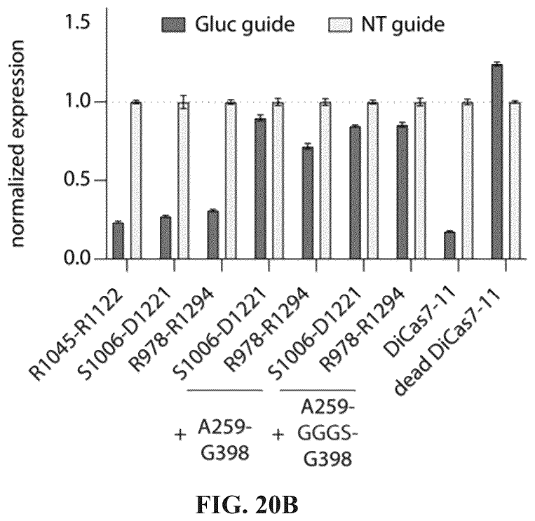

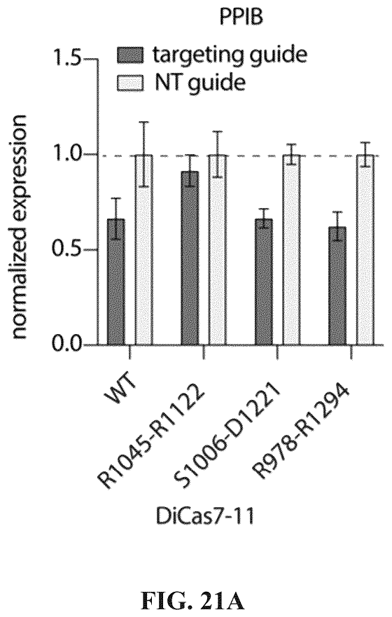

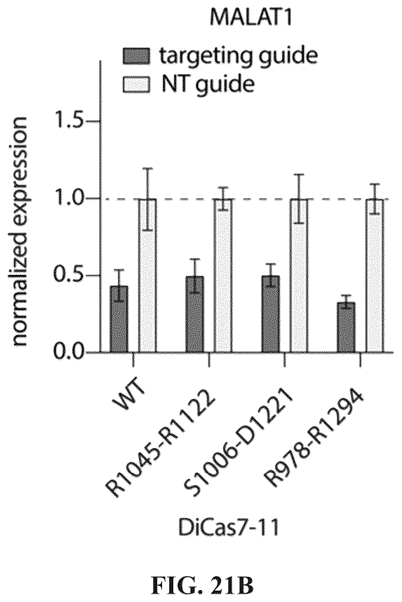

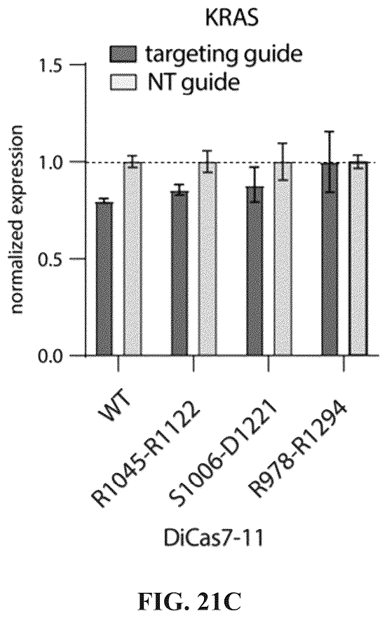

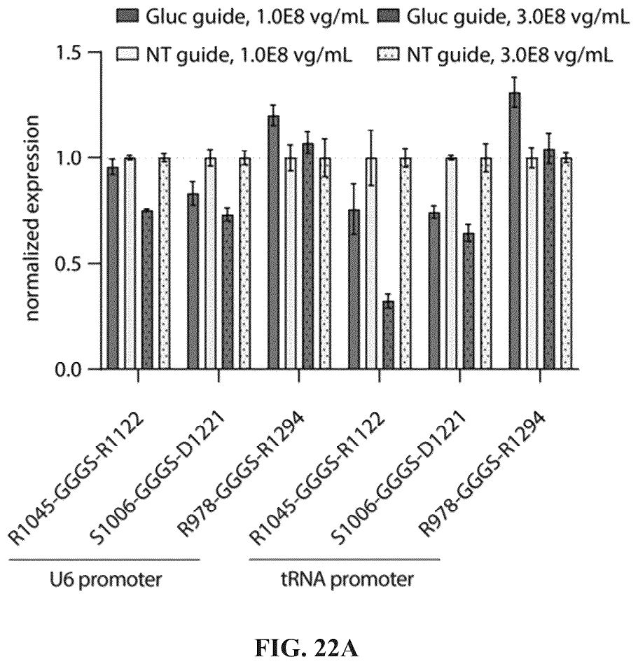

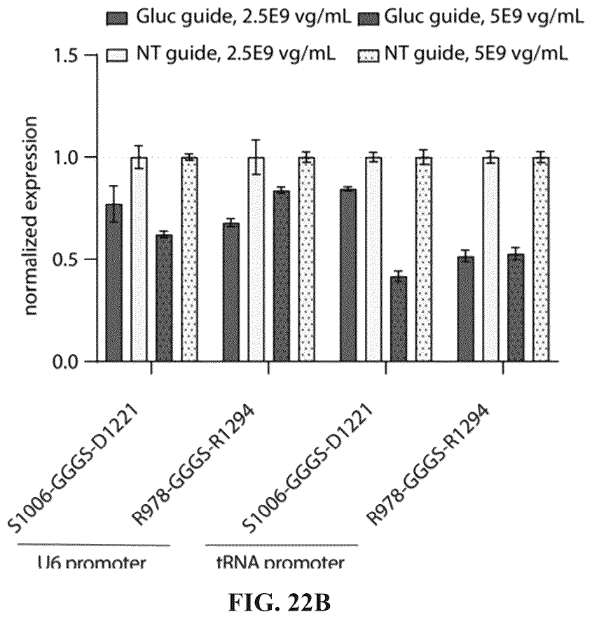

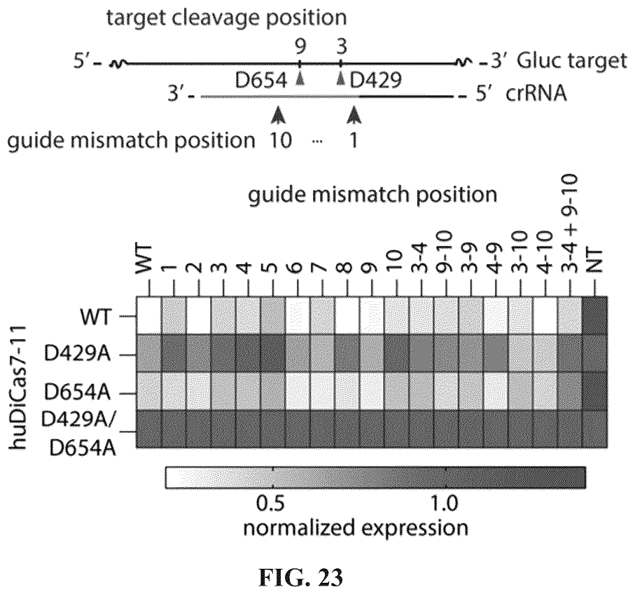

The structure-guided engineering of compact Cas7-11 variants was assessed. Three Cas7-11 deletion variants, ΔINS-1 (1,219 aa), ΔINS-2 (1,392 aa), and ΔINS-3 (1,530 aa), in which residues 979-1293, 1007-1220, and 1046-1121 are deleted and replaced with a GGGS linker, respectively were engineered (FIG. 6A). Their target RNA cleavage activities in vitro were measured, and it was found that the truncated Cas7-11 variants exhibit the same level of in vitro cleavage activity on the target RNA as compared to the full-length Cas7-11 (FIG. 6B). Using a dual-luciferase assay (Özcan et al. 2021, incorporated herewith in its entirety by reference), these truncated ΔINS Cas7-11 variants were demonstrated to preserve cleavage activity as compared to the wild-type (WT) Cas7-11 in mammalian cells (FIG. 6C). In contrast, the ΔCas11 variant (A259-GGGS-G398) greatly decreases Gluc transcript knockdown (FIG. 6C), indicating the importance of the Cas11 domain for target RNA cleavage. Both the WT Cas7-11 and ΔINS Cas7-11 variants also effectively knocks down endogenous mRNA targets (PPIB and MALAT) with comparable efficiency (FIG. 6D). Given its activity in mammalian cells and value as a tool for in vivo RNA-targeting, the smallest functional Cas7-11 truncation, ΔINS-1, was named as Cas7-11S. The reduced size of Cas7-11S and the ΔINS variants compared to WT Cas7-11 (1,601 aa) enables packaging along with a guide RNA cassette into single AAV viral vectors for delivery (FIG. 6E), with crRNA expression driven by either U6 or tRNA promoters. Testing Cas7-11S and ΔINS2 in HEK293FT cells via AAV8 delivery (FIG. 6F), both variants were found to be able to effectively knock down Gluc mRNA, with higher knockdown efficiencies at higher titers and with tRNA-driven crRNA expression (FIGS. 6G and 20-23).

### Example 16

### Cas7-11 Function and Mechanism

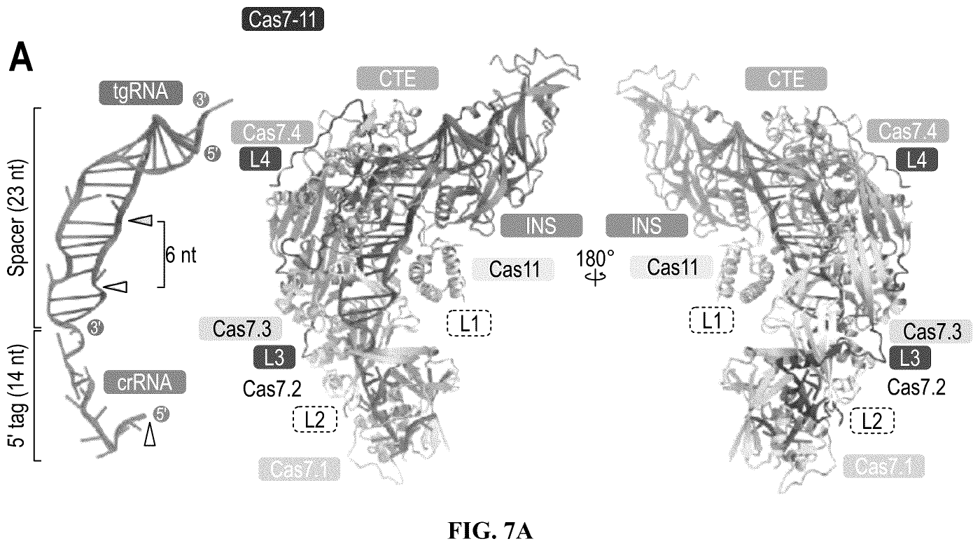

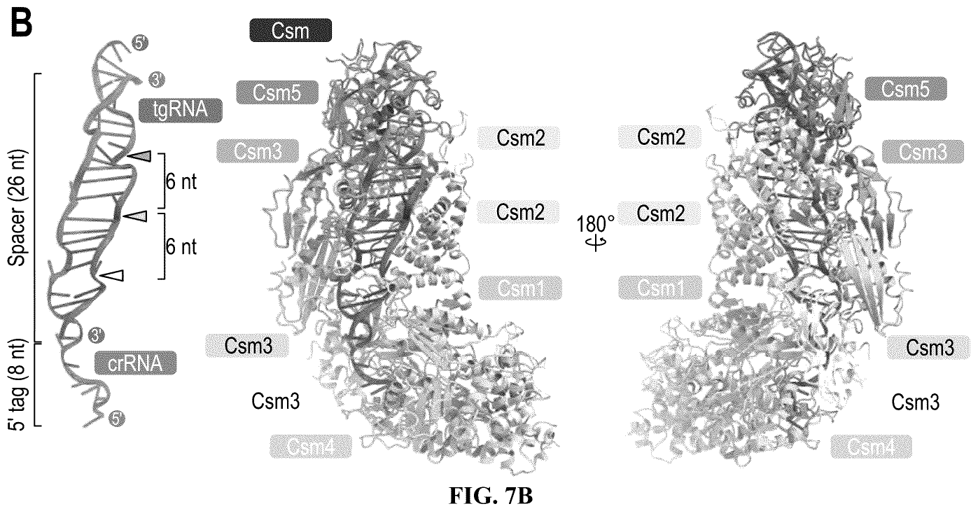

The function and mechanism of Cas7-11 were assessed. The structure of Cas7-11 in complex with its mature crRNA and target RNA was observed (FIGS. 7A-7B). The structure observed at 2.5-Å reveals that the four repetitive Cas7 domains and Cas11 are interwoven via four interdomain linkers (FIG. 7A). These linkers enable a multi-component type III system to emerge as a single-effector with all necessary domains encoded within the domains of a single protein and oriented correctly. The L4 linker is unique as it weaves through the protein from Cas7.3 around the CTE domain and to Cas7.4 with twists that allow these domains to fit together. The structure further reveals that Cas7.1 recognizes and binds the mature DR tag of the crRNA and is also containing the active site for pre-crRNA processing via an acid-base catalytic mechanism. As processing in type III complexes is typically carried out by the enzyme Cas6, this evolved function of type III-E effectors is unique and bears little resemblance to Cas6, allowing for Cas7-11 to function like other Class 2 systems that have incorporated pre-crRNA processing like Cas12 (e.g., (Swarts, van der Oost, and Jinek 2017), incorporated herewith in its entirety for reference) and Cas13 (e.g., East-Seletsky et al. 2016, incorporated herewith in its entirety for reference). Moreover, the processing domain of Cas7-11 (Cas7.1) has no resemblance to those of Cas12 (WED) and Cas13 (Helical-1), highlighting the mechanistic diversity of pre-crRNA processing in CRISPR-Cas systems. Beyond processing, the structure also reveals how Cas7.2, Cas7.3, and Cas7.4 bind the guide-target duplex and that through specific base flipping, Cas7.2 and Cas7.3 carry out precise cleavage 6-nt apart on the target, as in other type III systems like Csm3. By flipping out the bases of the target, Cas7-11 is able to cleave between the 3rd and 4th and 9th and 10th nucleotides of the target by placing catalytic aspartate residues in the vicinity of the scissile phosphodiester bonds. As other type III complexes typically have more Cas7 and Cas11 subunits than the equivalent domains of Cas7-11, they also have more cleavage sites with some complexes cleaving up to 5 times (e.g., Mohanraju et al. 2016, incorporated herewith in its entirety for reference) (FIG. 7B).

One skilled in the art will appreciate further features and advantages of the disclosure based on the above-described embodiments. Accordingly, the disclosure is not to be limited by what has been particularly shown and described, except as indicated by the appended claims.

### Example 17

### Single Mutant G-Luciferase Knockdown

Knockdown readout to measure cleavage activity on transcripts of luminescent Guassia luciferase protein by wildtype and mutant DiCas7-11 endonucleases was performed.

Sequences of mutant DiCas7-11 endonuclease coding sequences are shown below in Table 7 (short version of sequences showing in uppercase the residues that are responsible to encode mutations) and Table 8 (complete version of sequences).

TABLE 7
Residues responsible for mutation of DiCas7-11

| SEQ ID | Residues responsible for mutation of DiCas7-11 |
|---|---|
| NO/Description | endonuclease coding sequences |
| SEQ ID NO: 39 | gataataagaaaaccAAGtatacccgcctgcttg |
| E279K | |
| SEQ ID NO: 40 | gaaaaccgaaAAGacccgcctgc |
| Y280K | |
| SEQ ID NO: 41 | tgccaaaggatAAGgacgggaagga |
| H305K | |
| SEQ ID NO: 42 | gccaaaggatcatAAGgggaaggatgacc |
| D306K | |
| SEQ ID NO: 43 | gacgggaaggatAAGcattacctctggg |
| D310K | |
| SEQ ID NO: 44 | gaaggatgaccatAAGctctgggatatcgg |
| Y312K | |
| SEQ ID NO: 45 | ggtgaagcgctgAAGctcaagagtaaa |
| Y359K | |
| SEQ ID NO: 46 | cggttgcgAAGggtgccct |
| E512K | |
| SEQ ID NO: 47 | tggcggatgAAGgctgatcacc |
| D979K | |
| SEQ ID NO: 48 | cggatggatgctAAGcaccagaatgtg |
| D981K | |
| SEQ ID NO: 49 | atgtgctgcaaAAGtttctcccaggt |
| D988K | |
| SEQ ID NO: 50 | tagtgacaagaaagggAAGgtgatcaataactttca |
| D1163K | |
| SEQ ID NO: 51 | tcagggtctataacAAGaatcctcaagcagt |
| N1246K | |
| SEQ ID NO: 52 | tcattgttgAAGcggcctcgcc |
| E1391K | |
| SEQ ID NO: 53 | acaaagccctAAGaaccgcactgt |
| N1440K | |
| SEQ ID NO: 54 | gctgcgaaagaagAAGgatcctaatggga |
| D1567K | |
| SEQ ID NO: 55 | acgaagaactcaaaAAGggggaattcaagaa |
| D1580K | |
| SEQ ID NO: 56 | aataagaaaaccAGGtatacccgcctgc |
| E279R | |
| SEQ ID NO: 57 | aaaaccgaaAGGacccgcctgc |
| Y280R | |
| SEQ ID NO: 58 | gccaaaggatAGGgacgggaagg |
| H305R | |
| SEQ ID NO: 59 | ccaaaggatcatAGGgggaaggatgacc |
| D306R | |
| SEQ ID NO: 60 | acgggaaggatAGGcattacctctgg |
| D310R | |
| SEQ ID NO: 61 | aaggatgaccatAGGctctgggatatcg |
| Y312R | |
| SEQ ID NO: 62 | gtgaagcgctgAGGctcaagagtaa |
| Y359R | |
| SEQ ID NO: 63 | ggttgcgAGGggtgccct |
| E512R | |
| SEQ ID NO: 64 | tggcggatgAGGgctgatcac |
| D979R | |
| SEQ ID NO: 65 | ggatggatgctAGGcaccagaatgtg |
| D981R | |
| SEQ ID NO: 66 | tgtgctgcaaAGGtttctcccagg |
| D988R | |
| SEQ ID NO: 67 | agtgacaagaaagggAGGgtgatcaataacttt |
| D1163R | |
| SEQ ID NO: 68 | cagggtctataacAGGaatcctcaagcagt |
| N1246R | |
| SEQ ID NO: 69 | cattgttgAGGcggcctcgc |
| E1391R | |
| SEQ ID NO: 70 | acaaagccctAGGaaccgcactg |
| N1440R | |
| SEQ ID NO: 71 | ctgcgaaagaagAGGgatcctaatggga |
| D1567R | |
| SEQ ID NO: 72 | cgaagaactcaaaAGAggggaattcaaga |
| D1580R | |

TABLE 8

| SEQ ID | |
|---|---|
| NO/ | |
| Description | Mutant DiCas7-11 endonuclease coding sequences |
| SEQ | ATGACGACTACTATGAAGATTTCAATTGAATTCCTCGAGCCGTTTCGGATGACCAA |
| ID | ATGGCAGGAAAGCACTAGGCGAAACAAGAACAACAAGGAATTTGTTCGCGGCCAA |
| NO: 119 | GCTTTTGCAAGATGGCATAGAAATAAGAAGGATAACACAAAAGGGCGGCCTTACAT |
| E279K | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCAAGTATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 120 | ATGGCAGGAAAGCACTAGGCGAAACAAGAACAACAAGGAATTTGTTCGCGGCCAA |
| Y280K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAAAAGACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 121 | ATGGCAGGAAAGCACTAGGCGAAACAAGAACAACAAGGAATTTGTTCGCGGCCAA |
| H305K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATAAGGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 122 | ATGGCAGGAAAGCACTAGGCGAAACAAGAACAACAAGGAATTTGTTCGCGGCCAA |
| D306K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATAAGGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 123 | ATGGCAGGAAAGCACTAGGCGAAACAAGAACAACAAGGAATTTGTTCGCGGCCAA |
| D310K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATAAGCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 124 | ATGGCAGGAAAGCACTAGGCGAAACAAGAACAACAAGGAATTTGTTCGCGGCCAA |
| Y312K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATAAGCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 125 | ATGGCAGGAAAGCACTAGGCGAAACAAGAACAACAAGGAATTTGTTCGCGGCCAA |
| Y359K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGAAGCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 126 | ATGGCAGGAAAGCACTAGGCGAAACAAGAACAACAAGGAATTTGTTCGCGGCCAA |
| E512K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGAAGGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 127 | ATGGCAGGAAAGCACTAGGCGAAACAAGAACAACAAGGAATTTGTTCGCGGCCAA |
| D979K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | AAGGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 128 | ATGGCAGGAAAGCACTAGGCGAAACAAGAACAACAAGGAATTTGTTCGCGGCCAA |
| D981K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTAAGCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 129 | ATGGCAGGAAAGCACTAGGCGAAACAAGAACAACAAGGAATTTGTTCGCGGCCAA |
| D988K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAAAGTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 130 | ATGGCAGGAAAGCACTAGGCGAAACAAGAACAACAAGGAATTTGTTCGCGGCCAA |
| D1163K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGAAG |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 131 | ATGGCAGGAAAGCACTAGGCGAAACAAGAACAACAAGGAATTTGTTCGCGGCCAA |
| N1246K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAAGAATCCTCAAGCAGTGCCGGAGAGCGTATT |
| | TCAGTCTAGAGTTGCCCGGGAAAACGTGGAAAAGCTGAAGTCCGGAGATCTTGTG |
| | TATTTCAAACATAATGAAAAGTACGTAGAGGACATCGTCCCAGTGCGGATTTCCCG |
| | AACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 132 | ATGGCAGGAAAGCACTAGGCGAAACAAGAACAACAAGGAATTTGTTCGCGGCCAA |
| E1391K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGAAGCGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 133 | ATGGCAGGAAAGCACTAGGCGAAACAAGAACAACAAGGAATTTGTTCGCGGCCAA |
| N1440K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | AGAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 134 | ATGGCAGGAAAGCACTAGGCGAAACAAGAACAACAAGGAATTTGTTCGCGGCCAA |
| D1567K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGAAGGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 135 | ATGGCAGGAAAGCACTAGGCGAAACAAGAACAACAAGGAATTTGTTCGCGGCCAA |
| D1580K | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAAAGGGGGAATTCAAGAAAGAAG |
| | ATCGGCAGAAGAAGCTGACCACGCCGTGGACACCGTGGGCATAG |
| SEQ ID | ATGGCAGGAAAGCACTAGGCGAAACAAGAACAACAAGGAATTTGTTCGCGGCCAA |
| NO: 136 | ATGACGACTACTATGAAGATTTCAATTGAATTCCTCGAGCCGTTTCGGATGACCAA |
| E279R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCAGGTATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 137 | ATGGCAGGAAAGCACTAGGCGAAACAAGAACAACAAGGAATTTGTTCGCGGCCAA |
| Y280R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAAAGGACCCGCCTGCTTGCTGATGCTATTCGTTCACTCCGCCGCTCTAGTA |
| | AACTTGTCGCTGGCTTGCCAAAGGATCATGACGGGAAGGATGACCATTACCTCTG |
| | GGATATCGGCAAGAAGAAGAAAGACGAAAACTCTGTCACTATTAGGCAAATCCTTA |
| | CGACCTCAGCAGACACCAAGGAACTCAAGAATGCCGGAAAATGGAGAGAATTCTG |
| | CGAGAAGCTGGGTGAAGCGCTGTACCTCAAGAGTAAAGACATGAGTGGCGGCCT |
| | GAAAATTACTCGGAGAATACTGGGCGATGCTGAATTCCATGGAAAGCCCGATCGG |
| | CTTGAAAAGAGCCGGTCTGTGTCAATCGGATCTGTGTTGAAAGAAACTGTGGTATG |
| | CGGCGAACTGGTCGCTAAAACGCCCTTCTTCTTCGGAGCGATAGACGAAGATGCA |
| | AAACAAACCgacCTGCAAGTACTCCTCACTCCCGATAACAAGTATAGACTGCCAAGA |
| | AGCGCCGTGCGAGGTATACTCCGTCGAGATCTTCAAACCTATTTTGATAGCCCATG |
| | CAATGCTGAGTTGGGTGGACGGCCATGCATGTGTAAGACGTGTAGGATTATGAGA |
| | GGGATCACGGTGATGGATGCGCGCAGTGAGTACAATGCCCCGCCAGAAATAAGG |
| | CATCGTACCCGCATTAATCCCTTCACAGGCACGGTTGCGGAAGGTGCCCTGTTTA |
| | ATATGGAAGTAGCCCCCGAGGGGATTGTCTTTCCATTTCAACTCCGGTACCGGGG |
| | CTCTGAAGATGGGCTGCCCGATGCACTGAAAACGGTGTTGAAATGGTGGGCTGAG |
| | GGGCAGGCATTCATGAGTGGCGCTGCCTCAACCGGGAAGGGCCGATTCCGGATG |
| | GAAAATGCTAAATATGAAACGCTGGATCTGAGCGACGAGAATCAAAGGAATGACTA |
| | TCTTAAGAATTGGGGATGGCGTGACGAGAAGGGGCTCGAGGAACTGAAGAAACG |
| | ACTGAACTCAGGTCTGCCAGAGCCCGGTAATTATAGGGATCCAAAATGGCACGAG |
| | ATTAACGTTTCCATTGAGATGGCAAGCCCTTTTATTAATGGCGACCCAATCCGCGC |
| | AGCCGTGGACAAACGTGGTACAgatGTGGTTACCTTCGTTAAGTATAAAGCTGAAG |
| | GGGAAGAGGCGAAACCCGTATGTGCATACAAGGCCGAATCTTTTAGAGGGGTGAT |
| | CAGAAGTGCCGTGGCACGCATTCATATGGAAGATGGCGTCCCTTTGACTGAGTTG |
| | ACTCACAGTGACTGTGAATGTCTCCTGTGCCAAATCTTTGGAAGTGAGTATGAAGC |
| | CGGCAAAATAAGGTTTGAAGATCTCGTATTCGAAAGTGACCCGGAACCTGTGACCT |
| | TCGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 138 | ATGGCAGGAAAGCACTAGGCGAAACAAGAACAACAAGGAATTTGTTCGCGGCCAA |
| H305R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATAGGGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 139 | ATGGCAGGAAAGCACTAGGCGAAACAAGAACAACAAGGAATTTGTTCGCGGCCAA |
| D306R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATAGGGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 140 | ATGGCAGGAAAGCACTAGGCGAAACAAGAACAACAAGGAATTTGTTCGCGGCCAA |
| D310R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATAGGCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 141 | ATGGCAGGAAAGCACTAGGCGAAACAAGAACAACAAGGAATTTGTTCGCGGCCAA |
| Y312R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATAGGCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 142 | ATGGCAGGAAAGCACTAGGCGAAACAAGAACAACAAGGAATTTGTTCGCGGCCAA |
| Y359R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGAGGCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 143 | ATGGCAGGAAAGCACTAGGCGAAACAAGAACAACAAGGAATTTGTTCGCGGCCAA |
| E512R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGAGGGGTGCCCTGTTTAA |
| | TATGGAAGTAGCCCCCGAGGGGATTGTCTTTCCATTTCAACTCCGGTACCGGGGC |
| | TCTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ | ATGACGACTACTATGAAGATTTCAATTGAATTCCTCGAGCCGTTTCGGATGACCAA |
| ID NO: 144 | ATGGCAGGAAAGCACTAGGCGAAACAAGAACAACAAGGAATTTGTTCGCGGCCAA |
| D979R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | AGGGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 145 | ATGGCAGGAAAGCACTAGGCGAAACAAGAACAACAAGGAATTTGTTCGCGGCCAA |
| D981R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTAGGCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 146 | ATGGCAGGAAAGCACTAGGCGAAACAAGAACAACAAGGAATTTGTTCGCGGCCAA |
| D988R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAAGGTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 147 | ATGGCAGGAAAGCACTAGGCGAAACAAGAACAACAAGGAATTTGTTCGCGGCCAA |
| D1163R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGAGG |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 148 | ATGGCAGGAAAGCACTAGGCGAAACAAGAACAACAAGGAATTTGTTCGCGGCCAA |
| N1246R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAGGAATCCTCAAGCAGTGCCGGAGAGCGTATT |
| | TCAGTCTAGAGTTGCCCGGGAAAACGTGGAAAAGCTGAAGTCCGGAGATCTTGTG |
| | TATTTCAAACATAATGAAAAGTACGTAGAGGACATCGTCCCAGTGCGGATTTCCCG |
| | AACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 149 | ATGGCAGGAAAGCACTAGGCGAAACAAGAACAACAAGGAATTTGTTCGCGGCCAA |
| E1391R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGAGGCGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 150 | ATGGCAGGAAAGCACTAGGCGAAACAAGAACAACAAGGAATTTGTTCGCGGCCAA |
| N1440R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | GGAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ATGACGACTACTATGAAGATTTCAATTGAATTCCTCGAGCCGTTTCGGATGACCAA |
| NO: 151 | ATGGCAGGAAAGCACTAGGCGAAACAAGAACAACAAGGAATTTGTTCGCGGCCAA |
| D1567R | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACCGAATATACCCGCCTGCTTGCTGATGCTATTCGTTCACTCCGCCGCTCTAGTAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGAGGGAT |
| | CCTAATGGGAATAGCGGTTACGAAGAACTCAAAGACGGGGAATTCAAGAAAGAAG |
| | ATCGGCAGAAGAAGCTGACCACGCCGTGGACACCGTGGGCATAG |
| SEQ ID | ACCGAATATACCCGCCTGCTTGCTGATGCTATTCGTTCACTCCGCCGCTCTAGTAA |
| NO: 152 | ATGACGACTACTATGAAGATTTCAATTGAATTCCTCGAGCCGTTTCGGATGACCAA |
| D1580R | ATGGCAGGAAAGCACTAGGCGAAACAAGAACAACAAGGAATTTGTTCGCGGCCAA |
| | GCTTTTGCAAGATGGCATAGAAATAAGAAGGATAACACAAAAGGGCGGCCTTACAT |
| | AACTGGGACATTGCTGCGGTCCGCAGTAATTAGAAGCGCGGAAAACCTGCTGACA |
| | TTGAGTGATGGGAAAATTAGCGAAAAGACATGTTGTCCTGGGAAATTTGATACGGA |
| | AGATAAAGACAGGTTGCTGCAATTGAGGCAGCGGTCCACCCTTCGTTGGACCGAT |
| | AAGAACCCCTGTCCAGATAACGCTGAAACTTACTGCCCGTTTTGCGAACTTCTCGG |
| | GAGATCCGGTAACGATGGAAAGAAGGCTGAGAAGAAGGATTGGCGCTTTCGTATT |
| | CATTTTGGCAATCTTTCCCTTCCAGGGAAGCCCGATTTCGACGGGCCAAAAGCTAT |
| | AGGCAGCCAACGGGTACTTAACAGGGTCGATTTCAAATCCGGTAAGGCCCATGAC |
| | TTCTTTAAGGCTTACGAAGTCGACCATACTAGGTTTCCCCGCTTCGAAGGGGAGAT |
| | TACCATAGATAATAAAGTCAGCGCTGAAGCCAGGAAACTGCTCTGTGATTCTCTCA |
| | AGTTTACGGATCGGCTGTGTGGAGCTCTGTGCGTAATCAGGTTCGATGAATATACA |
| | CCAGCAGCCGATAGTGGGAAACAAACCGAAAATGTCCAAGCAGAACCGAATGCTA |
| | ATCTCGCTGAAAAGACCGCAGAGCAAATTATTAGCATTCTGGACGATAATAAGAAA |
| | ACTTGTCGCTGGCTTGCCAAAGGATCATGACGGGAAGGATGACCATTACCTCTGG |
| | GATATCGGCAAGAAGAAGAAAGACGAAAACTCTGTCACTATTAGGCAAATCCTTAC |
| | GACCTCAGCAGACACCAAGGAACTCAAGAATGCCGGAAAATGGAGAGAATTCTGC |
| | GAGAAGCTGGGTGAAGCGCTGTACCTCAAGAGTAAAGACATGAGTGGCGGCCTG |
| | AAAATTACTCGGAGAATACTGGGCGATGCTGAATTCCATGGAAAGCCCGATCGGC |
| | TTGAAAAGAGCCGGTCTGTGTCAATCGGATCTGTGTTGAAAGAAACTGTGGTATGC |
| | GGCGAACTGGTCGCTAAAACGCCCTTCTTCTTCGGAGCGATAGACGAAGATGCAA |
| | AACAAACCgacCTGCAAGTACTCCTCACTCCCGATAACAAGTATAGACTGCCAAGAA |
| | GCGCCGTGCGAGGTATACTCCGTCGAGATCTTCAAACCTATTTTGATAGCCCATGC |
| | AATGCTGAGTTGGGTGGACGGCCATGCATGTGTAAGACGTGTAGGATTATGAGAG |
| | GGATCACGGTGATGGATGCGCGCAGTGAGTACAATGCCCCGCCAGAAATAAGGC |
| | ATCGTACCCGCATTAATCCCTTCACAGGCACGGTTGCGGAAGGTGCCCTGTTTAAT |
| | ATGGAAGTAGCCCCCGAGGGGATTGTCTTTCCATTTCAACTCCGGTACCGGGGCT |
| | CTGAAGATGGGCTGCCCGATGCACTGAAAACGGTGTTGAAATGGTGGGCTGAGG |
| | GGCAGGCATTCATGAGTGGCGCTGCCTCAACCGGGAAGGGCCGATTCCGGATGG |
| | AAAATGCTAAATATGAAACGCTGGATCTGAGCGACGAGAATCAAAGGAATGACTAT |
| | CTTAAGAATTGGGGATGGCGTGACGAGAAGGGGCTCGAGGAACTGAAGAAACGA |
| | CTGAACTCAGGTCTGCCAGAGCCCGGTAATTATAGGGATCCAAAATGGCACGAGA |
| | TTAACGTTTCCATTGAGATGGCAAGCCCTTTTATTAATGGCGACCCAATCCGCGCA |
| | GCCGTGGACAAACGTGGTACAgatGTGGTTACCTTCGTTAAGTATAAAGCTGAAGG |
| | GGAAGAGGCGAAACCCGTATGTGCATACAAGGCCGAATCTTTTAGAGGGGTGATC |
| | AGAAGTGCCGTGGCACGCATTCATATGGAAGATGGCGTCCCTTTGACTGAGTTGA |
| | CTCACAGTGACTGTGAATGTCTCCTGTGCCAAATCTTTGGAAGTGAGTATGAAGCC |
| | GGCAAAATAAGGTTTGAAGATCTCGTATTCGAAAGTGACCCGGAACCTGTGACCTT |
| | CGATCATGTGGCCATCGATAGATTCACTGGCGGTGCAGCTGATAAGAAGAAATTC |
| | GATGATTCCCCTCTGCCCGGTAGCCCTGCAAGACCGCTCATGTTGAAAGGCTCCT |
| | TCTGGATCCGCAGGGACGTTCTCGAGGACGAAGAGTACTGTAAGGCACTCGGTAA |
| | GGCTCTTGCAGATGTGAATAATGGCCTTTATCCCCTCGGTGGAAAGAGCGCCATC |
| | GGCTACGGACAGGTCAAGAGTCTGGGTATAAAGGGAGATGATAAGAGGATTTCTC |
| | GCCTCATGAATCCTGCCTTTGATGAGACAGATGTAGCCGTTCCAGAAAAGCCCAAA |
| | ACTGATGCCGAGGTTCGCATCGAGGCAGAGAAAGTATATTACCCACACTATTTCGT |
| | CGAACCCCATAAGAAGGTGGAACGCGAGGAGAAACCCTGTGGTCATCAAAAGTTC |
| | CACGAGGGGCGACTGACAGGTAAAATTCGGTGTAAGCTCATTACCAAGACACCCC |
| | TCATCGTCCCAGATACTAGTAATGACGATTTCTTCAGACCTGCGGATAAAGAAGCT |
| | CGGAAGGAAAAGGACGAATATCATAAATCATATGCTTTCTTCAGACTTCATAAACAA |
| | ATCATGATTCCCGGGAGCGAATTGAGAGGAATGGTGAGTAGTGTCTACGAAACTG |
| | TGACAAATTCTTGCTTCAGGATATTTGATGAGACTAAACGGTTGTCATGGCGGATG |
| | GATGCTGATCACCAGAATGTGCTGCAAGACTTTCTCCCAGGTCGAGTGACCGCCG |
| | ATGGGAAACATATACAAAAGTTTTCCGAAACTGCAAGGGTGCCTTTCTATGACAAA |
| | ACGCAGAAACATTTTGACATTCTTGATGAGCAAGAAATTGCTGGTGAGAAACCTGT |
| | TCGGATGTGGGTCAAGCGTTTTATTAAACGACTGAGCCTCGTTGATCCTGCTAAAC |
| | ACCCCCAGAAGAAACAAGACAATAAATGGAAAAGACGCAAAGAAGGCATCGCCAC |
| | ATTTATAGAGCAAAAGAATGGCTCTTATTATTTTAACGTGGTCACCAATAATGGCTG |
| | CACTTCTTTCCACTTGTGGCATAAACCTGACAATTTTGACCAGGAGAAACTCGAAG |
| | GCATACAGAATGGTGAGAAACTGGATTGCTGGGTAAGAGATAGTAGATACCAAAA |
| | GGCCTTTCAAGAGATACCCGAGAACGACCCAGACGGATGGGAGTGTAAAGAGGG |
| | CTACCTTCATGTCGTCGGCCCCAGCAAAGTAGAGTTTAGTGACAAGAAAGGGGAT |
| | GTGATCAATAACTTTCAAGGAACACTCCCATCAGTTCCCAACGACTGGAAAACAAT |
| | TAGGACGAACGATTTTAAGAATAGGAAAAGGAAGAATGAACCTGTGTTTTGTTGTG |
| | AGGATGATAAGGGCAATTACTATACCATGGCTAAATATTGCGAAACCTTCTTCTTC |
| | GATCTGAAGGAGAACGAGGAATACGAGATCCCCGAGAAAGCAAGAATCAAATACA |
| | AAGAACTGCTCAGGGTCTATAACAACAATCCTCAAGCAGTGCCGGAGAGCGTATTT |
| | CAGTCTAGAGTTGCCCGGGAAAACGTGGAAAAGCTGAAGTCCGGAGATCTTGTGT |
| | ATTTCAAACATAATGAAAAGTACGTAGAGGACATCGTCCCAGTGCGGATTTCCCGA |
| | ACTGTAGACGATAGGATGATCGGCAAACGTATGAGCGCCGATCTGCGGCCGTGC |
| | CATGGAGATTGGGTGGAAGATGGTGATCTCAGTGCCTTGAATGCATATCCCGAGA |
| | AAAGACTCCTCTTGCGCCACCCCAAAGGACTCTGCCCTGCTTGCCGGCTCTTTGG |
| | AACCGGATCTTACAAGGGCAGAGTCAGGTTTGGATTCGCGTCACTCGAAAACGAT |
| | CCGGAGTGGCTGATCCCAGGCAAGAATCCCGGCGATCCGTTTCACGGCGGGCCG |
| | GTGATGCTCTCATTGTTGGAACGGCCTCGCCCGACTTGGAGTATACCGGGATCCG |
| | ACAATAAGTTTAAAGTGCCTGGCAGAAAGTTTTACGTCCACCACCACGCCTGGAAA |
| | ACCATTAAGGACGGGAACCATCCCACAACAGGCAAAGCTATTGAACAAAGCCCTA |
| | ATAACCGCACTGTAGAAGCTCTCGCCGGCGGGAATTCCTTTAGCTTCGAAATTGC |
| | CTTTGAGAACCTGAAAGAATGGGAGCTGGGTTTGCTCATCCACAGCCTGCAACTC |
| | GAAAAGGGTCTGGCGCATAAACTTGGAATGGCAAAGTCTATGGGATTTGGTTCAG |
| | TTGAAATTGACGTCGAATCAGTGCGCCTGAGAAAAGATTGGAAGCAATGGCGGAA |
| | TGGCAATTCCGAAATTCCCAACTGGTTGGGAAAAGGATTTGCTAAACTGAAGGAAT |
| | GGTTCCGGGACGAGCTCGATTTTATAGAAAATCTTAAGAAACTTCTTTGGTTTCCT |
| | GAGGGCGACCAAGCACCCCGGGTTTGCTACCCCATGCTGCGAAAGAAGGACGAT |
| | CCTAATGGGAATAGCGGTTACGAAGAACTCAAAAGGGGGGAATTCAAGAAAGAAG |
| | ATCGGCAGAAGAAGCTGACCACGCCGTGGACACCGTGGGCATAG |

Mutants were nominated from the solved structure of Desulfonema ishimotonii and generated by site directed mutagenesis with primers, then assembled by Gibson assembly. Cloned variants were transformed into E. coli, grown overnight, then picked into TB media for outgrowth. Following an outgrowth period, a Qiagen 96well-miniprep protocol was used to purify plasmid and correct cloning was confirmed through Tn5 fragmentation and sequencing on an Illumina MiSeq. All constructs were transfected at a 96-well scale on HEK293FT cells in DMEM 10% FBS, along with a guide targeting the g-luciferase transcript (SEQ ID NO:73, sequence 5′-TGCAGCCAGCTITCCGGGCATTGGCTTCCAT-3′) and a reporter plasmid expressing Gaussia-luciferase and Cypridina luciferase. For all knockdown experiments, 10 ng of guide, 10 ng of target plasmid, and 40 ng of DiCas7-11 plasmids were co-transfected using Lipofectamine 3000.

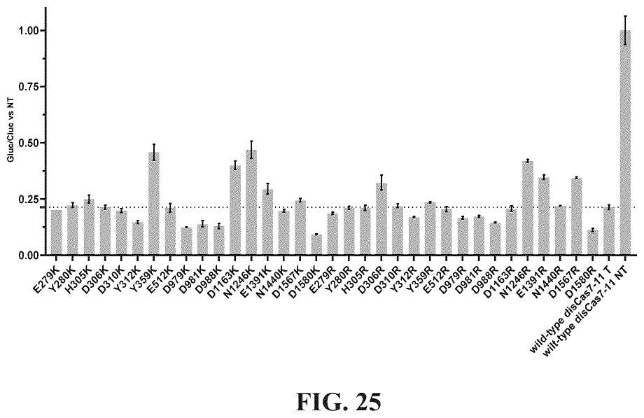

G-luciferase levels were read using a 96-well plate reader 48 h post-transfection and normalized against C-luciferase levels. Results of the readout are shown in FIG. 25.

Endonuclease constructs are shown across the x-axis, the y-axis displays the relative G-luciferase to C-luciferase level for each construct, normalized to a non-targeting guide (SEQ ID NO:74, sequence 5′-GGTAATGCCTGGCTTGTCGACGCATAGTCTG-3′).

### Example 18

### Single Mutant Endogenous MALAT1 Knockdown

Knockdown readout to measure cleavage activity on endogenous MALAT1 transcripts by wildtype and mutant DiCas7-11 endonucleases was performed.

Mutants were nominated from the solved structure of Desulfonema ishimotonii and generated by site directed mutagenesis with primers, then assembled by Gibson assembly. Cloned variants were transformed into E. coli, grown overnight, then picked into TB media for outgrowth. Following an outgrowth period, a Qiagen 96well-miniprep protocol was used to purify plasmid and correct cloning was confirmed through Tn5 fragmentation and sequencing on an Illumina MiSeq. All constructs were transfected at a 96-well scale on HEK293FT cells in DMEM 10% FBS, along with a guide targeting MALAT1 (SEQ ID NO:75, sequence 5′-GGTTATAGCTGACAAGCAATTAACTTAAA-3′). Knockdown transfections combine 10 ng of guide and 40 ng of DiCas7-11 plasmids and are performed using Lipofectamine 3000. RNA was harvested 3 days post-transfection and reverse transcribed using the Thermo RevertAid cDNA prep kid with random hexamer and poly-dT primers. qPCR was performed on all samples using assays targeting endogenous MALAT and GAPDH as a control.

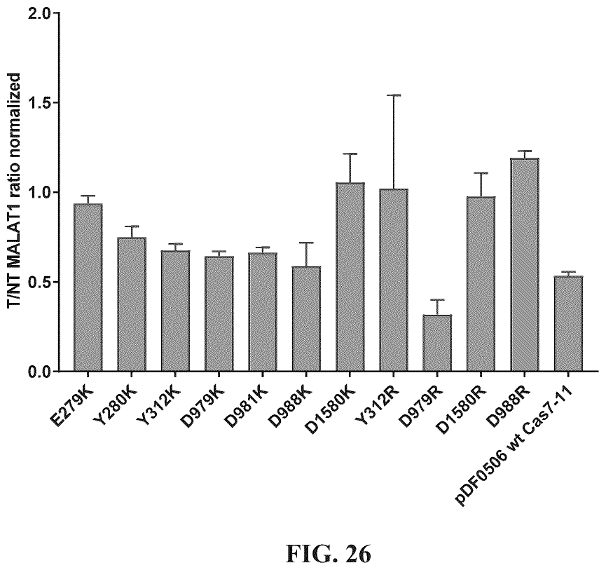

Results of the readout are shown in FIG. 26. Endonuclease constructs are shown across the x-axis, the y-axis displays the relative MALAT expression with each mutant normalized to a non-targeting guide (SEQ ID NO:74, sequence 5′-GGTAATGCCTGGCTTGTCG ACGCATAGTCTG-3′).

### Example 19

### Single Mutant Endogenous PPIB Knockdown

Knockdown readout to measure cleavage activity on transcripts of luminescent Guassia luciferase protein by wildtype and mutant DiCas7-11 endonucleases was performed.

Mutants were nominated from the solved structure of Desulfonema ishimotonii and generated by site directed mutagenesis with primers, then assembled by Gibson assembly. Cloned variants were transformed into E. coli, grown overnight, then picked into TB media for outgrowth. Following an outgrowth period, a Qiagen 96well-miniprep protocol was used to purify plasmid and correct cloning was confirmed through Tn5 fragmentation and sequencing on an Illumina MiSeq. All constructs were transfected at a 96-well scale on HEK293FT cells in DMEM 10% FBS, along with a guide targeting PPIB (SEQ ID NO:76, sequence 5′-cagtgttggtaggagtttgttacaaaagtga-3′). Knockdown transfections combine 10 ng of guide and 40 ng of DiCas7-11 plasmids and are performed using Lipofectamine 3000. RNA was harvested 3 days post-transfection and reverse transcribed using the Thermo RevertAid cDNA prep kid with random hexamer and poly-dT primers. qPCR was performed on all samples using assays targeting endogenous PPIB and GAPDH as a control.

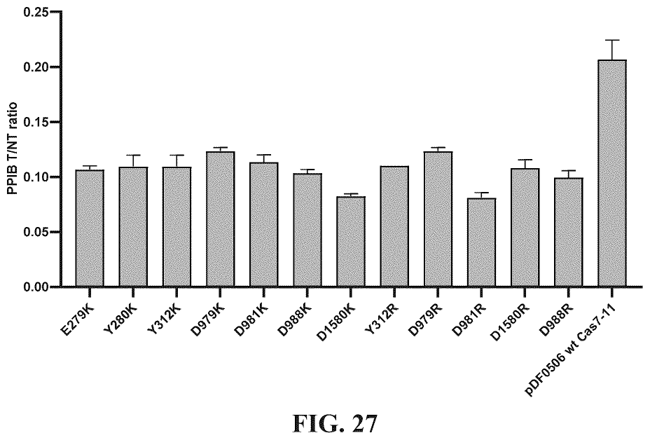

Results of the readout are shown in FIG. 27. Endonuclease constructs are shown across the x-axis, the y-axis displays the relative PPIB expression with each mutant normalized to a non-targeting guide (SEQ ID NO:74, sequence 5′-GGTAATGCCTGGCTTGTCG ACGCATAGTCTG-3′).

### Example 20

### Single and Double Mutant G-Luciferase Knockdown

Knockdown readout to measure cleavage activity on transcripts of luminescent Guassia luciferase protein by wildtype and mutant DiCas7-11 endonucleases was performed.

Mutants were nominated from the solved structure of Desulfonema ishimotonii and generated by site directed mutagenesis with primers, then assembled by Gibson assembly. Cloned variants were transformed into E. coli, grown overnight, then picked into TB media for outgrowth. Following an outgrowth period, a Qiagen 96well-miniprep protocol was used to purify plasmid and correct cloning was confirmed through Tn5 fragmentation and sequencing on an Illumina MiSeq. Double mutants shown here were created by cloning combinations of working mutations using the same method employed for the 1st round hits. All constructs were transfected at a 96-well scale on HEK293FT cells in DMEM 10% FBS, along with a guide targeting the g-luciferase transcript (SEQ ID NO:73, sequence 5′-TGCAGCCAGCTITCCGGGCATTGGCTTCCAT-3′) and a reporter plasmid expressing Gaussia-luciferase and Cypridina luciferase. For all knockdown experiments, 10 ng of guide, 10 ng of target plasmid, and 40 ng of DiCas7-11 plasmids were co-transfected using Lipofectamine 3000. G-luciferase levels were read using a 96-well plate reader 48 h post-transfection and normalized against C-luciferase levels.

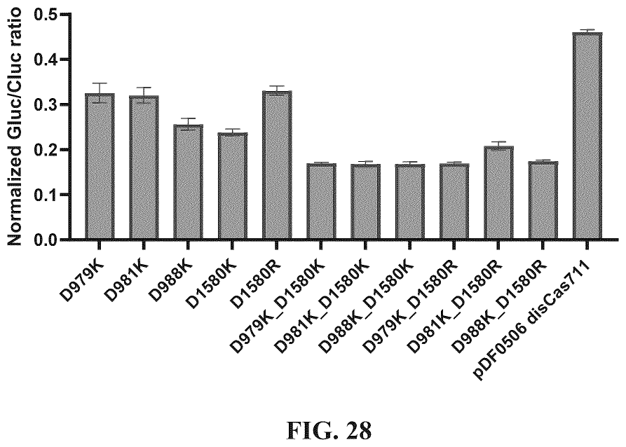

Results of the readout are shown in FIG. 28. Endonuclease constructs are shown across the x-axis, the y-axis displays the relative G-luciferase to C-luciferase level for each construct, normalized to a non-targeting guide (SEQ ID NO:74, sequence 5′-GGTAATGCCTGGCTTGTCG ACGCATAGTCTG-3′).

### Example 21

### Saturation Mutagenesis for D1580 Residue

Knockdown readout to measure cleavage activity on transcripts of luminescent Guassia luciferase protein by wildtype and mutant DiCas7-11 endonucleases was performed.

Mutants generated by site directed mutagenesis for the amino acid residue D1580, which was substituted by each other residue. The small-discas7-11 constructs also shown here are based on the 2021 structure paper from Kato et al. (Kato K, Zhou W, Okazaki S, Isayama Y, Nishizawa T, Gootenberg J S, Abudayyeh O O, Nishimasu H. Structure and engineering of the type III-E CRISPR-Cas7-11 effector complex. Cell. 2022 Jun. 23; 185(13):2324-2337.e16. doi: 10.1016/j.cell.2022.05.003. Epub 2022 May 27. PMID: 35643083) which replaced the Cas7-11 INS domain with a short GS linker sequence. Mutations were also generated on top of the small-cas scaffold. Cloned variants were transformed into E. coli, grown overnight, then picked into TB media for outgrowth. Following an outgrowth period, a Qiagen 96well-miniprep protocol was used to purify plasmid and correct cloning was confirmed through Tn5 fragmentation and sequencing on an Illumina MiSeq. All constructs were transfected at a 96-well scale on HEK293FT cells in DMEM 10% FBS, along with a guide targeting the g-luciferase transcript (SEQ ID NO:73, sequence 5′-TGCAGCCAGCTITCCGGGCATTGGCTTCCAT-3′) and a reporter plasmid expressing Gaussia-luciferase and Cypridina luciferase. For all knockdown experiments, 10 ng of guide 10 ng of target plasmid and 40 ng of DiCas7-11 plasmids were co-transfected using Lipofectamine 3000. G-luciferase levels were read using a 96-well plate reader 48 h post-transfection and normalized against C-luciferase levels.

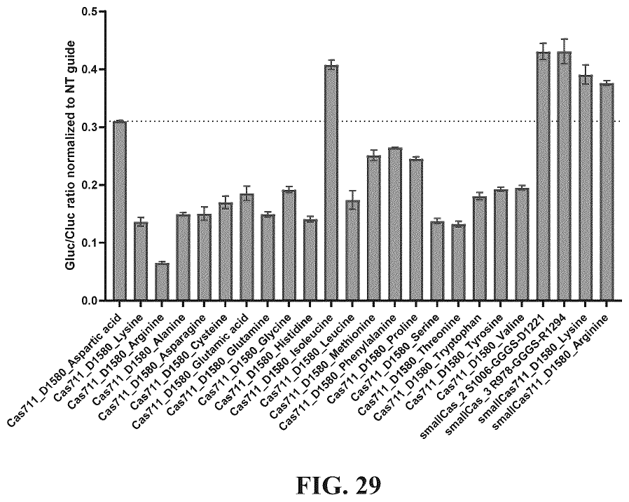

Results of the readout are shown in FIG. 29. Endonuclease constructs are shown across the x-axis, the y-axis displays the relative G-luciferase to C-luciferase level for each construct, normalized to a non-targeting guide (SEQ ID NO:74, sequence 5′-GGTAATGCCTGGCTTGTCG ACGCATAGTCTG-3′).

### Example 22

### Single, Double, Triple, and Quadrupole Mutants G-Luciferase Knockdown

Knockdown readout to measure cleavage activity on transcripts of luminescent Guassia luciferase protein by wildtype and mutant DiCas7-11 endonucleases was performed.

Mutants generated by site directed mutagenesis for the amino acid residue D1580, which was substituted by each other residue. The small-discas7-11 constructs also shown here are based on the 2021 structure paper from Kato et al. (Kato K, Zhou W, Okazaki S, Isayama Y, Nishizawa T, Gootenberg J S, Abudayyeh O O, Nishimasu H. Structure and engineering of the type III-E CRISPR-Cas7-11 effector complex. Cell. 2022 Jun. 23; 185(13):2324-2337.e16. doi: 10.1016/j.cell.2022.05.003. Epub 2022 May 27. PMID: 35643083) which replaced the DiCas7-11 INS domain with a short GS linker sequence. Mutations were also generated on top of the small-cas scaffold. Cloned variants were transformed into E. coli, grown overnight, then picked into TB media for outgrowth. Following an outgrowth period, a Qiagen 96well-miniprep protocol was used to purify plasmid and correct cloning was confirmed through Tn5 fragmentation and sequencing on an Illumina MiSeq. Triple and quadruple mutants (residues as listed) were generated using the same PCR mutagenesis strategy employed for the single and double mutants. All constructs were transfected at a 96-well scale on HEK293FT cells in DMEM 10% FBS, along with a guide targeting the g-luciferase transcript (SEQ ID NO:73, sequence 5′-TGCAGCCAGCTITCCGGGCATTGGCTTCCAT-3′) and a reporter plasmid expressing Gaussia-luciferase and Cypridina luciferase. For all knockdown experiments, 10 ng of guide, 10 ng of target plasmid, and 40 ng of DiCas7-11 plasmids were co-transfected using Lipofectamine 3000. G-luciferase levels were read using a 96-well plate reader 48 h post-transfection and normalized against C-luciferase levels.

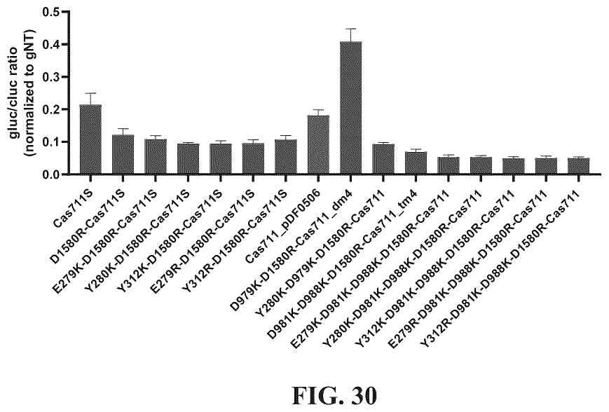

Results of the readout are shown in FIG. 30. Endonuclease constructs are shown across the x-axis, the y-axis displays the relative G-luciferase to C-luciferase level for each construct, normalized to a non-targeting guide (SEQ ID NO:74, sequence 5′-GGTAATGCCTGGCTTGTCG ACGCATAGTCTG-3′).

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

### LIST OF REFERENCES

All publications and references cited herein are expressly incorporated herein by reference in their entirety.

Beljouw, Sam P. B. van, Anna C. Haagsma, Alicia Rodriguez-Molina, Daan F. van den Berg, Jochem N. A. Vink, and Stan J. J. Brouns. 2021. “The gRAMP CRISPR-Cas Effector Is an RNA Endonuclease Complexed with a Caspase-like Peptidase.” Science, August. https://doi.org/10.1126/science.abk2718.

East-Seletsky, Alexandra, Mitchell R. O'Connell, Spencer C. Knight, David Burstein, Jamie H. D. Cate, Robert Tjian, and Jennifer A. Doudna. 2016. “Two Distinct RNase Activities of CRISPR-C2c2 Enable Guide-RNA Processing and RNA Detection.” Nature. https://doi.org/10.1038/nature19802.

Jia, Ning, Charlie Y. Mo, Chongyuan Wang, Edward T. Eng, Luciano A. Marraffini, and Dinshaw J. Patel. 2019. “Type III-A CRISPR-Cas Csm Complexes: Assembly, Periodic RNA Cleavage, DNase Activity Regulation, and Autoimmunity.” Molecular Cell 73 (2): 264-77.e5.

Osawa, Takuo, Hideko Inanaga, Chikara Sato, and Tomoyuki Numata. 2015. “Crystal Structure of the CRISPR-Cas RNA Silencing Cmr Complex Bound to a Target Analog.” Molecular Cell 58 (3): 418-30.

Özcan, Ahsen, Rohan Krajeski, Eleonora Ioannidi, Brennan Lee, Apolonia Gardner, Kira S. Makarova, Eugene V. Koonin, Omar O. Abudayyeh, and Jonathan S. Gootenberg. 2021. “Programmable RNA Targeting with the Single-Protein CRISPR Effector Cas7-11.” Nature, September. https://doi.org/10.1038/s41586-021-03886-5.

Swarts, Daan C., John van der Oost, and Martin Jinek. 2017. “Structural Basis for Guide RNA Processing and Seed-Dependent DNA Targeting by CRISPR-Cas12a.” Molecular Cell 66 (2): 221-33.e4.

Taylor, David W., Yifan Zhu, Raymond H. J. Staals, Jack E. Komfeld, Akeo Shinkai, John van der Oost, Eva Nogales, and Jennifer A. Doudna. 2015. “Structural Biology. Structures of the CRISPR-Cmr Complex Reveal Mode of RNA Target Positioning.” Science 348 (6234): 581-85.

Yang, Wei. 2011. “Nucleases: Diversity of Structure, Function and Mechanism.” Quarterly Reviews of Biophysics 44 (1): 1-93.

You, Lilan, Jun Ma, Jiuyu Wang, Daria Artamonova, Min Wang, Liang Liu, Hua Xiang, Konstantin Severinov, Xinzheng Zhang, and Yanli Wang. 2019. “Structure Studies of the CRISPR-Csm Complex Reveal Mechanism of Co-Transcriptional Interference.” Cell 176 (1-2): 239-53.e16.

Kato K, Zhou W, Okazaki S, Isayama Y, Nishizawa T, Gootenberg J S, Abudayyeh O O, Nishimasu H. Structure and engineering of the type III-E CRISPR-Cas7-11 effector complex. Cell. 2022 Jun. 23; 185(13):2324-2337.e16. doi: 10.1016/j.cell.2022.05.003. Epub 2022 May 27. PMID: 35643083

1. A genome editing system capable of cleaving an RNA target, the system comprising:
a polypeptide comprising an amino acid sequence at least 85% identical to the amino acid sequence of any one of SEQ ID NOs: 1-4 wherein the amino acid sequence of the polypeptide comprises at least one amino acid modification or mutation relative to the amino acid sequence of SEQ ID NO: 1-4,
a deaminase, and
a guide RNA that hybridizes to the RNA target and the polypeptide.

2-6. (canceled)

7. The genome editing system of claim 1, wherein the at least one amino acid modification or mutation comprises:
removing an amino acid;
adding an amino acid;
replacing an amino acid with no charge with an amino acid with a positive charge; or
replacing an amino acid with a negative charge with an amino acid with a positive charge.

8. The genome editing system of claim 7, wherein:
the amino acid without charge is selected from the group consisting of serine, threonine, asparagine, glutamine, cysteine, glycine, proline, alanine, valine, isoleucine, leucine, methionine, phenylalanine, tyrosine, and tryptophan;
the amino acid with a negative charge is selected from the group consisting of aspartic acid and glutamic acid: or
the amino acid with a positive charge is selected from the group consisting of arginine, histidine, and lysine.

9-10. (canceled)

11. The genome editing system of claim 1, wherein the amino acid sequence of the polypeptide comprises 1, 2, 3, or 4 amino acid modifications or mutations.

12. The genome editing system of claim 1, wherein the amino acid sequence of the polypeptide comprises an alanine at a position corresponding to position 43 of SEQ ID NO: 1; an alanine at a position corresponding to position 55 of SEQ ID NO: 55; and/or an alanine at a position corresponding to position 152 of SEQ ID NO: 1.

13. The genome editing system of claim 1, wherein the polypeptide comprises a deletion of one or more amino acid residues at positions 979 through 1293 of SEQ ID NO: 1; at positions 1007 through 1220 of SEQ ID NO: 1; and/or at positions 1146 through 1211 of SEQ ID NO: 1.

14. A composition that cleaves an RNA target comprising:
a polypeptide comprising an amino acid sequence at least 85% identical to the amino acid sequence of any one of SEO ID NOs: 1-4 wherein the amino acid sequence of the polypeptide comprises at least one amino acid modification or mutation relative to the amino acid sequence of SEO ID NO: 1-4,
a deaminase, and
a guide RNA that hybridizes to the RNA target and the polypeptide.

15. The composition of claim 14, wherein:
the guide RNA comprises a mismatch distance that is about 20-65% of the length of the guide;
the guide RNA has a sequence with a length of about 20 to about 53 nucleotides (nt), optionally about 25 to about 53 nt, optionally about 29 to about 53 nt, or optionally about 40 to about 50 nt;
the guide RNA is a pre-crRNA;
the guide RNA is a mature crRNA;
the RNA target is a single-strand RNA (ssRNA);
the RNA target is in a cell, optionally wherein the cell is a prokaryotic cell or eukaryotic cell, optionally wherein the eukaryotic cell is a mammalian cell, optionally wherein the mammalian cell is a human cell; or
the guide RNA comprises a mismatch that is about 20 to about 30 nucleotides from a non-pairing C of the guide RNA.

16-25. (canceled)

26. One or more nucleic acid molecules encoding the polypeptide, the deaminase, and the guide RNA of claim 14.

27-28. (canceled)

29. A vector comprising the one of more nucleic acid molecules of claim 26, wherein the vector is a viral vector,
optionally wherein the viral vector is a lenti-associated viral vector, baculo-associated viral vector, or adeno-associated viral vector, and
optionally wherein the viral vector is derived from a virus selected from the group consisting of Myoviridae, Siphoviridae, Podoviridae, Corticoviridae, Lipothrixviridae, Poxviridae, Iridoviridae, Adenoviridae, Polyomaviridae, Papillomaviridae, Mimiviridae, Pandoravirusa, Salterprovirusa, Inoviridae, Microviridae, Parvoviridae, Circoviridae, Hepadnaviridae, Caulimoviridae, Retroviridae, Cystoviridae, Reoviridae, Birnaviridae, Totiviridae, Partitiviridae, Filoviridae, Orthomyxoviridae, Deltavirusa, Leviviridae, Picornaviridae, Marnaviridae, Secoviridae, Potyviridae, Caliciviridae, Hepeviridae, Astroviridae, Nodaviridae, Tetraviridae, Luteoviridae, Tombusviridae, Coronaviridae, Arteriviridae, Flaviviridae, Togaviridae, Virgaviridae, Bromoviridae, Tymoviridae, Alphaflexiviridae, Sobemovirusa, Idaeovirusa, and Herpesviridae.

30. A cell comprising the composition of claim 14, optionally wherein:
the cell is a prokaryotic cell, or
the cell is a eukaryotic cell, optionally wherein the eukaryotic cell is a mammalian cell, optionally wherein the mammalian cell is a human cell.

31-34. (canceled)

35. A method of cleaving an RNA target in a cell, stabilizing an RNA target in a cell, or affecting translation of an RNA target in a cell, the method comprising providing to the cell the composition of claim 14, optionally wherein the RNA target is an ssRNA.

36-38. (canceled)

39. A method of treating a genetically inherited disease in a subject in need thereof comprising administering to the subject an effective amount of the composition of claim 14, wherein the genetically inherited disease involves a guanosine to adenosine change in a genome of the subject or is a pre-termination disease.

40. The method of claim 39, wherein the genetically inherited disease is selected from the group consisting of Meier-Gorlin syndrome; Seckel syndrome 4; Joubert syndrome 5; Leber congenital amaurosis 10; Charcot-Marie-Tooth disease, type 2; leukoencephalopathy; Usher syndrome, type 2C; spinocerebellar ataxia 28; glycogen storage disease type III; primary hyperoxaluria, type I; long QT syndrome 2; Sjögren-Larsson syndrome; hereditary fructosuria; neuroblastoma; amyotrophic lateral sclerosis type 9; Kallmann syndrome 1; limb-girdle muscular dystrophy, type 2L; familial adenomatous polyposis 1; familial type 3 hyperlipoproteinemia; Alzheimer's disease, type 1; metachromatic leukodystrophy; cancer; Uveitis; SCA1; SCA2; FUS-Amyotrophic Lateral Sclerosis (ALS); MAPT-Frontotemporal Dementia (FTD); Myotonic Dystrophy Type 1 (DM1); Diabetic Retinopathy (DR/DME); Oculopharyngeal Muscular Dystrophy (OPMD); SCA8; C90RF72-Amyotrophic Lateral Sclerosis (ALS); SOD1-Amyotrophic Lateral Sclerosis (ALS); Spinal Cord Injury (targets: mTOR, PTEN, KLF6/7, SOX11, KCC2, and growth factors); SCA6; SCA3 (Machado-Joseph Disease); Multiple system Atrophy (MSA); Treatment-resistant Hypertension; Myotonic Dystrophy Type 2 (DM2); Fragile X-associated Tremor Ataxia Syndrome (FXTAS); West Syndrome with ARX Mutation; Age-related Macular Degeneration (AMD)/Geographic Atrophy (GA); C90RF72-Frontotemporal Dementia (FTD); Facioscapulohumeral Muscular Dystrophy (FSHD); Fragile X Syndrome (FXS); Huntington's Disease; Glaucoma; Acromegaly; Achromatopsia (total color blindness); Ullrich congenital muscular dystrophy; Hereditary myopathy with lactic acidosis; X-linked spondyloepiphyseal dysplasia tarda; Neuropathic pain (Target: CPEB); Persistent Inflammation and injury pain (Target: PABP); Neuropathic pain (Target: miR-30c-5p); Neuropathic pain (Target: miR-195); Friedreich's Ataxia; Uncontrolled gout; Inflammatory pain (Target: Nav1.7 and Nav1.8); Choroideremia; Focal epilepsy; Alpha-1 Antitrypsin deficiency (AATD); Androgen Insensitivity Syndrome; Opioid-induced hyperalgesia (Target: Raf-1); Neurofibromatosis type 1; Stargardt's Disease; Dravet Syndrome; Retinitis Pigmentosa; and Parkinson's Disease.

41. (canceled)

42. A method of altering splicing of a pre-mRNA in a cell, increasing RNA stability in a cell, or modulating translation in a cell, the method comprising administering to the cell an effective amount of the composition of claim 14.

43. A method of changing microRNA targets in a subject in need thereof comprising administering to the subject an effective amount of the composition of claim 14.

44-45. (canceled)

46. A method of detecting a bacterium or derivative thereof in a sample, the method comprising:
adding to the sample an effective amount of the composition of claim 14; and
detecting a reporter specific to the bacterium or derivative thereof.

47. A method of detecting a virus or derivative thereof in a sample, the method comprising:
adding to the sample an effective amount of the compositions of claim 14; and
detecting a reporter specific to the virus or derivative thereof.

48. The genome editing system of claim 1, wherein the deaminase is selected from the group consisting of an adenosine deaminase or a catalytic domain thereof, and a cytidine deaminase or a catalytic domain thereof.

49. A genome editing system capable of cleaving an RNA target, the system comprising:
a polypeptide comprising an amino acid sequence at least 85% identical to the amino acid sequence of any one of SEQ ID NOs: 1-4, wherein the polypeptide comprises a deaminase that is fused or linked to the polypeptide, and wherein the amino acid sequence of the polypeptide comprises at least one amino acid modification or mutation relative to the amino acid sequence of SEQ ID NO: 1-4, and
a guide RNA that hybridizes to the RNA target and the polypeptide.


SEQUENCE LISTING (152 sequences)

SEQ ID NO: 1 - protein, 1601 aa, synthetic construct
  REGION 1..1601: note = huDiCas7-11
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 2 - protein, 1290 aa, synthetic construct
  REGION 1..1290: note = huDiCas7-11-R978-GGGS-R1294
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRGGGSRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 3 - protein, 1391 aa, synthetic construct
  REGION 1..1391: note = huDiCas7-11-S1006-GGGS-D1221
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSGGGSDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 4 - protein, 1529 aa, synthetic construct
  REGION 1..1529: note = huDiCas7-11-R1045-GGGS-R1122
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRGGGSRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 5 - RNA, 27 nt, synthetic construct
  misc_feature 1..27: note = tgRNA_for structural analysis
  source 1..27: mol_type = other RNA
ggattacccatgtcgaagacaacaaag

SEQ ID NO: 6 - RNA, 62 nt, synthetic construct
  misc_feature 1..62: note = 60-nt processing target
  source 1..62: mol_type = other RNA
cyggggttggaaagccggttttctttgatgtcacggaacctttgttgtcttcgacatgggta

SEQ ID NO: 7 - RNA, 62 nt, synthetic construct
  misc_feature 1..62: note = 60-nt MS2 ssRNA target
  source 1..62: mol_type = other RNA
cyccggccattcaaacatgaggattacccatgtcgaagacaacaaagaagttcaactcttta

SEQ ID NO: 8 - RNA, 41 nt, synthetic construct
  misc_feature 1..41: note = crRNA_for structural analysis
  source 1..41: mol_type = other RNA
  ncRNA 1..41: ncRNA_class = guide_RNA
ggtgatgtcacggaacctttgttgtcttcgacatgggtaat

SEQ ID NO: 9 - RNA, 48 nt, synthetic construct
  misc_feature 1..48: note = Mature crRNA for MS2 ssRNA target
  source 1..48: mol_type = other RNA
  ncRNA 1..48: ncRNA_class = guide_RNA
ggttgatgtcacggaacctttgttgtcttcgacatgggtaatcctcat

SEQ ID NO: 10 - RNA, 68 nt, synthetic construct
  misc_feature 1..68: note = Pre-mature crRNA for MS2 ssRNA target, WT
  source 1..68: mol_type = other RNA
  ncRNA 1..68: ncRNA_class = guide_RNA
ggggttggaaagccggttttctttgatgtcacggaacctttgttgtcttcgacatgggtaatcctcat

SEQ ID NO: 11 - RNA, 68 nt, synthetic construct
  misc_feature 1..68: note = Pre-mature crRNA for MS2 ssRNA target, mismatch 1
  source 1..68: mol_type = other RNA
  ncRNA 1..68: ncRNA_class = guide_RNA
ggggttggaaagccggttttctttgatgtcacggaacgtttgttgtcttcgacatgggtaatcctcat

SEQ ID NO: 12 - RNA, 68 nt, synthetic construct
  misc_feature 1..68: note = Pre-mature crRNA for MS2 ssRNA target, mismatch 2
  source 1..68: mol_type = other RNA
  ncRNA 1..68: ncRNA_class = guide_RNA
ggggttggaaagccggttttctttgatgtcacggaaccattgttgtcttcgacatgggtaatcctcat

SEQ ID NO: 13 - RNA, 68 nt, synthetic construct
  misc_feature 1..68: note = Pre-mature crRNA for MS2 ssRNA target, mismatch 3
  source 1..68: mol_type = other RNA
  ncRNA 1..68: ncRNA_class = guide_RNA
ggggttggaaagccggttttctttgatgtcacggaacctatgttgtcttcgacatgggtaatcctcat

SEQ ID NO: 14 - RNA, 68 nt, synthetic construct
  misc_feature 1..68: note = Pre-mature crRNA for MS2 ssRNA target, mismatch 4
  source 1..68: mol_type = other RNA
  ncRNA 1..68: ncRNA_class = guide_RNA
ggggttggaaagccggttttctttgatgtcacggaaccttagttgtcttcgacatgggtaatcctcat

SEQ ID NO: 15 - RNA, 68 nt, synthetic construct
  misc_feature 1..68: note = Pre-mature crRNA for MS2 ssRNA target, mismatch 5
  source 1..68: mol_type = other RNA
  ncRNA 1..68: ncRNA_class = guide_RNA
ggggttggaaagccggttttctttgatgtcacggaacctttcttgtcttcgacatgggtaatcctcat

SEQ ID NO: 16 - RNA, 68 nt, synthetic construct
  misc_feature 1..68: note = Pre-mature crRNA for MS2 ssRNA target, mismatch 6
  source 1..68: mol_type = other RNA
  ncRNA 1..68: ncRNA_class = guide_RNA
ggggttggaaagccggttttctttgatgtcacggaacctttgatgtcttcgacatgggtaatcctcat

SEQ ID NO: 17 - RNA, 68 nt, synthetic construct
  misc_feature 1..68: note = Pre-mature crRNA for MS2 ssRNA target, mismatch 7
  source 1..68: mol_type = other RNA
  ncRNA 1..68: ncRNA_class = guide_RNA
ggggttggaaagccggttttctttgatgtcacggaacctttgtagtcttcgacatgggtaatcctcat

SEQ ID NO: 18 - RNA, 68 nt, synthetic construct
  misc_feature 1..68: note = Pre-mature crRNA for MS2 ssRNA target, mismatch 8
  source 1..68: mol_type = other RNA
  ncRNA 1..68: ncRNA_class = guide_RNA
ggggttggaaagccggttttctttgatgtcacggaacctttgttctcttcgacatgggtaatcctcat

SEQ ID NO: 19 - RNA, 68 nt, synthetic construct
  misc_feature 1..68: note = Pre-mature crRNA for MS2 ssRNA target, mismatch 9
  source 1..68: mol_type = other RNA
  ncRNA 1..68: ncRNA_class = guide_RNA
ggggttggaaagccggttttctttgatgtcacggaacctttgttgacttcgacatgggtaatcctcat

SEQ ID NO: 20 - RNA, 68 nt, synthetic construct
  misc_feature 1..68: note = Pre-mature crRNA for MS2 ssRNA target, mismatch 10
  source 1..68: mol_type = other RNA
  ncRNA 1..68: ncRNA_class = guide_RNA
ggggttggaaagccggttttctttgatgtcacggaacctttgttgtgttcgacatgggtaatcctcat

SEQ ID NO: 21 - RNA, 68 nt, synthetic construct
  misc_feature 1..68: note = Pre-mature crRNA for MS2 ssRNA target, mismatch 11
  source 1..68: mol_type = other RNA
  ncRNA 1..68: ncRNA_class = guide_RNA
ggggttggaaagccggttttctttgatgtcacggaacctttgttgtcatcgacatgggtaatcctcat

SEQ ID NO: 22 - RNA, 68 nt, synthetic construct
  misc_feature 1..68: note = Pre-mature crRNA for MS2 ssRNA target, mismatch 12
  source 1..68: mol_type = other RNA
  ncRNA 1..68: ncRNA_class = guide_RNA
ggggttggaaagccggttttctttgatgtcacggaacctttgttgtctacgacatgggtaatcctcat

SEQ ID NO: 23 - RNA, 68 nt, synthetic construct
  misc_feature 1..68: note = Pre-mature crRNA for MS2 ssRNA target, mismatch 13
  source 1..68: mol_type = other RNA
  ncRNA 1..68: ncRNA_class = guide_RNA
ggggttggaaagccggttttctttgatgtcacggaacctttgttgtcttggacatgggtaatcctcat

SEQ ID NO: 24 - RNA, 68 nt, synthetic construct
  misc_feature 1..68: note = Pre-mature crRNA for MS2 ssRNA target, mismatch 14
  source 1..68: mol_type = other RNA
  ncRNA 1..68: ncRNA_class = guide_RNA
ggggttggaaagccggttttctttgatgtcacggaacctttgttgtcttccacatgggtaatcctcat

SEQ ID NO: 25 - RNA, 68 nt, synthetic construct
  misc_feature 1..68: note = Pre-mature crRNA for MS2 ssRNA target, mismatch 15
  source 1..68: mol_type = other RNA
  ncRNA 1..68: ncRNA_class = guide_RNA
ggggttggaaagccggttttctttgatgtcacggaacctttgttgtcttcgtcatgggtaatcctcat

SEQ ID NO: 26 - RNA, 68 nt, synthetic construct
  misc_feature 1..68: note = Pre-mature crRNA for MS2 ssRNA target, mismatch 16
  source 1..68: mol_type = other RNA
  ncRNA 1..68: ncRNA_class = guide_RNA
ggggttggaaagccggttttctttgatgtcacggaacctttgttgtcttcgagatgggtaatcctcat

SEQ ID NO: 27 - RNA, 68 nt, synthetic construct
  misc_feature 1..68: note = Pre-mature crRNA for MS2 ssRNA target, mismatch 17
  source 1..68: mol_type = other RNA
  ncRNA 1..68: ncRNA_class = guide_RNA
ggggttggaaagccggttttctttgatgtcacggaacctttgttgtcttcgacttgggtaatcctcat

SEQ ID NO: 28 - RNA, 68 nt, synthetic construct
  misc_feature 1..68: note = Pre-mature crRNA for MS2 ssRNA target, mismatch 18
  source 1..68: mol_type = other RNA
  ncRNA 1..68: ncRNA_class = guide_RNA
ggggttggaaagccggttttctttgatgtcacggaacctttgttgtcttcgacaagggtaatcctcat

SEQ ID NO: 29 - RNA, 68 nt, synthetic construct
  misc_feature 1..68: note = Pre-mature crRNA for MS2 ssRNA target, mismatch 19
  source 1..68: mol_type = other RNA
  ncRNA 1..68: ncRNA_class = guide_RNA
ggggttggaaagccggttttctttgatgtcacggaacctttgttgtcttcgacatcggtaatcctcat

SEQ ID NO: 30 - RNA, 68 nt, synthetic construct
  misc_feature 1..68: note = Pre-mature crRNA for MS2 ssRNA target, mismatch 20
  source 1..68: mol_type = other RNA
  ncRNA 1..68: ncRNA_class = guide_RNA
ggggttggaaagccggttttctttgatgtcacggaacctttgttgtcttcgacatgcgtaatcctcat

SEQ ID NO: 31 - RNA, 68 nt, synthetic construct
  misc_feature 1..68: note = Pre-mature crRNA for MS2 ssRNA target, mismatch 21
  source 1..68: mol_type = other RNA
  ncRNA 1..68: ncRNA_class = guide_RNA
ggggttggaaagccggttttctttgatgtcacggaacctttgttgtcttcgacatggctaatcctcat

SEQ ID NO: 32 - RNA, 68 nt, synthetic construct
  misc_feature 1..68: note = Pre-mature crRNA for MS2 ssRNA target, mismatch 22
  source 1..68: mol_type = other RNA
  ncRNA 1..68: ncRNA_class = guide_RNA
ggggttggaaagccggttttctttgatgtcacggaacctttgttgtcttcgacatgggaaatcctcat

SEQ ID NO: 33 - RNA, 68 nt, synthetic construct
  misc_feature 1..68: note = Pre-mature crRNA for MS2 ssRNA target, mismatch 23
  source 1..68: mol_type = other RNA
  ncRNA 1..68: ncRNA_class = guide_RNA
ggggttggaaagccggttttctttgatgtcacggaacctttgttgtcttcgacatgggttatcctcat

SEQ ID NO: 34 - RNA, 68 nt, synthetic construct
  misc_feature 1..68: note = Pre-mature crRNA for MS2 ssRNA target, mismatch 3/9
  source 1..68: mol_type = other RNA
  ncRNA 1..68: ncRNA_class = guide_RNA
ggggttggaaagccggttttctttgatgtcacggaacctatgttgacttcgacatgggtaatcctcat

SEQ ID NO: 35 - RNA, 47 nt, synthetic construct
  misc_feature 1..47: note = Gluc targeting guide, WT
  source 1..47: mol_type = other RNA
  ncRNA 1..47: ncRNA_class = guide_RNA
gttgatgtcacggaactgcagccagctttccgggcattggcttccat

SEQ ID NO: 36 - RNA, 47 nt, synthetic construct
  misc_feature 1..47: note = Non-targeting guide
  source 1..47: mol_type = other RNA
  ncRNA 1..47: ncRNA_class = guide_RNA
gttgatgtcacggaacggtaatgcctggcttgtcgacgcatagtctg

SEQ ID NO: 37 - RNA, 47 nt, synthetic construct
  misc_feature 1..47: note = PPIB targeting guide
  source 1..47: mol_type = other RNA
  ncRNA 1..47: ncRNA_class = guide_RNA
gttgatgtcacggaaccagtgttggtaggagtttgttacaaaagtga

SEQ ID NO: 38 - RNA, 47 nt, synthetic construct
  misc_feature 1..47: note = MALAT1 targeting guide
  source 1..47: mol_type = other RNA
  ncRNA 1..47: ncRNA_class = guide_RNA
gttgatgtcacggaacggttatagcttgacaagcaattaactttaaa

SEQ ID NO: 39 - DNA, 34 bp, synthetic construct
  source 1..34: mol_type = other DNA
gataataagaaaaccaagtatacccgcctgcttg

SEQ ID NO: 40 - DNA, 23 bp, synthetic construct
  source 1..23: mol_type = other DNA
gaaaaccgaaaagacccgcctgc

SEQ ID NO: 41 - DNA, 25 bp, synthetic construct
  source 1..25: mol_type = other DNA
tgccaaaggataaggacgggaagga

SEQ ID NO: 42 - DNA, 29 bp, synthetic construct
  source 1..29: mol_type = other DNA
gccaaaggatcataaggggaaggatgacc

SEQ ID NO: 43 - DNA, 28 bp, synthetic construct
  source 1..28: mol_type = other DNA
gacgggaaggataagcattacctctggg

SEQ ID NO: 44 - DNA, 30 bp, synthetic construct
  source 1..30: mol_type = other DNA
gaaggatgaccataagctctgggatatcgg

SEQ ID NO: 45 - DNA, 27 bp, synthetic construct
  source 1..27: mol_type = other DNA
ggtgaagcgctgaagctcaagagtaaa

SEQ ID NO: 46 - DNA, 19 bp, synthetic construct
  source 1..19: mol_type = other DNA
cggttgcgaagggtgccct

SEQ ID NO: 47 - DNA, 22 bp, synthetic construct
  source 1..22: mol_type = other DNA
tggcggatgaaggctgatcacc

SEQ ID NO: 48 - DNA, 27 bp, synthetic construct
  source 1..27: mol_type = other DNA
cggatggatgctaagcaccagaatgtg

SEQ ID NO: 49 - DNA, 26 bp, synthetic construct
  source 1..26: mol_type = other DNA
atgtgctgcaaaagtttctcccaggt

SEQ ID NO: 50 - DNA, 36 bp, synthetic construct
  source 1..36: mol_type = other DNA
tagtgacaagaaagggaaggtgatcaataactttca

SEQ ID NO: 51 - DNA, 31 bp, synthetic construct
  source 1..31: mol_type = other DNA
tcagggtctataacaagaatcctcaagcagt

SEQ ID NO: 52 - DNA, 22 bp, synthetic construct
  source 1..22: mol_type = other DNA
tcattgttgaagcggcctcgcc

SEQ ID NO: 53 - DNA, 24 bp, synthetic construct
  source 1..24: mol_type = other DNA
acaaagccctaagaaccgcactgt

SEQ ID NO: 54 - DNA, 29 bp, synthetic construct
  source 1..29: mol_type = other DNA
gctgcgaaagaagaaggatcctaatggga

SEQ ID NO: 55 - DNA, 31 bp, synthetic construct
  source 1..31: mol_type = other DNA
acgaagaactcaaaaagggggaattcaagaa

SEQ ID NO: 56 - DNA, 28 bp, synthetic construct
  source 1..28: mol_type = other DNA
aataagaaaaccaggtatacccgcctgc

SEQ ID NO: 57 - DNA, 22 bp, synthetic construct
  source 1..22: mol_type = other DNA
aaaaccgaaaggacccgcctgc

SEQ ID NO: 58 - DNA, 23 bp, synthetic construct
  source 1..23: mol_type = other DNA
gccaaaggatagggacgggaagg

SEQ ID NO: 59 - DNA, 28 bp, synthetic construct
  source 1..28: mol_type = other DNA
ccaaaggatcatagggggaaggatgacc

SEQ ID NO: 60 - DNA, 26 bp, synthetic construct
  source 1..26: mol_type = other DNA
acgggaaggataggcattacctctgg

SEQ ID NO: 61 - DNA, 28 bp, synthetic construct
  source 1..28: mol_type = other DNA
aaggatgaccataggctctgggatatcg

SEQ ID NO: 62 - DNA, 25 bp, synthetic construct
  source 1..25: mol_type = other DNA
gtgaagcgctgaggctcaagagtaa

SEQ ID NO: 63 - DNA, 18 bp, synthetic construct
  source 1..18: mol_type = other DNA
ggttgcgaggggtgccct

SEQ ID NO: 64 - DNA, 21 bp, synthetic construct
  source 1..21: mol_type = other DNA
tggcggatgagggctgatcac

SEQ ID NO: 65 - DNA, 26 bp, synthetic construct
  source 1..26: mol_type = other DNA
ggatggatgctaggcaccagaatgtg

SEQ ID NO: 66 - DNA, 24 bp, synthetic construct
  source 1..24: mol_type = other DNA
tgtgctgcaaaggtttctcccagg

SEQ ID NO: 67 - DNA, 33 bp, synthetic construct
  source 1..33: mol_type = other DNA
agtgacaagaaagggagggtgatcaataacttt

SEQ ID NO: 68 - DNA, 30 bp, synthetic construct
  source 1..30: mol_type = other DNA
cagggtctataacaggaatcctcaagcagt

SEQ ID NO: 69 - DNA, 20 bp, synthetic construct
  source 1..20: mol_type = other DNA
cattgttgaggcggcctcgc

SEQ ID NO: 70 - DNA, 23 bp, synthetic construct
  source 1..23: mol_type = other DNA
acaaagccctaggaaccgcactg

SEQ ID NO: 71 - DNA, 28 bp, synthetic construct
  source 1..28: mol_type = other DNA
ctgcgaaagaagagggatcctaatggga

SEQ ID NO: 72 - DNA, 29 bp, synthetic construct
  source 1..29: mol_type = other DNA
cgaagaactcaaaagaggggaattcaaga

SEQ ID NO: 73 - RNA, 31 nt, synthetic construct
  source 1..31: mol_type = other RNA
  ncRNA 1..31: ncRNA_class = guide_RNA
tgcagccagctttccgggcattggcttccat

SEQ ID NO: 74 - RNA, 31 nt, synthetic construct
  source 1..31: mol_type = other RNA
  ncRNA 1..31: ncRNA_class = guide_RNA
ggtaatgcctggcttgtcgacgcatagtctg

SEQ ID NO: 75 - RNA, 31 nt, synthetic construct
  source 1..31: mol_type = other RNA
  ncRNA 1..31: ncRNA_class = guide_RNA
ggttatagcttgacaagcaattaactttaaa

SEQ ID NO: 76 - RNA, 31 nt, synthetic construct
  source 1..31: mol_type = other RNA
  ncRNA 1..31: ncRNA_class = guide_RNA
cagtgttggtaggagtttgttacaaaagtga

SEQ ID NO: 77 - protein, 4 aa, synthetic construct
GGGS

SEQ ID NO: 78 - protein, 5 aa, synthetic construct
GGGGS

SEQ ID NO: 79 - protein, 4 aa, synthetic construct
TGYV

SEQ ID NO: 80 - protein, 4 aa, synthetic construct
NTTN

SEQ ID NO: 81 - protein, 4 aa, synthetic construct
TRTN

SEQ ID NO: 82 - protein, 4 aa, synthetic construct
TYTV

SEQ ID NO: 83 - protein, 4 aa, synthetic construct
TYCT

SEQ ID NO: 84 - protein, 4 aa, synthetic construct
TYCN

SEQ ID NO: 85 - protein, 4 aa, synthetic construct
TACT

SEQ ID NO: 86 - protein, 4 aa, synthetic construct
TYCC

SEQ ID NO: 87 - protein, 4 aa, synthetic construct
TRTC

SEQ ID NO: 88 - protein, 4 aa, synthetic construct
TATV

SEQ ID NO: 89 - protein, 4 aa, synthetic construct
NTTV

SEQ ID NO: 90 - protein, 4 aa, synthetic construct
TSTG

SEQ ID NO: 91 - protein, 4 aa, synthetic construct
TVTS

SEQ ID NO: 92 - protein, 4 aa, synthetic construct
TYYS

SEQ ID NO: 93 - protein, 4 aa, synthetic construct
TCYS

SEQ ID NO: 94 - protein, 4 aa, synthetic construct
TBYS

SEQ ID NO: 95 - protein, 4 aa, synthetic construct
TNYS

SEQ ID NO: 96 - protein, 4 aa, synthetic construct
TNTN

SEQ ID NO: 97 - protein, 4 aa, synthetic construct
TTCC

SEQ ID NO: 98 - protein, 4 aa, synthetic construct
TCCC

SEQ ID NO: 99 - protein, 4 aa, synthetic construct
TATC

SEQ ID NO: 100 - protein, 4 aa, synthetic construct
TGTG

SEQ ID NO: 101 - protein, 4 aa, synthetic construct
TCTG

SEQ ID NO: 102 - protein, 4 aa, synthetic construct
TYCV

SEQ ID NO: 103 - protein, 4 aa, synthetic construct
TCTC

SEQ ID NO: 104 - protein, 7 aa, synthetic construct
GSGGGGS

SEQ ID NO: 105 - protein, 15 aa, synthetic construct
GGGGSGGGGSGGGGS

SEQ ID NO: 106 - protein, 5 aa, synthetic construct
EAAAK

SEQ ID NO: 107 - protein, 15 aa, synthetic construct
EAAAKEAAAKEAAAK

SEQ ID NO: 108 - protein, 18 aa, synthetic construct
GGSGGSGGSGGSGGSGGS

SEQ ID NO: 109 - protein, 16 aa, synthetic construct
SGSETPGTSESATPES

SEQ ID NO: 110 - protein, 1601 aa, synthetic construct; identical to SEQ ID NO: 1

SEQ ID NO: 111 - protein, 1724 aa, synthetic construct
MKSNDMNITVELTFFEPYRLVEWFDWDARKKSHSAMRGQAFAQWTWKGKGRTAGKSFITGTLVRSAVIKAVEELLSLNNGKWEGVPCCNGSFQTDESKGKKPSFLRKRHTLQWQANNKNICDKEEACPFCILLGRFDNAGKVHERNKDYDIHFSNFDLDHKQEKNDLRLVDIASGRILNRVDFDTGKAKDYFRTWEADYETYGTYTGRITLRNEHAKKLLLASLGFVDKLCGALCRIEVIKKSESPLPSDTKEQSYTKDDTVEVLSEDHNDELRKQAEVIVEAFKQNDKLEKIRILADAIRTLRLHGEGVIEKDELPDGKEERDKGHHLWDIKVQGTALRTKLKELWQSNKDIGWRKFTEMLGSNLYLIYKKETGGVSTRFRILGDTEYYSKAHDSEGSDLFIPVTTPPEGIETKEWIIVGRLKAATPFYFGVQQPSDSIPGKEKKSEDSLVINEHTSFNILLDKENRYRIPRSALRGALRRDLRTAFGSGCNVSLGGQILCNCKVCIEMRRITLKDSVSDFSEPPEIRYRIAKNPGTATVEDGSLFDIEVGPEGLTFPFVLRYRGHKFPEQLSSVIRYWEENDGKNGMAWLGGLDSTGKGRFALKDIKIFEWDLNQKINEYIKERGMRGKEKELLEMGESSLPDGLIPYKFFEERECLFPYKENLKPQWSEVQYTIEVGSPLLTADTISALTEPGNRDAIAYKKRVYNDGNNAIEPEPRFAVKSETHRGIFRTAVGRRTGDLGKEDHEDCTCDMCIIFGNEHESSKIRFEDLELINGNEFEKLEKHIDHVAIDRFTGGALDKAKFDTYPLAGSPKKPLKLKGRFWIKKGFSGDHKLLITTALSDIRDGLYPLGSKGGVGYGWVAGISIDDNVPDDFKEMINKTEMPLPEEVEESNNGPINNDYVHPGHQSPKQDHKNKNIYYPHYFLDSGSKVYREKDIITHEEFTEELLSGKINCKLETLTPLIIPDTSDENGLKLQGNKPGHKNYKFFNINGELMIPGSELRGMLRTHFEALTKSCFAIFGEDSTLSWRMNADEKDYKIDSNSIRKMESQRNPKYRIPDELQKELRNSGNGLFNRLYTSERRFWSDVSNKFENSIDYKREILRCAGRPKNYKGGIIRQRKDSLMAEELKVHRLPLYDNFDIPDSAYKANDHCRKSATCSTSRGCRERFTCGIKVRDKNRVFLNAANNNRQYLNNIKKSNHDLYLQYLKGEKKIRFNSKVITGSERSPIDVIAELNERGRQTGFIKLSGLNNSNKSQGNTGTTFNSGWDRFELNILLDDLETRPSKSDYPRPRLLFTKDQYEYNITKRCERVFEIDKGNKTGYPVDDQIKKNYEDILDSYDGIKDQEVAERFDTFTRGSKLKVGDLVYFHIDGDNKIDSLIPVRISRKCASKTLGGKLDKALHPCTGLSDGLCPGCHLFGTTDYKGRVKFGFAKYENGPEWLITRGNNPERSLTLGVLESPRPAFSIPDDESEIPGRKFYLHHNGWRIIRQKQLEIRETVQPERNVTTEVMDKGNVFSFDVRFENLREWELGLLLQSLDPGKNIAHKLGKGKPYGFGSVKIKIDSLHTFKINSNNDKIKRVPQSDIREYINKGYQKLIEWSGNNSIQKGNVLPQWHVIPHIDKLYKLLWVPFLNDSKLEPDVRYPVLNEESKGYIEGSDYTYKKLGDKDNLPYKTRVKGLTTPWSPWNPFQVIAEHEEQEVNVTGSRPSVTDKIERRDGKMV

SEQ ID NO: 112 - protein, 1811 aa, synthetic construct
MHTILPIHLTFLEPYRLAEWHAKADRKKNKRYLRGMSFAQWHKDKDGIGKPYITGTLLRSAVLNAAEELISLNQGMWAKEPCCNGKFETEKDKPAVLRKRPTIQWKTGRPAICDPEKQEKKDACPLCMLLGRFDKAGKRHRDNKYDKHDYDIHFDNLNLITDKKFSHPDDIASERILNRVDYTTGKAHDYFKVWEVDDDQWWQFTGTITMHDDCSKAKGLLLASLCFVDKLCGALCRIEVTGNNSQDENKEYAHPDTGIITSLNLKYQNNSTIHQDAVPLSGSAHDNDEPPVHDNDSSLDNDTITLLSMKAKEIVGAFRESGKIEKARTLADVIRAMRLQKPDIWEKLPKGINDKHHLWDREVNGKKLRNILEELWRLMNKRNAWRTFCEVLGNELYRCYKEKTGGIVLRFRTLGETEYYPEPEKTEPCLISDNSIPITPLGGVKEWIIIGRLKAETPFYFGVQSSFDSTQDDLDLVPDIVNTDEKLEANEQTSFRILMDKKGRYRIPRSLIRGVLRRDLRTAFGGSGCIVELGRMIPCDCKVCAIMRKITVMDSRSENIELPDIRYRIRLNPYTATVDEGALFDMEIGPEGITFPFVFRYRGEDALPRELWSVIRYWMDGMAWLGGSGSTGKGRFALIDIKVFEWDLCNEEGLKAYICSRGLRGIEKEVLLENKTIAEITNLFKTEEVKFFESYSKHIKQLCHECIINQISFLWGLRSYYEYLGPLWTEVKYEIKIASPLLSSDTISALLNKDNIDCIAYEKRKWENGGIKFVPTIKGETIRGIVRMAVGKRSGDLGMDDHEDCSCTLCTIFGNEHEAGKLRFEDLEVVEEKLPSEQNSDSNKIPFGPVQDGDGNREKECVTAVKSYKKKLIDHVAIDRFHGGAEDKMKFNTLPLAGSFEKPIILKGRFWIKKDIVKDYKKKIEDAMVDIRDGLYPIGGKTGIGYGWVTDLTILNPQSGFQIPVKKDISPEGTYSTYPSHSTPSLNKGHIYYPHYFLAPANTVHREQEMIGHEQFHKEQKGELLVSGKIVCTLKTVTPLIIPDTENEDAFGLQNTYSGHKNYQFFHINDEIMVPGSEIRGMISSVYEAITNSCFRVYDETKYITRRLSPEKKDESNDKNKSQDDASQKIRKGLVKKTDEGFSIIEVERYSMKTKGGTKLVDKVYRLPLYDSEAVIASIQFEQYGEKNEKRNAKIRAAIKRNEVIAEVARKNLIFLRSLTPEELKKVLQGEILVKFSLKSGKNPNDYLAELHENGTERGLIKFTGLNMVNIKNVNEEDKDFNDTWDWEKLNIFHNAHEKRNSLKQGYPRPVLKFIKDRVEYTIPKRCERIFCIPVKNTIEYKVSSKVCKQYKDVLSDYEKNFGHINKIFTTKIQKRELTDGDLVYFIPNEGADKTVQAIMPVPLSRITDSRTLGERLPHKNLLPCVHEVNEGLLSGILDSLDKKLLSIHPEGLCPTCRLFGTTYYKGRVRFGFANLMNKPKWLTERENGCGGYVTLPLLERPRLTWSVPSDKCDVPGRKFYIHHNGWQEVLRNNDITPKTENNRTVEPLAADNRFTFDVYFENLREWELGLLCYCLELEPGMGHKLGMGKPMGFGSVKIAIERLQTFTVHQDGINWKPSENEIGVYVQKGREKLVEWFTPSAPHKNMEWNGVKHIKDLRSLLSIPGDKPTVKYPTLNKDAEGAISDYTYERLSDTKLLPHDKRVEYLRTPWSPWNAFVKEAEYSPSEKSDEKGRETIRTKPKSLPSVKSIGKVKWFDEGKGFGILIMDDGKEVSISKNSIRGNILLKKGQKVTFHIVQGLIPKAEDIEIAK

SEQ ID NO: 113 - protein, 1527 aa, synthetic construct
MKSIPITLTFLEPYRILPWAEKGKRDKKEYLRGANYVRLHKDKNGKFKPYITGTLIRSAVLSAIEMLLDITNGEWNGKECCLAKFHTEGEKPSFLRKKPIYIRAEKDEICTSRETACPLCLILGRFDKAEKKEKDKEKFDVHFSNLNLYSSKEFSTIEELAPKRALNRIEQYTGKAQDYFTVYEALNKEFWTFKGRIRIKEDIYDKVTDLLFSALRCVEKIAGALCRIEIDKEPSQQKGFVKRQLSKQAKEDIEKIFQVVKDAQKLRLLSDCFRELTRMANKDELALPLGPEDDGHYLWDKIKVEGKTLRIFLRNCFSQYKDNWLCFCDEASKKGYQKYREKRHKLTDRELPTATPKHFAEKKDPQISPIYIDKDDKVYEWIIVGRLIAQTPFHFGDEEKAEGAILLTPDNRFRLPRTALRGILRRDLKLAGASACEVEVGRSEPCPCDVCKIMRVTLLDTVSEDLRDFLPELRKRIRINPQSGTVAEGALFDTEVGPEGLSFPFVLRYKCEKLPDSLTTVLCWWQEGLAFLSGESATGKGRFRLEINGAFVWDLQKGLFNYIKNHGFRGEERLFLEGNEAELEKMGIQINTELLQPEMIKKEKNFTDFPYDLIKYQLNISSPLLLNDPIRAIALYEGEGKAPDAVFFKKYVFENGKIEEKPCFKAESIRGIFRTAVGRIKNVLTKNHEDCICVLCHLFGNVHETGRLKFEDLKIVSGQEEKFFDHVAIDRFLGGAKEKYKFDDKPIIGAPDTPIVLEGKIWVKKDINDEAKETLSQAFSDINTGIYYLGANGSIGYGWIEEVKALKAPSWLKIKEKPNFEKDTSLNISAIMNEFKKDIQTLNLDKTYLPYGFLKLLEKVKRTSSPITHERFYENHLTGFIECSLKVLSPLIIPDTETPEKEENGHKYYHFLKIDNKPIIPGAEIRGAVSSIYEALTNSCFRVFGEKKVLSWRMEGKDAKEFMPGRVSKKKGKLYMVKMQALRLPVYDNPALANEIRSGSIYEKYKNSKVEIIFFQTVEGIRKFLRGNFNNVEWKKVLVTGIDPLAILPSQKIPGNDKWVKNLQSKISPVRGYFKFTGPNKIETKRREEEKDEKLRTKANKVSCLQKDKWYEAMHNHVEYKQDYTPPNSPKTEPLERPRNIPCFVCSDKEKIYRMTKRCERVFVSLGENAPKYEIPISAIKRYEVILSAYRENWERNKTPELFRTRLPGDGRTLNEDDLVYFRADENEKVKDIIPVCISRIVDEVPLIKRLSQELWPCVLAECPLLGFECKKCELEGLPEKIWFRINKDGLCPACRLFGTQIYKSRVRFSFAYAKNWKFYDGYITLPRLESPRATWLILKEKDKHYIKYKVCGRKFYLHNSTYEDIINNSKKEKEKKTENNASFEVLKEGEFTFKVYFENLENWELGLLLLSLTGLGEAIKIGHAKPLGFGSVKIEAKKIYFREEAGKFHPCEKADEYLKKGLNKLTSWFGKNEINEHMRNLLLFMTYYQNLPKVKYPDFDGYAKWRCSYVEQDKVEYFQNRWIVAS

SEQ ID NO: 114 - protein, 4 aa, synthetic construct
  REGION >4: note = The entire sequence of amino acids 1-4 can be repeated one to twelve times
GGGG

SEQ ID NO: 115 - protein, 6 aa, synthetic construct
  REGION >6: note = The entire sequence of amino acids 1-6 can be repeated one to twelve times
GGGGGG

SEQ ID NO: 116 - protein, 6 aa, synthetic construct
  REGION >6: note = The entire sequence of amino acids 1-6 can be repeated one to twelve times
GGSGGS

SEQ ID NO: 117 - protein, 4 aa, synthetic construct
  REGION >4: note = The entire sequence of amino acids 1-4 can be repeated one to twelve times
GGGS

SEQ ID NO: 118 - protein, 5 aa, synthetic construct
  REGION >5: note = The entire sequence of amino acids 1-5 can be repeated one to twelve times
GGGGS

SEQ ID NO: 119 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccaagtatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 120 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaaaagacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 121 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggataaggacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 122 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcataaggggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 123 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggataagcattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 124 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccataagctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 125 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgaagctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 126 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcgaagggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 127 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatgaaggctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 128 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctaagcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 129 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaaaagtttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 130 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaagggaaggtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 131 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaagaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 132 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttgaagcggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 133 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaagaaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 134 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaagaaggatcctaatgggaatagcggttacgaagaactcaaagacggggaattcaagaaagaagatcggcagaagaagctgaccacgccgtggacaccgtgggcatag

SEQ ID NO: 135 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaaaagggggaattcaagaaagaagatcggcagaagaagctgaccacgccgtggacaccgtgggcatag

SEQ ID NO: 136 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccaggtatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 137 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaaaggacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 138 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatagggacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 139 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatagggggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 140 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggataggcattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 141 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccataggctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 142 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgaggctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 143 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcgaggggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 144 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatgagggctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 145 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctaggcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 146 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaaaggtttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 147 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaagggagggtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 148 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaggaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 149 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttgaggcggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 150 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaggaaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaagacggggaattcaagaaagaagatcggcagaagaagctgaccacgccgtggacaccgtgggcatag

SEQ ID NO: 151 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaagagggatcctaatgggaatagcggttacgaagaactcaaagacggggaattcaagaaagaagatcggcagaagaagctgaccacgccgtggacaccgtgggcatag

SEQ ID NO: 152 - DNA, 4806 bp, synthetic construct
  source 1..4806: mol_type = other DNA
atgacgactactatgaagatttcaattgaattcctcgagccgtttcggatgaccaaatggcaggaaagcactaggcgaaacaagaacaacaaggaatttgttcgcggccaagcttttgcaagatggcatagaaataagaaggataacacaaaagggcggccttacataactgggacattgctgcggtccgcagtaattagaagcgcggaaaacctgctgacattgagtgatgggaaaattagcgaaaagacatgttgtcctgggaaatttgatacggaagataaagacaggttgctgcaattgaggcagcggtccacccttcgttggaccgataagaacccctgtccagataacgctgaaacttactgcccgttttgcgaacttctcgggagatccggtaacgatggaaagaaggctgagaagaaggattggcgctttcgtattcattttggcaatctttcccttccagggaagcccgatttcgacgggccaaaagctataggcagccaacgggtacttaacagggtcgatttcaaatccggtaaggcccatgacttctttaaggcttacgaagtcgaccatactaggtttccccgcttcgaaggggagattaccatagataataaagtcagcgctgaagccaggaaactgctctgtgattctctcaagtttacggatcggctgtgtggagctctgtgcgtaatcaggttcgatgaatatacaccagcagccgatagtgggaaacaaaccgaaaatgtccaagcagaaccgaatgctaatctcgctgaaaagaccgcagagcaaattattagcattctggacgataataagaaaaccgaatatacccgcctgcttgctgatgctattcgttcactccgccgctctagtaaacttgtcgctggcttgccaaaggatcatgacgggaaggatgaccattacctctgggatatcggcaagaagaagaaagacgaaaactctgtcactattaggcaaatccttacgacctcagcagacaccaaggaactcaagaatgccggaaaatggagagaattctgcgagaagctgggtgaagcgctgtacctcaagagtaaagacatgagtggcggcctgaaaattactcggagaatactgggcgatgctgaattccatggaaagcccgatcggcttgaaaagagccggtctgtgtcaatcggatctgtgttgaaagaaactgtggtatgcggcgaactggtcgctaaaacgcccttcttcttcggagcgatagacgaagatgcaaaacaaaccgacctgcaagtactcctcactcccgataacaagtatagactgccaagaagcgccgtgcgaggtatactccgtcgagatcttcaaacctattttgatagcccatgcaatgctgagttgggtggacggccatgcatgtgtaagacgtgtaggattatgagagggatcacggtgatggatgcgcgcagtgagtacaatgccccgccagaaataaggcatcgtacccgcattaatcccttcacaggcacggttgcggaaggtgccctgtttaatatggaagtagcccccgaggggattgtctttccatttcaactccggtaccggggctctgaagatgggctgcccgatgcactgaaaacggtgttgaaatggtgggctgaggggcaggcattcatgagtggcgctgcctcaaccgggaagggccgattccggatggaaaatgctaaatatgaaacgctggatctgagcgacgagaatcaaaggaatgactatcttaagaattggggatggcgtgacgagaaggggctcgaggaactgaagaaacgactgaactcaggtctgccagagcccggtaattatagggatccaaaatggcacgagattaacgtttccattgagatggcaagcccttttattaatggcgacccaatccgcgcagccgtggacaaacgtggtacagatgtggttaccttcgttaagtataaagctgaaggggaagaggcgaaacccgtatgtgcatacaaggccgaatcttttagaggggtgatcagaagtgccgtggcacgcattcatatggaagatggcgtccctttgactgagttgactcacagtgactgtgaatgtctcctgtgccaaatctttggaagtgagtatgaagccggcaaaataaggtttgaagatctcgtattcgaaagtgacccggaacctgtgaccttcgatcatgtggccatcgatagattcactggcggtgcagctgataagaagaaattcgatgattcccctctgcccggtagccctgcaagaccgctcatgttgaaaggctccttctggatccgcagggacgttctcgaggacgaagagtactgtaaggcactcggtaaggctcttgcagatgtgaataatggcctttatcccctcggtggaaagagcgccatcggctacggacaggtcaagagtctgggtataaagggagatgataagaggatttctcgcctcatgaatcctgcctttgatgagacagatgtagccgttccagaaaagcccaaaactgatgccgaggttcgcatcgaggcagagaaagtatattacccacactatttcgtcgaaccccataagaaggtggaacgcgaggagaaaccctgtggtcatcaaaagttccacgaggggcgactgacaggtaaaattcggtgtaagctcattaccaagacacccctcatcgtcccagatactagtaatgacgatttcttcagacctgcggataaagaagctcggaaggaaaaggacgaatatcataaatcatatgctttcttcagacttcataaacaaatcatgattcccgggagcgaattgagaggaatggtgagtagtgtctacgaaactgtgacaaattcttgcttcaggatatttgatgagactaaacggttgtcatggcggatggatgctgatcaccagaatgtgctgcaagactttctcccaggtcgagtgaccgccgatgggaaacatatacaaaagttttccgaaactgcaagggtgcctttctatgacaaaacgcagaaacattttgacattcttgatgagcaagaaattgctggtgagaaacctgttcggatgtgggtcaagcgttttattaaacgactgagcctcgttgatcctgctaaacacccccagaagaaacaagacaataaatggaaaagacgcaaagaaggcatcgccacatttatagagcaaaagaatggctcttattattttaacgtggtcaccaataatggctgcacttctttccacttgtggcataaacctgacaattttgaccaggagaaactcgaaggcatacagaatggtgagaaactggattgctgggtaagagatagtagataccaaaaggcctttcaagagatacccgagaacgacccagacggatgggagtgtaaagagggctaccttcatgtcgtcggccccagcaaagtagagtttagtgacaagaaaggggatgtgatcaataactttcaaggaacactcccatcagttcccaacgactggaaaacaattaggacgaacgattttaagaataggaaaaggaagaatgaacctgtgttttgttgtgaggatgataagggcaattactataccatggctaaatattgcgaaaccttcttcttcgatctgaaggagaacgaggaatacgagatccccgagaaagcaagaatcaaatacaaagaactgctcagggtctataacaacaatcctcaagcagtgccggagagcgtatttcagtctagagttgcccgggaaaacgtggaaaagctgaagtccggagatcttgtgtatttcaaacataatgaaaagtacgtagaggacatcgtcccagtgcggatttcccgaactgtagacgataggatgatcggcaaacgtatgagcgccgatctgcggccgtgccatggagattgggtggaagatggtgatctcagtgccttgaatgcatatcccgagaaaagactcctcttgcgccaccccaaaggactctgccctgcttgccggctctttggaaccggatcttacaagggcagagtcaggtttggattcgcgtcactcgaaaacgatccggagtggctgatcccaggcaagaatcccggcgatccgtttcacggcgggccggtgatgctctcattgttggaacggcctcgcccgacttggagtataccgggatccgacaataagtttaaagtgcctggcagaaagttttacgtccaccaccacgcctggaaaaccattaaggacgggaaccatcccacaacaggcaaagctattgaacaaagccctaataaccgcactgtagaagctctcgccggcgggaattcctttagcttcgaaattgcctttgagaacctgaaagaatgggagctgggtttgctcatccacagcctgcaactcgaaaagggtctggcgcataaacttggaatggcaaagtctatgggatttggttcagttgaaattgacgtcgaatcagtgcgcctgagaaaagattggaagcaatggcggaatggcaattccgaaattcccaactggttgggaaaaggatttgctaaactgaaggaatggttccgggacgagctcgattttatagaaaatcttaagaaacttctttggtttcctgagggcgaccaagcaccccgggtttgctaccccatgctgcgaaagaaggacgatcctaatgggaatagcggttacgaagaactcaaaaggggggaattcaagaaagaagatcggcagaagaagctgaccacgccgtggacaccgtgggcatag